SEQUENCE LISTING (19 sequences)

SEQ ID NO: 1 - DNA, 90 bp, Artificial
  target dsDNA fragment
cgacgtcgacgtcggcgccgacgtcgccggcgacgtcgacgtcggcgccgacgtcgccggcgacgtcgacgtcggcgccgacgtcgccgg

SEQ ID NO: 2 - DNA, 90 bp, Artificial
  target dsDNA fragment
gctgcagctgcagccgcggctgcagcggccgctgcagctgcagccgcggctgcagcggccgctgcagctgcagccgcggctgcagcggcc

SEQ ID NO: 3 - DNA, 90 bp, Artificial; identical to SEQ ID NO: 1
  target dsDNA fragment

SEQ ID NO: 4 - DNA, 90 bp, Artificial; identical to SEQ ID NO: 2
  target dsDNA fragment

SEQ ID NO: 5 - DNA, 60 bp, Artificial
  target dsDNA fragment
cgacgtcgacgtcggcgccgacgtcgccggcgacgtcgacgtcggcgccgacgtcgccgg

SEQ ID NO: 6 - DNA, 60 bp, Artificial
  target dsDNA fragment
gctgcagctgcagccgcggctgcagcggccgctgcagctgcagccgcggctgcagcggcc

SEQ ID NO: 7 - DNA, 60 bp, Artificial; identical to SEQ ID NO: 5
  target dsDNA

SEQ ID NO: 8 - DNA, 60 bp, Artificial; identical to SEQ ID NO: 6
  target dsDNA sequence

SEQ ID NO: 9 - DNA, 30 bp, Artificial
  target dsDNA fragment
cgacgtcgacgtcggcgccgacgtcgccgg

SEQ ID NO: 10 - DNA, 30 bp, Artificial
  target dsDNA fragment
gctgcagctgcagccgcggctgcagcggcc

SEQ ID NO: 11 - DNA, 30 bp, Artificial; identical to SEQ ID NO: 9
  target dsDNA fragment

SEQ ID NO: 12 - DNA, 30 bp, Artificial; identical to SEQ ID NO: 10
  target dsDNA fragment

SEQ ID NO: 13 - DNA, 90 bp, Artificial
  target dsDNA fragment
cgacgtcgacgtcggcgccgacgtcgccggcgacgtcgacgtccgcgccgacgtcgccggcgacgtcgacgtcggcgccgacgtcgccgg

SEQ ID NO: 14 - DNA, 90 bp, Artificial
  target dsDNA fragment
gctgcagctgcagccgcggctgcagcggccgctgcagctgcaggcgcggctgcagcggccgctgcagctgcagccgcggctgcagcggcc

SEQ ID NO: 15 - DNA, 90 bp, Artificial; identical to SEQ ID NO: 13
  target dsDNA fragment

SEQ ID NO: 16 - DNA, 90 bp, Artificial; identical to SEQ ID NO: 14
  target dsDNA fragment

SEQ ID NO: 17 - DNA, 827 bp, Artificial
  synthetic construct
gaccaatccccagtgattatgcaagacagcggaccaatcagctccgccagctcatgaatatttatgaccttcgctgagtcaaagctttgaaccgagtttggggagctcagcagcatcatgcttagacttttcaaagagacaaactccattttcttatgaatggaaagtgaaaacccctgttccgcttaaattgggttccttcctgtcctgagaaacatagagacccccaaaagggaagcagaggagagaaagtcccacacccagaccccgcgagaagagatgaccatgaccaccatgccagaaagtctcaacagccccgtgtcgggcaaggcggtgtttatggagtttgggccgcccaaccagcaaatgtctccttctcccatgtcccacgggcactactccatgcactgtttacactcggcgggccattcgcagcccgacggcgcctacagctcagcctcgtccttctcccgaccgctgggctacccctacgtcaactcggtcagcagccacgcatccagcccctacatcagttcggtgcagtcctacccgggcagcgccagcctcgcccagagccgcctggaggacccaggtacgtgcgcttgccagggagagggagaggaggaggtacaagggagagagggaaagaaggagcgggggagaagaggagagggagagagagagaaagagaagagaggagagcgaggtggggtgggggtggggagggcgcgggagcagtggaggtttcgaatatcaatctatagatccttgtcacagcaaataaatttttttaaaaattccctcaatttgcaactatccagccaagg

SEQ ID NO: 18 - DNA, 27 bp, Artificial
  PCR primer
gaccaatccccagtgattatgcaagac

SEQ ID NO: 19 - DNA, 26 bp, Artificial
  PCR primer
ctcaatttgcaactatccagccaagg

(19) United States
(12) Patent Application Publication
(10) Pub. No.: US 2017/0022546 A1
(43) Pub. Date: Jan. 26, 2017

(54) DETECTION AND QUANTIFICATION OF METHYLATION IN DNA

(71) Applicants: Rashid BASHIR, Champaign, IL (US); Bala Murali VENKATESAN, San Diego, CA (US); George VASMATZIS, Oronoco, MN (US); Jiwook SHIM, Savoy, IL (US); The Board of Trustees of the University of IIIinois, Urbana, IL (US); Mayo Foundation for Medical Education and Research, Rochester, MN (US)

(72) Inventors: Rashid BASHIR, Champaign, IL (US); Bala Murali VENKATESAN, San Diego, CA (US); George VASMATZIS, Oronoco, MN (US); Jiwook SHIM, Savoy, IL (US)

(21) Appl. No.: 15/125,048

(22) PCT Filed: Mar. 10, 2015
(86) PCT No.: PCT/US15/19630

§ 371 Date: Sep. 9, 2016

Related U.S. Application Data
(60) Provisional application No. 61/950,828, filed on Mar. 10, 2014.

(51) Int. Cl. C12Q 1/68; G01N 33/487

(52) U.S. Cl. CPC C12Q 1/6825; G01N 33/48721

(57) ABSTRACT

Provided are methods and systems for characterizing a biomolecular parameter of a polynucleotide. A polynucleotide of interest from a sample comprising a heterogeneous mixture of polynucleotides is concentrated and provided to a first fluid compartment of a solid-state nanopore. An electric potential is established across the solid-state nanopore to force the polynucleotide of interest from a first fluid compartment to a second fluid compartment via the nanopore. A passage parameter output is monitored during passage of the polynucleotide of interest through the nanopore, wherein the passage parameter output depends on the biomolecular parameter status of the polynucleotide of interest. In this manner, the methods and systems are compatible with a wide range of applications, including epigenetic modifications to DNA indicative of a disease state such as cancer, in an integrated, reliable and low cost system.

### CROSS-REFERENCE TO RELATED APPLICATIONS

This application claims the benefit of priority from U.S. Provisional Patent Application No. 61/950,828 filed Mar. 10, 2014, which is hereby incorporated by reference in its entirety to the extent not inconsistent herewith.

### STATEMENT REGARDING FEDERALLY SPONSORED RESEARCH OR DEVELOPMENT

This invention was made with government support under R21 CA155863 and NCI R25 CA154015 awarded by the National Institutes of Health. The government has certain rights in the invention.

### BACKGROUND OF INVENTION

Provided are methods and related systems for characterization of polynucleotides that traverse a nanopore under an applied electric field. There is a need in the art for system that can: (1) detect target molecules at low concentrations from minute sample volumes; (2) detect biomolecular parameters of the target molecules, such as a combination of methylation aberrations across a variety of genes (important in monitoring disease progression and prognosis); (3) detect subtle variations in biomolecular parameters, such as methylation patterns across alleles, that would not be detected using bulk ensemble averaging methods such as PCR and gel-electrophoresis; (4) perform rapid analysis, such as methylation analysis; (5) reduce cost and simplify steps of experiment and analysis by eliminating cumbersome PCR, DNA sequencing and bisulfite conversion steps while maintaining sensitivity.

The methods and systems provided herein address these needs by providing a specially configured and integrated system that collects polynucleotides of interest from a sample and concentrates them in a desired region around a nanopore entrance. In this manner, highly sensitive and accurate characterization of polynucleotides is possible, with important applications in the field of medical testing, diagnostics and fundamental research.

### SUMMARY OF THE INVENTION

Methods and related systems are described for characterizing a polynucleotide property in a reliable, low cost and efficient system without sacrificing or impacting sensitivity and resolution. This is achieved, in part, by special handling and processing of biological samples that facilitate subsequent capture and concentration of a polynucleotide of interest in a well-defined region adjacent to a nanopore entrance. In this manner, the need for polynucleotide amplification or more specialized isolation and involved handling is avoided, while ensuring even a small level of a polynucleotide of interest in a sample is provided to and detected by the nanopore. This provides access to a platform that is well-integrated, with a range of applications related to characterization of polynucleotides, including in assays for various diseases and diagnostics related thereto.

In an aspect, the invention is a method for characterizing a biomolecular parameter of a polynucleotide by concentrating a polynucleotide of interest from a sample comprising a heterogeneous mixture of polynucleotides; providing the concentrated polynucleotide of interest to a first fluid compartment of a solid-state nanopore, wherein the solid-state nanopore separates the first fluid compartment from a second fluid compartment, and a nanopore fluidically connects the first fluid compartment and the second fluid compartment; establishing an electric potential across the solid-state nanopore to force the polynucleotide of interest from the first fluid compartment to the second fluid compartment via the nanopore; and monitoring a passage parameter output during passage of the polynucleotide of interest through the nanopore, wherein the passage parameter output depends on the biomolecular parameter status of the polynucleotide of interest.

The methods and systems are compatible with a wide range of biomolecular parameters, depending on the application of interest. Examples include, an oxidative modification; an epigenetic modification; and a nucleotide sequence of interest. For example, if known DNA sequences are associated with a disease state, such as a mutation that indicates a predisposition to cancer, or that is associated with cancer, the nucleotide sequence of interest may correspond to that sequence. Accordingly, any of the methods provided herein may use a probe or biomarker that is selective to that sequence.

The methods and systems provided herein are particularly suited for characterization of a biomolecular parameter that is methylation, such as hypermethylation or a pattern of methylation sites in the polynucleotide of interest.

The methods and systems provided herein may further comprise the step of introducing a biomarker to the polynucleotide of interest prior to passage of the polynucleotide of interest through the nanopore, wherein the biomarker specifically binds to a polynucleotide of interest having the biomolecular parameter. As described, such biomarkers are useful for increasing sensitivity with respect to determining presence or absence of a biomolecular parameter for a polynucleotide transiting the nanopore. For example, it can be difficult to reliably resolve methylated from unmethylated DNA solely by nanopore transit. This difficulty is addressed herein by use of a biomarker, so that methylated versus not methylated DNA is identified. In a similar manner, any number of biomolecular parameters can be characterized for biomarkers that have specific binding to the biomolecular parameter status. Use of such a biomarker is indicated for those polynucleotide sequences and nanopore geometry wherein the presence or absence of the biomolecular parameter does not result in a reliable difference in passage parameter output. In contrast, a biomarker that is specific to the biomolecular parameter condition can effect a relatively large change in a passage parameter output, thereby allowing a user to distinguish between a polynucleotide of interest without the biomolecular parameter (e.g., no biomarker bound) from an equivalent polynucleotide of interest with the biomolecular parameter (e.g., with a biomarker bound).

In an embodiment, the biomarker is selected from the group consisting of: a methylation binding protein; a sequence-specific binding motif; an antibody specific to a nucleotide-binding protein; a base excision repair protein; and a nucleotide-binding protein. Exemplary specific biomarkers may include at least one of: Uhrf, MBD, Kaiso family, ZBTB4 or ZBTB38, and the biomolecular parameter is methylation of DNA. As discussed, the invention is compatible with any biomarker that is associated with a biomolecular parameter, and that provides a substantial change in a process parameter output for the nanopore.

As desired, any number of passage parameter outputs may be monitored, measured or calculated. Examples include blockade current, nanopore transit time, or both blockade current or nanopore transit time.

In an aspect, the blockade current for a methylated DNA polynucleotide:MBD complex is at least 2-fold greater than a blockade current for a corresponding unmethylated DNA polynucleotide traversing the nanopore. Similarly, the method may further relate to a biomarker:polynucleotide of interest complex that provide an at least 2-fold difference, at least 3-fold, or at least 5-fold difference in the passage parameter output compared to polynucleotide of interest transiting without a biomarker.

In an aspect, there is a biomarker to polynucleotide of interest ratio that is greater than or equal 1:1, 1.5:1, or 3:1, or selected from a range that is between 1:1 and 5:1.

The methods and systems provided herein are compatible with a range of polynucleotides, such as single stranded DNA, double stranded DNA or RNA. A particular advantage of the systems and methods herein is the compatibility with a range of polynucleotide lengths, ranging from short, less than 100 base pairs, to long, such as greater than 800 base pairs, and intermediate lengths thereof. In an aspect, the polynucleotide has a nucleotide length that is greater than or equal to 30 nucleotides and less than or equal to 100 nucleotides. Accordingly, any of the samples used in the assay, may be processed to provide smaller polynucleotide lengths, such as by restriction enzymes, thermal digestion, and the like, without having to amplify the polynucleotides.

Examples of passage parameter output include any of: blockade current, threshold voltage, pattern of blockade current, frequency of blockade current, duration of blockade current, translocation velocity, translocation time, and statistical parameters thereof, such as averages. In an aspect, a biomarker is provided to bind to the polynucleotide of interest, wherein a binding complex comprising the biomarker and polynucleotide of interest changes an average passage parameter output value by at least 100% compared to a polynucleotide of interest without the bound biomarker. For example, there may be an at least 2-fold increase in blockade current or transit time, including between 2-fold and 10-fold. “Threshold voltage” is used herein to indicate a driving voltage required to force a polynucleotide through the nanopore, so that without a biomarker, the threshold voltage may be much smaller than compared to a polynucleotide having the biomarker connected thereto.

Depending on the application of interest, the nanopore has an average diameter that is greater than or equal to 5 nm and less than or equal to 12 nm. In an aspect, the solid state nanopore comprises a dielectric membrane having a thickness less than or equal to 20 nm. The dielectric membrane may comprise SiN, Al₂O₃, graphene, or HfO₂, and multi-stacked layers thereof. In an aspect, the dielectric membrane comprises graphene having a thickness of less than 0.5 nm through which the nanopore traverses.

The systems and methods are compatible with a range of samples, with the sample selected depending on the application of interest. For example, the sample may comprise a biologic sample obtained from an individual, the biological sample selected from the group consisting of a blood sample, a stool sample, urine sample, saliva or sputum sample, or a tissue sample.

A unique aspect of the systems and methods provided herein is the integrated aspect wherein polynucleotides of interest in a sample are specifically concentrated at or near a nanopore entrance. In an aspect, the concentrating step comprises: binding the polynucleotide of interest to a capture element; separating unbound polynucleotides from the bound polynucleotides of interest; and releasing the polynucleotide of interest from the capture element. The released polynucleotide of interest may be transported to the first fluid compartment.

Alternatively, the capture element may be positioned in the first fluid compartment and polynucleotides of interest provided to the positioned capture element. In this manner, the capture element ensures that polynucleotide of interest is concentration in the first fluid compartment.

Any of the methods may further comprise the step of introducing a biomarker specific to the polynucleotide of interest before or after binding of the polynucleotide of interest to the capture element. A biomarker specific to the polynucleotide of interest may be introduced: after binding of the polynucleotide of interest to the capture element; or after releasing of the polynucleotide of interest from the capture element. Alternatively, the biomarker may be part of the capture element, such as connected to the capture element and used to specifically capture a polynucleotide of interest having the biomolecular parameter. For example, a methyl binding domain may be connected to a surface of a capture element comprising a bead, so that the specific binding property of the methyl binding domain facilitates specific binding of polynucleotide of interest having methylated nucleotides (e.g., methylated cytosine) to the bead. Similar targeting of biomarkers that specifically bind to other biomolecular parameters are compatible with the instant methods and systems. In this manner, the capture element is specific to the polynucleotide of interest having the biomolecular parameter present.

The functional benefit of the systems and methods may be characterized in terms of an increase in concentration of the polynucleotide of interest, particularly at or near the nanopore entrance. In an aspect, the concentrating step increases a polynucleotide of interest concentration by at least a factor of 500 in a region adjacent to the nanopore compared to the polynucleotide of interest concentration in a region that is not adjacent to the nanopore, such as a range of between 500 and 10,000 or between 500 and 2,000, and any sub-ranges thereof. A region may be considered “adjacent” to a nanopore if, upon energization of the driving electric field, the polynucleotide is forced into contact with the nanopore entrance. If after a user-selected time, the polynucleotide has not entered the nanopore, that location where the polynucleotide initially started may be considered to be not adjacent to the nanopore entrance. Of course, this functional definition will depend on operating conditions, such as strength of electric field, electrolyte composition, nanopore size, polynucleotide size. Accordingly, adjacent may also be defined in terms of absolute values, such as within 500 μm, 250 μm or 100 μm of a nanopore. Adjacent may also be defined in terms of a chamber that is provided around the nanopore passage, with an according volume. Capture elements, or components thereof, may be matched with the region considered to be adjacent, such that polynucleotides of interest are forced into the region, such as by application of a magnetic force, electric field, bulk fluidic convection and the like.

In an aspect, the first fluid compartment has a sample-containing volume that is fluidically adjacent to a nanopore entrance and that is less than or equal to 500 μL, 250 μL, or 100 μL. In this manner, the polynucleotide concentration is increased dramatically, simply by virtue of ensuring the polynucleotide is forced into this region. Accordingly, polynucleotide amplification is avoided.

In an aspect, the transport of the polynucleotide of interest to the first fluid compartment is by a microfluidic channel. For example, the microfluidic channel may directly convey the sample, or the sample may have undergone upstream processing so that the polynucleotide has been pre-processed. For example, the polynucleotide may be bound to a particle or bead having properties conducive to subsequent capture. The microfluidic channel may have a characteristic dimension that is less than 1 mm, less than 100 μm, or between about 1 μm and 100 μm, or between 1 μm and 20 μm.

In an embodiment, the capture element comprises a magnetic bead to which the polynucleotide of interest is attached, and the capture element is suspended in a microfluidic channel. Alternatively, the bead may have other properties conducive for capture by other forces, such as an electrostatic force, such as by electrophoresis.

In an aspect, the concentrating step further comprises: applying a magnetic force to drive the magnetic bead with polynucleotide of interest from the microfluidic channel to a first fluid compartment region fluidically adjacent to a nanopore entrance; introducing a cleavage element into the microfluidic channel and fluidically flowing the cleavage element to the first fluid compartment region to cleave the polynucleotide of interest from the magnetic bead at a cleavable linker site; wherein the establishing the electric potential step forces polynucleotide of interest in the first fluid compartment region to the nanopore entrance and through the nanopore and the monitoring the passage parameter output distinguishes between biomarker and polynucleotide of interest complexes traversing the nanopore from polynucleotide of interest without biomarker traversing the nanopore.

The cleavage element during the establishing the electric potential step may be positively charged, and the established electric field forces the cleavage element in a direction that is away from the nanopore entrance. This advantageously minimizes risk of cleavage elements interfering with subsequent polynucleotide nanopore transit and measurements related thereto.

The cleavable linker site may be any number of elements given the range of specific cleavage mechanisms, including by restriction enzymes and the like that target specific sequences. One example of a suitable linker site comprises four uracils positioned between an amino conjugation terminal and a complementary sequence. In this manner, the paired cleavage element to that linker site may be a glycosylase that selectively cleaves the cleavable linker site.

The method may further comprise the step of introducing a biomarker into the microfluidic channel and fluidically flowing the biomarker to the first fluid compartment region to bind the biomarker to polynucleotide of interest having a biomolecular parameter that provides specific binding to the biomarker. In an aspect, the biomarker is a MBD protein.

The concentrating step may comprise providing the polynucleotide of interest to a first fluid compartment region having a confined volume. For example, the confined volume may be within 500 μm of an entrance of the nanopore, or have a confined volume that is less than or equal to 50,000 μm³. Alternatively, the confined volume may be defined in terms of a fraction of the first fluid compartment volume, such as a central portion that surrounds the nanopore entrance, such as 50% or less, 30% or less, or 10% or less of the first fluid compartment region. Alternatively, the first fluid compartment may be configured to have walls, or wall portions, that define edges of the confined volume.

The method of the present invention may comprise the step of directing a magnetic force through a microfluidic channel containing the polynucleotide of interest bound to a magnetic bead flowing through the microfluidic channel to capture magnetic beads within the confined volume. The magnetic force may be generated by a permanent magnet or a pattern of microfabricated magnets. The pattern of microfabricated magnets may comprise a ferromagnetic material arranged in a pattern to decrease velocity of the magnetic bead flowing in the microfluidic channel and to increase distribution uniformity of the magnetic beads in a region adjacent to the nanopore entrance. One example of a ferromagnetic material is nickel. For example, particles in a center streamline position in the microfluidic channel may be pulled toward a surface into a slower streamline position, particularly for laminar flow, thereby further increasing the likelihood of capture.

Any of the methods provided herein may further comprise the step of directing a magnetic force through a microfluidic channel containing a magnetic bead flowing through the microfluidic channel to capture magnetic beads within the confined volume, wherein the magnetic beads are coated with an oligonucleotide complementary to a target sequence of the polynucleotide of interest. A polynucleotide of interest may be provided to the magnetic bead to bind the polynucleotide of interest to the magnetic bead.

Other examples of capture elements include those based on electrokinetic techniques such as dielectrophoresis or isotachophoresis to concentrate the bead/DNA/protein complex around the nanopore. In an aspect, the capture element comprises a particle positioned within a concentrating electric field that directs the particle to the first fluid compartment. The particle may be a charged bead to which the polynucleotide of interest in attached. The concentrating electric field may be applied in a dielectrophoretic or isotachophoretic manner.

The invention may be further described in terms of selecting a nanopore passage geometry to provide an intermittent interaction between the polynucleotide of interest transiting the nanopore and an inner surface of the nanopore, corresponding to the biomolecular parameter, wherein the intermittent interaction is detectable as a change in passage parameter output. For example, the biomolecular parameter may comprise a nucleotide binding protein that is specific to the biomolecular parameter, including a biomolecular parameter of methylation and the nucleotide binding protein that is a MBD protein.

Any of the nanopores provided herein may be functionalized with an antibody for specific binding to the biomolecular parameter during transit of the polynucleotide of interest.

The methods and systems provided herein are particularly well suited for distinguishing those polynucleotides of interest not having the biomolecular parameter from those that do. For example, the polynucleotide of interest may comprise a plurality of polynucleotides formed from a first population of polynucleotides having the biomolecular parameter of interest and a second population of polynucleotides without the biomolecular parameter of interest. In this manner, the method may further comprise identifying a fraction of polynucleotides having the biomolecular parameter of interest. As desired, a plurality of systems may be employed to provide, for example, high-throughput screeing, such as by a plurality of nanopores.

The polynucleotide of interest may be present in the sample at a ratio of less than 1 polynucleotide of interest to 1000 total polynucleotides. The methods provided are capable of characterizing the biomolecular parameter at a polynucleotide of interest concentration that is as low as 1000 molecules/μL or about 1 fM. The method may screen a blood sample or a stool sample for a biomolecular parameter indicative of a disease state. Examples of disease states include cancer, neurodegeneration, single nucleotide polymorphisms associated with a genetic disease. The concentrating may, in turn, effectively increase the concentration at a region adjacent to the nanopore, such as about 500-fold or more than the original sample, 500,000 molecules/μl or about 500 fM.

In another embodiment, the concentrating step may comprise providing a bead having a probe connected to a surface of the bead that specifically binds to a polynucleotide of interest. For example, the probe may comprise a biomarker that specifically binds to a polynucleotide of interest having the biomolecular parameter to be characterized. The probe may comprise a methyl-binding protein that specifically binds a methylated region of the polynucleotide of interest. The methyl binding protein may bind to a hemi-methylated region of double-stranded DNA.

The methods and systems provided herein may have a sensitivity capable of detecting a single biomolecular parameter in the polynucleotide of interest, such as a single cytosine methylation in a polynucleotide of interest.

In another embodiment, the invention is a device, system, or assay for performing any of the methods provided herein. In an aspect, provided herein is an integrated diagnostic system comprising: a solid state nanopore that traverses a dielectric membrane, the nanopore having a diameter less than 20 nm, such as between about 5 nm and 18 nm; the membrane having a thickness less than 30 nm, such as between 1 nm and 30 nm, and a top and a bottom surface with the thickness extending therebetween. A nanopore entrance is coincident with the dielectric membrane top surface and a first fluid compartment is positioned adjacent to the dielectric membrane top surface. A first fluid compartment region is positioned within the first fluid compartment and fluidically adjacent to the nanopore entrance. The first fluid compartment may also correspond the first fluid compartment region. A nanopore exit is coincident with the dielectric membrane bottom surface, wherein the nanopore fluidically connects the first fluid compartment and the second fluid compartment. A power supply is electrically connected to the first fluid compartment and the second fluid compartment to provide an electric potential difference between the first fluid compartment and the second fluid compartment. This potential difference is used to force polynucleotides in the first fluid compartment region from the first fluid compartment to the second fluid compartment, via the nanopore. A detector is operably connected to the nanopore, the detector configured to monitor a passage parameter output for a polynucleotide traversing the nanopore under the electric potential difference between the first fluid compartment and the second fluid compartment. For example, any of current, resistance, capacitance or other electrical parameter through the nanopore may be detected. Similarly, other variables may be calculated therefrom, including transit time, transit velocity, and the like. A microfluidic passage is configured to fluidically transport a sample to the first fluid compartment region. A capture element positioned in the microfluidic passage and/or the first fluid compartment region captures and concentrates a polynucleotide of interest in the first fluid compartment region. A release element is in fluidic contact with the microfluidic passage for controllably releasing the polynucleotide of interest from the capture element to the first fluid compartment region. Upon energization of the power supply, the released polynucleotide of interest in the first fluid compartment region traverses the nanopore to the second fluid compartment.

The system may further comprise a biomarker in fluidic contact with the microfluidic passage for binding to a polynucleotide of interest having a biomolecular parameter that provides specific binding with the biomarker.

The system may further comprise a magnet positioned to provide a magnetic force to capture a capture element that is a magnetic particle at the first fluidic compartment region, wherein the first fluidic compartment region is within 500 μm of the nanopore entrance. The magnet may comprise a plurality of ferromagnetic elements arranged in magnetic contact with the microfluidic channel and in a pattern configured to decrease velocity of a magnetic particle flowing in the microfluidic channel, capture and uniformly distribute magnetic particles relative to the nanopore entrance. The capture pattern may be symmetrically aligned relative to the nanopore entrance perimeter, so that the captured particles are uniformly distributed out to a maximum separation distance from the nanopore, such as out to 500 μm, 250 μm, or 100 μm. “Uniformly distributed” is used herein to refer to a less than 30%, less than 20% or less than about 10% maximum deviation from average over the entire region.

In an aspect, at least 70% of all magnetic particles flowing in the microfluidic channel are captured by the magnetic force and positioned around the nanopore entrance.

The release element may comprise an enzyme that selectively cleaves the polynucleotide of interest from the magnetic particle at a cleavable linker site to release polynucleotide of interest to the first fluidic compartment region.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIGS. 1A-1F illustrate detection of methylated and unmethylated DNA using a solid-state nanopore. FIG. 1A Schematic diagram of a nanopore setup. A focused electron beam of TEM sculpts a nanopore in a thin (˜20 nm) silicon nitride membrane; the nanopore chip is sealed between two fluidic cell chambers containing conductive electrolyte; a voltage is applied, such as by a power supply, across this setup to induce the translocation of single dsDNA molecules through the pore as shown. The inset is a TEM image of typical ˜4.2 nm diameter nanopore used in DNA measurements (scale bar is 10 nm). FIG. 1B Characteristic ionic current traces measured during translocation of mDLX1 (827 bp dsDNA with 36 potential CpG methylation sites). Traces are recorded in 600 mM KCl at pH 8.0 electrolyte at various voltage levels. FIG. 1C A typical DNA induced current blockade. Parameters of interest are the open pore current, I_{O}, residual blocking current, I_{B}, (occurs while a single DNA molecule translocates through the nanopore), blockage current ΔI=I_{B}−I_{O}, and translocation time of the molecule, t_{duration}. FIG. 1D Schematic showing (top) the chemical difference between cytosine and one form of methylated cytosine; (middle) unmethylated versus a fully methylated CpG dinucleotide in dsDNA. Data traces of unmethylated- (bottom-left) and methylated-DLX1 (bottom-right) recorded at 300 mV driving potential, showing similarity between both data traces. FIG. 1E Comparison of mDLX1 and uDLX1 transport. ΔI and T_{d} plots as a function of applied voltage. T_{d} and ΔI refers to the time constant and the blocking current respectively at each voltage. All points are the value of the fit with standard error. Second order of polynomial fit to/and exponential fit to T_{d} are also shown in short dash (black fits to uDLX1 and red to mDLX1). Each data are overlaid with over n=1167 separate translocation events recorded per data point. Methylated and unmethylated fragments are not readily indistinguishable from each other. FIG. 1F T_{d} (top) and A/(bottom) histograms for mDLX1 versus uDLX1 at 50 mV (n>2153), showing similarity with T_{d}_{_}_{mDLX1}=0.124±0.006 ms, T_{d}_{_}_{uDLX1}=0.135±0.006 ms, ΔI_{—mDLX1}=−449.5±5.4 pA and ΔI_{—uDLX1}=−440.8±24.3 pA.

FIGS. 2A-2F illustrate differentiation of unmethylated DNA from mDLX1/MBD-1x complex. FIG. 2A Structure of B-form dsDNA (left) and methylated DNA/MBD complex (right). A single MBD protein binds to the methylated CpG site on the major groove of dsDNA, occupying about 6 bps (PDB ID: 1IG4). FIG. 2B Top-down view: the cross-sectional diameter of the complex with a single bound MBD protein is −5 nm. Multiple bound proteins along the DNA major groove increase complex diameter to ˜7.6 nm. FIG. 2C Gel-shift assay showing the high affinity and specificity of MBD-1x for methylated but not unmethylated DNA. When increasing amounts of MBD-1x protein are incubated with uDLX1, no DNA-protein complex is formed (lanes 1-3), but when mDLX1 is included a robust, dose-dependent increase in mDLX1-MBD-1x complex formation is observed (lanes 5-9) Lane 5 and 9 show 1:5 and 1:30 (mDLX:MBD-1x), respectively. Samples are fractionated on an 8% non-denaturing polyacrylamide gel and visualized using autoradiography. FIG. 2D Nanopore ionic current traces recorded in 600 mM KCl, pH 8.0 at 600 mV; uDLX1 events (left), mDLX1/MBD-1x events (right) illustrating a robust difference in nanopore parameter this is blockade current. FIG. 2E Characteristic translocation signatures for uDLX1 (bottom) versus the complex (top) through a ˜12 nm pore. Scale bar is 10 nm in the TEM image. Qualitatively, the mDLX1/MBD-1x complex induces longer, deeper current blockades relative to uDLX1, indicating a passage parameter of transit time may also be used to distinguish methylated DNA:binding protein complex from unmethylated DNA without binding protein. FIG. 2F t_{duration} (left) and ΔI (right) histograms at 600 mV for uDLX1 (shown in blue—n=857) and mDLX1 (shown in red—n=197). Unmethylated DNA and the complex are clearly distinguishable. Exponential fits give time constants of T_{d}_{_}_{uDLX1}=0.103±0.005 ms and T_{d}_{_}_{mDLX1}=1.43±0.03 ms respectively.

FIGS. 3A-3E illustrate methylation quantification based on number of bound MBD-1x proteins. MBD-1x protein is incubated with methylated DLX1 DNA at ratios of FIG. 3A 1:30, FIG. 3B 1:5 and FIG. 3C 1:1. Characteristic current signatures representing the mDLX1/MBD-1x complex (top) and unmethylated DLX1 DNA (bottom) through 9-10 nm diameter pores are shown. Current signature histogram of unmethylated DLX1 (uDLX1—black) and methylated DLX1-MBD-1x complex (MCLX1/MBD-1X—red). Histogram is generated with peak current signature value of each event. The inset is a TEM image of a nanopore, with a scale bar of 10 nm. FIG. 3D Translocation time histograms representing the mDLX1/MBD-1x complex and unmethylated DLX1 (inset). FIG. 3E Methylation Detection (left): Complexes formed with any ratio of MBD-1x can discriminate from uDLX1 using blockage current alone (˜3-fold increase in blockage current induced by the complex is seen). Methylation Quantification (right): Complexes formed with different ratios of protein can be differentiated based on the number of bound MBD-1x molecules. Time constants for the complexes are shown by the red open circles: J_{1:30}=4.51±0.48 ms, J_{1:5}=1.67±0.17 ms and J_{1:1}=1.01±0.09 ms. Corresponding time constants for uDLX1 are in the range of 0.107-0.184 ms. MBD-1x on complexes are quantified with extended translocation duration. 1:1 complex shows ˜7-fold prolonged translocation duration, 1:5 at ˜12-fold and 1:30 at ˜31-fold respectively than unmethylated DNA.

FIGS. 4A-4D are Molecular Dynamics (MD) simulations of methylated DNA/MBD complex through a nanopore. Temporal MD snapshots showing translocation of 63 bp dsDNA with: FIG. 4A 3 bound MBD proteins through a 12 nm pore, FIG. 4B 3 bound MBD proteins through a 10 nm pore, FIG. 4C 1 bound MBD protein through a 9 nm pore. As pore size is reduced, hydrophobic interactions between the complex and the pore begin to dominate and can arrest the transport of the molecule through the pore. FIG. 4D Center of mass of the complex is shown distance vs. time. Smaller pore sizes can result in the trapping of the complex in the pore.

FIG. 5. DNA sequence (SEQ ID NO:17). The 827 bp DNA fragment includes a region of the DLX1 gene from the untranscribed area just downstream of a CpG island, through the 5prime UTR, the first exon and part of the first intron. It contains 36 methylated sites, including 4 Hhal sites, which are underlined. Matching bases in coding regions of cDNA are colored blue and capitalized. Matching bases in UTR regions of cDNA are colored red and capitalized. PCR primers are in pink.

FIG. 6. DLX1 promoter methylation in lung adenocarcinoma. Data are from gene expression Omnibus¹ which includes 58 lung adenocarcinoma (AD) and adjacent non-neoplastic lung (N) samples. Data points are signal intensities by cg15236866 probe on the IIlumina HumanMethylation 27 BeadChip. Hypermethylation of DLX1 promoter in AD compared with N is statistically significant (p<10⁻¹⁰).

FIG. 7. Methylation by CpG methyltransferase M.Sssl. Three methylated samples and one unmethylated sample. Odd numbered lanes contain 5 uL of Hhal digested samples. Even numbered lanes contain undigested sample in equal amounts. Lane 7 is unmethylated and digested. Compared to digested methylated samples in lanes 1, 3 and 5 there is no band of equal size as undigested, indicating complete digestion of unmethylated DNA. All other digests have bands, indicating all the four Hhal sites are methylated in those molecules. This implies that all of the molecules should be at least partially methylated.

FIGS. 8A-8C. Induction and purification of MBD-1x. FIG. 8A. E. coli BL21 (DE3) pLysS cells are treated (+) or not treated (−) with IPTG, lysates are subjected to SDS-PAGE, and stained with Coomassie blue. FIG. 8B. MBD-1x protein is refolded and eluted with increasing concentrations of imidazole (E1-E5). The eluted samples are subject to SDS-PAGE and stained with Coomassie blue. FIG. 8C. Purified MBD-1x is subjected to Western blot analysis using an anti-His antibody to detect the his-tagged MBD-1x.

FIG. 9. Schematic structure of multiple MBD-1x binding to 827 bp dsDNA with 36 methyl-CpG. The 827 bp dsDNA with 36 methyl-CpG can have MBD-1x binding to it from all around. Since the dsDNA turns at every 10.5 bp, the distance between 1^{st} CpG and a specific CpG is counted as number of base-pairs. Then we multiply (360/10.5) to the number of base-pairs, and divided by 360 degree and mark the angle, as indicated. The width of the molecule with multiple MBD-1x bound to the DNA is about 7.6 nm.

FIG. 10. Effect of increasing KCl concentrations on mDLX1/MBD-1x complex formation. Methylated DNA is incubated alone (lane1) or combined with 0.2 (lanes 2-5) or 0.8 (lanes 6-9) ng MBD-1x. 80 (lane 1, 2 and 6), 150 (lanes 3 and 7), 300 (lanes 4 and 8) and 600 (lanes 5 and 9) mM KCl is included in the binding buffer. Samples are fractionated on a 6% nondentaturing polyacrylamide gel and visualized using autoradiography.

FIGS. 11A-11E. Mixture of uDLX1 and mDLX1/MBD-1x through 4.5 nm pore. FIG. 11A. Nanopore TEM image, with scale bar indicating 10 nm). FIG. 11B. Data trace of mixture of 1 nM uDLX1 and 10 pM of mDLX1/MBD-1x complex. Most events are associated with uDLX1 translocation through the nanopore with occasional nanopore deep current blockade for an extended time period. The deeper blocking may be attributed to mDLX1/MBD-1x sitting at the entrance of the nanopore but not translocating through the nanopore due to the larger physical size than mDLX1/MBD-1x complex FIG. 11C. Scatter plot of events. FIGS. 11B-11C. No distinguishable events are detected. FIGS. 110-11E. Data traces of mixture. FIG. 11D. Data trace in 20 s. FIG. 11E. Detail-view of single events marked in FIG. 11D. Detail-view of individual single events supports the interpretation of that most events are associated with uDLX1 (mark 2, 3(left) and 5) and mDLX1/MBD-1x is bouncing at the entrance of the nanopore (mark 1, 3(right) and 4).

FIGS. 12A-12G. Mixture of uDLX1 and mDLX1/MBD-1x through 7 nm pore. FIG. 12A. Data trace of uDLX1 only (left), and mixture of uDLX1 and mDLX1/MBD-1x complex (right). Distinguishable deeper blocking current events are observed at data trace of mixture of uDLX1 and complex, while indistinguishable events are observed at data trace of uDLX1 only. Thus, the deeper blocking currents can be interpreted as mDLX1/MBD-1x complex events. FIG. 12B. Scatter plot of uDLX1 only events. FIG. 12C. Scatter plot of mixture of uDLX1 and mDLX1/MBD-1 complex. A few of deeper blocking currents were observed. Most of these events are in comparable translocation duration of uDLX1 (˜0.15 ms) and some showed prolonged translocation duration (>2 ms). FIG. 12D. TEM image of nanopore and the scale bar is 10 nm. FIGS. 12E-12G. Representative individual events extracted from data trace of mixture. FIG. 12E. Translocation of uDLX1, all is spike-like events at ˜1 nA blocking currents in ˜0.15 ms translocation duration. FIG. 12F. mDLX1/MBD-1x complex is bouncing at the nanopore entrance. Due to the very tight-fitting size between complex and the nanopore, the complex bounces at the nanopore entrance, but does not pass through the nanopore. Full current recovery in the middle of events is the evidence of complex bouncing at the nanopore entrance. When the molecule goes away from the nanopore entrance after bouncing, the nanopore has full opening thus it has full open pore current. Then complex comes back to the nanopore entrance by applied voltage. FIG. 12G. Some translocation of mDLX1/MBD-1x through the nanopore. Complex occasionally translocates through the nanopore and events are at very deeper blocking current (>4times of uDLX1). However, the pore clogged with the complex after a few deeper blocking current events, and did not recover.

FIGS. 13A-13F. Model of translocation of the different regions of the 827 bp methylated-DNA (with or without the MDB-1x) showing multiple or no CpG methyl-binding sites. FIGS. 13A-13C. Three regions on 827 bp methylated-DNA which have no CpG methyl-binding sites over 58 bps. FIG. 13A. dsDNA region between location 172658513 and 172658612. FIG. 13B. dsDNA region between location 172658603 and 172658702. FIG. 13C. dsDNA region between location 172659013 and 172659072. FIGS. 130-13F. Representative dsDNA regions with multiple CpG methyl-binding sites on 827 bp methylated-DNA. FIG. 13D. dsDNA region between location 172658703 and 172658792. FIG. 13E. dsDNA region between location 172658793 and 172658892. FIG. 13F. dsDNA region between 172658893 and 172659002. Refer to FIG. 5 for base location numbers.

FIG. 14. Control experiment for MBD-1x only in the solution. 300 pM of free MBD-1x is introduced in the pore in 600 mM KCl at pH 8.0. The proteins unbound with methylated DNA are not attracted into the pore by applied positive voltage across the nanopore, because MBD-1x is positively charged at pH 8.0. Sequence-specific isoelectric point of MBD-1x is 8.85 and is calculated according to the described sequence information.²

FIGS. 15A-15B. Discrimination of mDNA/MBD-1x from uDNA. FIG. 15A. Mixture of 1 nM of uDNA and 10 pM of mDNA/MBD-1x complex is introduced to the nanopore, and the complex is discriminated from uDNA by an about 3 times deeper current blockade and over 30 times prolonged translocation event. See FIG. 17 for detailed nanopore ionic current of mDNA/MBD-1x translocation event. FIG. 15B. Scatter plot of data trace and the pore image (scale bar in TEM image is 10 nm).

FIG. 16. Comparison of all-points blocking current histogram to mDLX1/MBD-1x complex in various ratios. All-point blocking current histogram of each mDLX1/MBD-1x complex ratio is superimposed with uDLX1 histogram. uDLX1 is in blue (toward right of the histogram) and mDLX1/MBD-1x is in red color (toward left of the histogram). Superimposed between uDLX1 and mDLX1/MBD-1x on current zero is open pore current, i.e. nanopore is not occupied. uDLX1 produces blocking current signature below ˜1 nA through all three nanopores, while complexes blocked nanopore with a current larger than ˜2 nA. Complexes in ratio of 1:30 and 1:5 show very little overlapping region between uDLX1 and mDLX1/MBD-1x complex, but complex of 1:1 ratio shows large overlapping region between all-points histogram peaks of uDLX1 and mDLX1/MBD-1x complex. This indicates that 1:1 ratio complexes translocate through the nanopore with blocking current signature of protein bound DNA region and protein-free DNA region.

FIG. 17. Detailed examination of the ionic current the translocation of an individual mDNA/MBD-1x transition through the nanopore. Complex of mDNA/MBD-1x translocates slowly in 10³-10⁴ us with deeper current blocking of −3 nA. In addition to extended and deeper blocking, the event of complex also produces sub-conductance changes during the translocation. Most complex events produced two levels of conductance signatures. 1 and 3 represent complex entering into the nanopore and translocation of region with bulk MBD-1x on methylated-DNA at current blocking Level-2. 2 likely represents translocation of MBD-1x-free region on dsDNA at current blocking Level-1. 4 represents the end of complex translocation. The translocation velocity of mDNA/MBD-1x at state 1 and 3 are relatively longer than at state 2, supporting the strong polymer-pore interactions that slows down the translocation velocity of mDNA/MBD-1x complex.

FIG. 18A. Cross-sectional view of solid-state nanopore and biomolecule transport direction across the nanopore along the bias voltage. FIG. 18B. Representative nanopore electrical current signatures of 90 bp unmethylated dsDNA (left) and hypermethylated dsDNA fully bound with methyl-CpGbinding protein (right). FIG. 18C. Comparison of nanopore transport events between 90 bp unmethylated dsDNA (left) and locally methylated dsDNA bound with a single methyl-binding protein (right). Schematics of 90 bp dsDNA fragments showing FIG. 18D unmethylation, FIG. 18E hypermethylation, and FIG. 18F local methylation. Crystal structures of FIG. 18G bare B-form dsDNA (PDB ID: 1BNA), FIG. 18H methyl-CpG-Binding domain protein bound to a symmetric CpG dinucleotide on dsDNA (PDB ID: 1IG4), and FIG. 18I Kaiso zinc finger protein bound to two symmetric adjacent CpGs on dsDNA (PDB ID: 4F6N).

FIG. 19A. TEM image of a 19 nm nanopore. FIG. 19B. Nanopore current trace of 90 bp unMeth DNA transports at 200 mV driving force. No noticeable events are observed. FIG. 19C. Nanopore current traces show transports of 90 bp hyMethDNA/MBD1x complexes. Data traces from left to right are recorded in a range of driving potential across the membrane, from 150 mV to 350 mV, in increments of 50 mV. Contour plots show transports of hyMethDNA/MBD1x at 250 mV (FIG. 19D) and 300 mV (FIG. 19E). FIG. 19F. Representative single molecule transport events of hyMethDNA/MBD1x complex at various voltages. The number of events used for the analysis is 235 at 150 mV, 252 at 200 mV, 255 at 250 mV, 326 at 300 mV, and 341 at 350 mV. FIG. 19G. Current blockade of complex transports. Each value is obtained by fitting the Gaussian function to a current blocking histogram. The obtained values of current blockades are 2.43±0.05, 3.55±0.06, 5.2±0.04, 7.69±0.08, and 9.51±0.07 nA from 150 to 350 mV. The trend line in short dashes is obtained by fitting first-order polynomial values, indicating an increase of current blocking at higher bias voltages. FIG. 19H. Transport duration of the complex. Each value is obtained by fitting the exponential decay to a transport time histogram. The obtained values of transport duration are 7.96, 4.72, 2.83, 1.43, and 1.06 ms from 150 to 350 mV, and the values are fit well to an exponential decay function as shown in the short dashed trend line, indicating voltage dependency of transport duration.

FIG. 20A. Nanopore current trace shows mixture transports of 90 bp long unMethDNA and hyMethDNA/MBD1x complex, recorded at 300 mV in 1 M KCl containing 10 mM Tris and 1 mM ethylenediaminetetraacetic acid at pH 7.6. FIG. 20B. Scatter plot in gray color shows mixture events of 90 bp long unMeth DNA and hyMethDNA/MBD1x complex and in orange color shows 90 bp long unMethDNA-only events obtained from separate experiment. Separate unMeth DNA-only events match well with fast-shallow current blocking events found in the mixture, indicating that the fast-shallow events of the mixture represent transport of 90 bp unMethDNA. FIG. 20C. Representative sample transports of unMethDNA marked with inverted triangles in FIG. 20A. FIG. 20D. Representative sample transport events of hyMethDNA/MBD1x complex marked with upward pointing triangles in FIG. 20A. FIG. 20E. Current blocking histograms of unMethDNA transports recorded at 250 mV (top) and 300 mV (bottom). FIG. 20F. Transport duration histograms recorded at 250 mV (top) and at 300 mV (bottom). Events obtained from the mixture are in blue, and separate unMethDNA-only are in orange for both FIG. 20E and FIG. 20F. FIG. 20G. Current blocking histogram of hyMethDNA/MBD1x complex transports. FIG. 20H. Transport duration histogram of hyMethDNA/MBD1x complex transports. The histograms are built with prolonged-deep current blocking events in mixture transports, as shown in FIG. 20D, recorded at 250 mV (pink) and at 300 mV (red) for FIG. 20G and FIG. 20H. FIG. 20I. Transport duration values of unMethDNA and hyMethDNA/MBD1x complexes. Each point is obtained by fitting the transport duration histogram to an exponential decay. Transport durations of unmethylated dsDNA are in a range between 100 and 125 μs, while complex transports are in a prolonged duration of 5.59 and 2.86 ms at 250 and 300 mV. FIG. 20J. Current blockade values obtained by fitting the Gaussian function to the current blockings. Current blockade of unMethDNA transports are ˜0.45 nA at 250 mV and ˜0.56 nA at 300 mV, while hyMethDNA/MBD1x complexes block current of ˜2.5 nA at 250 mV and ˜3.5 nA at 300 mV. The number of events used for these analyses was 2135 for the mixture and 841 for separate unMethDNA at 250 mV and 1860 for the mixture and 613 for unMethDNA at 300 mV.

FIG. 21A. Representative nanopore ionic current traces of unMethDNA (concentration at 1 nM) transports. FIG. 21B. Representative sample single-molecule transport events from raw traces of hyMethDNA/MBD1x complex (concentration at 10 pM) transports. FIG. 21C. 90 bp long hyMethDNA/MBD1x complex and unMethDNA transports (unMethDNA events, n=1225 at 150 mV, 1866 at 200 mV, 1136 at 250 mV, 741 at 300 mV, and 436 at 350 mV; hyMethDNA/MBD1x events, n=963 at 250 mV, 943 at 300 mV, 605 at 400 mV, and 848 at 500 mV). FIG. 21D. 60 bp long hyMethDNA/MBD1x complex and unMethDNA (unMethDNA events, n=2135 at 150 mV, 1613 at 200 mV, 1088 at 250 mV, and 787 at 300 mV; hyMethDNA/MBD1x events, n=336 at 200 mV, 503 at 250 mV, 549 at 300 mV, and 505 at 400 mV). FIG. 21E. 30 bp long hyMethDNA/MBD1x complex and unMethDNA (unMethDNA events, n=1167 at 150 mV, 578 at 200 mV, 788 at 250 mV, 681 at 300 mV, and 781 at 350 mV; hyMethDNA/MBD1x events, n=160 at 200 mV, 132 at 250 mV, 198 at 300 mV, and 126 at 400 mV). HyMethDNA/MBD1x complex transports are in brown, and unMethDNA transports are in purple. The values of the current blockade are shown in the left panel, and the values of transport duration are shown in the right panel for FIGS. 21C-21E. The short dashed trend lines for current blockade are obtained by fitting the first-order polynomial, indicating an increased current blockade at higher driving force. The short dashed trend lines for transport duration are obtained by fitting to the exponential decay, indicating voltage-dependent translocation velocity.

FIG. 22A. Side view of crystal structure that describes loMethDNA bound with a single KZF (PDB ID: 4F6N). FIG. 22B. Top-down view of loMethDNA/KZF complex. Dimension of the complex is measured at 4.9 nm from end to end of KZF bound on loMethDNA. FIG. 22C. TEM image of a 5.5 nm diameter nanopore. FIG. 22D. Nanopore ionic current trace of mixture transports between 1 nM of 90 bp long unMethDNA and 10 pM of 90 bp long loMethDNA/KZF complex. FIG. 22E. Representative sample single-molecule transports of unMethDNA and all-point histogram (right, n=50), demonstrating open pore current and current blockade of unMethDNA transports. FIG. 22F. Representative transport events of loMethDNA/KZF complex and allpoint histogram (right, n=20). Current blockades in two obvious levels are observed; shallow blockade is attributed to the dsDNA region and the deeper blockade to the protein—DNA region in complex. FIG. 22G. Transport duration histograms of unMethDNA (in purple) and loMethDNA/KZF complex (in brown). FIG. 22H. Deeper current blockade position profile of complex transport events. The number of events used for this analysis is 7497 for unMethDNA and 379 for loMethDNA/KZF complexes.

FIG. 23A. Electrophoretic mobility shift assay (EMSA) for detecting KZF-90 bp hypoMethDNA interactions using 6% polyacrylamide gel. 90 bp dsDNA contains continuous two symmetric methylated CpGs at the center of the sequence. Both DNA only and DNA-KZF are suspended in 200 mM NaCl at pH 7.6 containing 10 mM Tris, 1 mM ZnCl and 1 mM TCEP. To form complex, 100 nM of DNA is mixed with 100 nM of KZF. Gel image shows shifted sharp band for complex that indicates one single KZF has bound on dsDNA. FIG. 23B. EMSA for detecting MBD1x-90 bp hyperMethDNA interactions using 6% polyacrylamide gel. 90 bp dsDNA contains 10 methylated CpG sites. Both DNA only and DNA-MBD1x complex are suspended in 1M KCl at pH 7.6 containing 10 mM Tris, 1 mM EDTA and 0.4 mM DTT. To form complex, 100 nM of DNA is mixed with 150 nM MBD1x. Gel image shows shifted wider band for complex that indicates slightly differing number of MBD1x bound on dsDNA. Both EMSA use NEB 100 bp ladder and Sybr safe stain dye. Gel image taken by GE Image Quant LAS 4100.

FIG. 24A. TEM image of a typical nanopore of 3.5±0.3 nm in diameter fabricated in 20 nm-thick SiN membrane using a focused electron beam is shown. FIG. 24B. The representative traces show transports of dsDNA through the nanopore. Each spike-like event in nanopore ionic current traces indicates the translocation of a single molecule through the nanopore. The presented data traces are recorded at 150 mV, 300 mV and 500 mV using 10 kHz built-in Bessel low pass filter and 10 μs sampling rate, showing translocation events of 850 bp dsDNA through a ˜3.5 nm nanopore in 600 mM KCl at pH 8.0 (TrisHCl) containing 1 mM EDTA. FIG. 24C. A detailed view of these events showing the key parameters identifying single-molecule transport with current blocking, ΔI, and duration, t_{duration}. FIGS. 240-24E. Typical dsDNA translocation statistics and passage parameter outputs are shown. FIG. 24D. The values of each current blockade for each applied voltage is obtained by fitting the histogram of the blocked current to the Gaussian function, and FIG. 24E the values of current blockade duration are obtained by fitting the translocation duration to an exponential decay function. Previous studies show a linear increase of the current blockade and exponentially reduced translocation duration as a function of applied voltages.^{1,2}

FIG. 25A. Open pore current traces of 19 nm nanopore in 1M KCl at pH 7.6 containing 10 mM Tris and 1 mM EDTA. Current traces are recorded from −200 mV to 200 mV at 20 mV increments. FIG. 25B. TEM image of a nanopore in diameter of 19 nm. Scale bar in image is in 10 nm. FIG. 25C. Current-Voltage characteristic curve (IV curve) recorded in FIG. 24A.

FIG. 26A. Contour plot corresponding to scatter plot of mixture transports events between naked DNA and hyperMethDNA/MBD1x complex in grey color shown in FIG. 20B (n=1860). FIG. 26B. Contour plot corresponding to separate nanopore transport events of naked DNA only shown in orange color in FIG. 20B. (n=613).

FIGS. 27A-27B. Representative transport events of 90 bp hyperMethDNA/MBD1x single-molecule complex through 19 nm (FIG. 27A) and 7.7 nm (FIG. 27B) diameter nanopores. FIGS. 27C-27E. Analysis of single-molecule transports events obtained from 19 nm nanopore are in brown color and 7.7 nm in cyan color. FIG. 27C. Current blockades. The lines are obtained by fitting the current blockade points to 1^{st} order of Polynomial function, indicating linear increase and voltage-independency. FIG. 27D. Transport duration. The lines are obtained by fitting the transport time points to Exponential decay function, indicating voltage-dependency of transport velocity. FIG. 27E. Occurrence of single-molecule transports at the function of applied voltages. The lines are obtained by fitting the occurrence to Exponential function, indicating voltage-dependent occurrence.

FIG. 28A. Current trace of 90 bp-long unmethylated dsDNA transports through 7.7 nm nanopore. The trace is recorded at applied voltage of 300 mV in 1M KCl 10 mM Tris 1 mM EDTA titrated at pH 7.6. FIG. 28B. Typical parameters of interest for investigating electrical signature produced by transports of single molecule. ΔI, the current blocking by single-molecule transport, is between open pore current, I_{O}, and blocked current, I_{B}, and t_{D} is the transport duration of single molecule via the nanopore. FIG. 28C. Scatter plot of 90 bp-long unmethylated dsDNA transports at various applied voltages. FIG. 28D. Transmission electron microscope (TEM) image of 7.7 nm nanopore with a scale bar of 5 nm. FIG. 28E. Current-Voltage characteristic at the function of applied voltages in 1M KCl at pH 7.6 containing 10 mM Tris and 1 mM EDTA. FIG. 28F. 90 bp-long unmethylated dsDNA transport duration at various applied voltages. The each value was obtained by fitting the transport duration histogram to the exponential function. FIG. 28G. Current blocking of the 90 bp-long unmethylated dsDNA transport. Each point was obtained fitting blocked current histogram to the Gaussian function.

FIG. 29. Current blockade histogram for naked DNA (top) is built with 7497 events in purple color and complex (bottom) is built with 379 events in wine color.

FIG. 30. Duration of deeper current blockade. The deeper current blockade from entire hypoMethDNA/KZF transport events is separately measured and fitted to exponential decay function to obtain duration of deeper current blockade. The duration of deeper current blockade obtained for 0.33 ms and -400 events contributes to the histogram.

FIGS. 31A-31C. Nanopore experiment for KZF only in 200 mM NaCl at pH 7.6. FIG. 31A. Transmission electron microscopy (TEM) image of a nanopore in diameter of 5.5 nm. FIG. 31B. Nanopore open pore current trace before KZF is introduced. FIG. 31C. Nanopore current trace after 100 pM of KZF is introduced. KZF is introduced to the cis side and positive bias voltage is applied to trans side. No noticeable transport through the nanopore is observed.

FIGS. 32A-32B. Comparison of nanopore current traces recorded in 200 mM NaCl pH 7.6. FIG. 32A. Nanopore current trace shows transport of mixture between 90 bp unMeth DNA and 90 bp hypoMethDNA/KZF complex. FIG. 32B. Nanopore current trace shows transport of mixture between 90 bp unMeth DNA and KZF. The data trace shows only shallow current blockade translocation events, indicating no simultaneous translocation of unMeth DNA-protein or overlapped two DNA.

FIG. 33. Heparin column purification of Kaiso protein is shown. Fluorescent protein fused Kaiso protein is cleaved with thrombin to remove fused fluorescent protein, followed by thrombin clean-up by streptavidin column. The resulting mixture of mCherry and Kaiso is separated by heparin column. Briefly, Kaiso is bound on heparin column, but not the fluorescent protein is cleaned up and wasted to flow through. Polished protein is shown in SDS-PAGE.

FIG. 34. SDS-PAGE of Kaiso purification. Kaiso is recovered from cell pellet with 8M Urea in denaturing condition, followed by slow refolding on Ni-NTA column overnight at 4° C. After thrombin cleavage on Ni-NTA column overnight, protein is eluted with 1M imidazole and cleaved protein mixture is purified with Streptavidin column to clean up biotinlyated thrombin and Kaiso was polished with heparin column to remove mCherry protein mixture.

FIG. 35. Process flow shows analysis of methylation profile in a stool DNA sample using nanopore-based sensor, in comparison with conventional assay using bisulfite conversion.

FIG. 36A. TEM image of 3 nm nanopore fabricated in SiN membrane. Scale bar is in 5 nm. FIG. 36B. DNA translocation through the nanopore under an applied voltage. FIG. 36C. Electrical current signatures resulting from DNA translocations. Each downward spike-like event represents transport of single molecule. FIG. 36D. An expanded view of a single translocation event.

FIG. 37A. Representative MBP bound methDNA transports and FIG. 37B unmethylated DNA. FIG. 37C Crystal structure of unmethylated DNA and FIG. 37D MBD1x bound methDNA. FIG. 37E TEM image of 8 nm nanopore fabricated in SiN membrane. FIG. 37F representative current signature of KZF bound DNA transports and FIG. 37G current trace shows mixed transports events of unmethylated DNA and KZF bound methDNA. FIG. 37H crystal structure of KZF bound methDNA.

FIG. 38A. Cross-sectional view of label-free methylated DNA transport through SFN. Fast unmethylated DNA transport (FIG. 38B) vs. longer transport duration expected for methylated DNA (FIG. 38C) due to the highly specific binding between methyl groups and anti-5mC antibodies.

FIGS. 39A-39B. Hybridization of fully complementary probe with FIG. 39A unmethylated target DNA fragment and with FIG. 39B methylated fragment. MBD1x does not bind to asymmetric methylation on DNA, but does bind to symmetric methylated CpG dinucleotides. Other methylation binding proteins can bind to hemi-methylated DNA.

FIG. 40. 1. Qiagen beads with dsDNA in high salt solution introduced into a microfluidic channel and concentrated using magnetic forces (DEP forces) in over the nanopore area, 2. MBD1x proteins introduced into the channel. 3. MBD1x and dsDNA on beads react to form complex. 4. UDG (Uracil-DNA glycosylase) is introduced to cut the DNA-protein complex from beads. 5. Apply voltage and run the DNA-protein complex through the nanopore. 6. Distinguish methylated and unmethylated dsDNA with MBD1x.

FIG. 41A. Nanopore-based sensor integrated with PDMS microfluidic-channel. FIG. 41B. Magnetic-force driven beads collection (dotted circle) without patterned magnetic layers within the channel. FIG. 41C Chamber with patterned magnetic layers as micro-magnetics to allow for a uniform bead distribution (dotted circle). FIG. 41D. Close-up view of nanopore sensing region marked in red rectangle in FIG. 41A. The dot depicts a nanopore and pattern squares are the magnets. FIG. 41E. A single molecule of methDNA/MBD complex on a bead via four uracils. FIG. 41F. UDG cutting four uracils between amine terminal and complementary probe. FIG. 41G. MBP bound methDNA is release from the beads. FIG. 41H. Nanopore detection of MBP bound methDNA (upper) and asymmetric DNA (lower). Schematic depicts asymmetric DNA transport through nanopore (FIG. 41I) and MBP bound methDNA transport (FIG. 41J).

FIG. 42. Nanopore through a dielectric membrane of SiN; 10 nm-thick 18 nm×18 nm.

FIG. 43. Schematic illustration of a system for capturing, concentrating and biomarker introduction to polynucleotides of interest, followed by nanopore transit for biomolecular characterization of the polynucleotide transiting the nanopore.

FIG. 44. Schematic illustration of a system that captures polynucleotide of interest and mixes biomarker, with subsequent introduction to a nanopore for biomolecular characterization.

FIG. 45. Embodiment of a system with on-chip mixing, concentrating around the nanopore and molecular parameter characterization by transit through the nanopore. The capturing and/or concentrating around the nanopore is compatible with a number force-inducing means, such as magnetic, electrical, fluidic mass transport, selective binding and any combinations thereof.

FIGS. 46A-46B. Nanopore ionic current traces recorded at 200 mV in 1M KCl at pH 7.6. FIG. 46A. Nanopore assay detects uDNA at 100 pM but all events are not reliably detectable. FIG. 46B. Mixture of uDNA and mDNA:biomarker complex is detected through nanopore. Complexed mDNA:biomarker events are detected via a significantly noticeable current blockade and translocation duration.

FIG. 47. Current blockade and translocation duration for various applied potential.

FIG. 48: Sequence ID Nos. and related descriptions, including of target DNAs. The target dsDNA fragments are purchased from IDTDNA, and various length of 90 bp, 60 bp, and 30 bp fragments are synthesized. Hypermethylated dsDNA consists of 10% of methylated CpGs, proportional to its entire length and uniformly distributed through entire sequence. Methylated CpGs are underlined and 5-carbon methylated cytosine is colored in green. 30 bp DNA fragment has 3 symmetrically methylated CpG dinucleotide, 60 bp for 6 methylated CpGs, and 90 bp for 10 methylated CpGs. Hypomethylated dsDNA fragments are designed to have thirty potential CpG methylation sites, only symmetric two adjacent methylated CpGs at the center activated, and repeated sequence of nine CGACGT. DNA fragments holding none methylations are prepared to pair the complementary experiment of hypermethylation vs. unmethylation.

FIG. 49 Estimated number of new cancer cases and deaths by sex for colorectal and pancreatic cancers, US, 2014. (Based on data in Cancer Facts & Figures.¹)

FIG. 50: Cancer detection in plasma/serum by DNA methylation markers.²

### DETAILED DESCRIPTION OF THE INVENTION

In general, the terms and phrases used herein have their art-recognized meaning, which can be found by reference to standard texts, journal references and contexts known to those skilled in the art. The following definitions are provided to clarify their specific use in the context of the invention.

“Polynucleotide” is used broadly herein and includes, for example, DNA, RNA, oligonucleotides, and combinations thereof, and may be single stranded or double stranded. The polynucleotide may be naturally occurring or may be engineered or synthetic. A “biomolecular parameter” refers to a measurable or quantifiable property of the polynucleotide. The parameter may be a constant, or a yes/no state, such as the sequence or a sequence portion. The parameter may vary for a particular biomolecule depending on the state or conditions of the biomolecule, such as for a biomolecular parameter that is a methylation state, binding event and/or secondary structure. An “electrical parameter” refers to a parameter that can be electrically measured or determined and that relates to the biomolecular parameter. Of particular relevance herein, are electrical parameters used to monitor a passage parameter output. “Passage parameter output” refers to a measurable or calcutable variable that reflects passage of a polynucleotide through the nanopore, and tends to be derived from or may relate to the electrical parameter. Examples include blockade current, threshold voltage, transit time, transit velocity, resistance, conductance, and statistical parameters thereof. The process parameter is described as an output to reflect that it it can be measured or determined and that it may be temporally varying.

“Polynucleotide of interest” refers to a portion of a longer polynucleotide, or a smaller fragment thereof, that contains information about a desired biomolecular parameter. For example, a specific portion of DNA may contain information about a genetic mutation state (mutation present or absent), methylation state (e.g., level or pattern of methylation), or other factor that can be measured herein. Those specific portions may be contained within a specific fragment, so that other fragments not of interest are present. Advantages of the instant invention include the ability to precisely locate the polynucleotides of interest to the region of the nanopore of interest, for subsequent high-quality analysis and characterization, without any corresponding increase in concentration of polynucleotides not of interest. Without this important aspect, there is a risk of loss of desirable signal in the noise of the overwhelming number other polynucleotides that may be irrelevant for the application on hand. Polynucleotide of interest, for clarity, are those polynucleotides that may or may not have a biomolecular parameter of interest, but that are to be studied so as to characterize the biomolecular parameter of interest.

“Methylation” refers to DNA having one or more residues that are methylated. For example, in all vertebrate genomes some of the cytosine residues are methylated. DNA methylation can affect gene expression and, for some genes, is an epigenetic marker for cancer. Two different aspects of DNA methylation can be important: methylation level or content as well as the pattern of methylation. “Methylation state” is used broadly herein to refer to any aspect of methylation that is of interest from the standpoint of epigenetics, disease state, or DNA status and includes methylation content, distribution, pattern, density, and spatial variations thereof along the DNA sequence. Methylation detection and parameter characterization via nanopores is further discussed in U.S. Pat. Nos. 8,394,584, 8,748,091 and 2014/0174927.

In addition, biomolecular parameter refers to a quantitative variable that is measurable and that can be reflected by the polynucleotide transit through a nanopore, such as for example, translocation speed through a nanopore, variations in an electrical parameter (e.g., changes in the electric field, ionic current, resistance, impedance, capacitance, voltage) in the nanopore as the polynucleotide enters and transits the pore, including temporary or transitory interactions between the polynucleotide and a nanopore surface region functionalized with a chemical moiety.

“Dielectric” refers to a non-conducting or insulating material. In an embodiment, an inorganic dielectric comprises a dielectric material substantially free of carbon. Specific examples of inorganic dielectric materials include, but are not limited to, silicon nitride, silicon dioxide, boron nitride, and oxides of aluminum, titanium, tantalum or hafnium. A “high-k dielectric” refers to a specific class of dielectric materials, for example in one embodiment those dielectric materials having a dielectric constant larger than silicon dioxide. In some embodiments, a high-k dielectric has a dielectric constant at least 2 times that of silicon dioxide. Useful high-k dielectrics include, but are not limited to Al₂O₃, HfO₂, ZrO₂, HfSiO₂, ZrSiO₂ and any combination of these. In an aspect, any of the methods and devices provided herein have a dielectric that is Al₂O₃.

“Conductor-dielectric stack” refers to a plurality of layers, with at least one layer comprising an electrical conductor and another layer a dielectric. In an embodiment, a layer may be geometrically patterned or deposited, such as in a nanoribbon configuration including a conductor layer that is a conducting nanoribbon having a longitudinal direction that is transverse to the passage formed by the nanopore. In an aspect, the stack comprises 2 or more layers, 3 or more layers, or a range that is greater than or equal to 5 layers and less than or equal to 20 layers. In an aspect, adjacent conductor layers are separated from each other by a dielectric layer. In an aspect the outermost layers are conducting layers, dielectric layers, or one outermost layer that is dielectric and the other outermost layer at the other end of the stack is a conductor. In an aspect, local electric field may be applied and controlled near the membrane surface by selectively patterning a dielectric layer that covers an underlying conductor layer that is electrically energized. Any of the methods and devices provided herein have a conducting layer that is grapheme. As exemplified herein, the term graphene can be replaced, as desired, with other atomically thin electrically conducting layers, such as MoS₂, doped silicon, silicene, or ultra-thin metal.

“Fluid communication” or “fluidly connects” refers to a nanopore that permits flow of electrolyte, and specifically ions in the electrolyte from one side of the membrane (e.g., first fluid compartment) to the other side of the membrane (e.g., second fluid compartment), or vice versa. In an aspect, the fluid communication connection is insufficient to readily permit polynucleotide transit between sides without an applied electric field to facilitate transit through the nanopore. This can be controlled by combination of nanopore geometry (e.g., diameter), nanopore surface functionalization, applied electric field through the nanopore and polynucleotide and fluid selection.

“Specific binding” refers to an interaction between two components wherein one component has a targeted characteristic. Binding only occurs if the one component has the targeted characteristic and substantially no binding occurs in the absence of the targeted characteristic. In an embodiment, the targeted characteristic is a nucleotide type (e.g., A, T, G, C), an amino acid, or a specific sequence of nucleotides, chemical change of one or more nucleotides, such as oxidation, methylation, or the like.

Unless described otherwise, “adjacent” refers to a relative position between components that permit a functional and beneficial interaction between the components. For example, a position may be functionally described as adjacent to a nanopore entrance. This refers to positions that result in an desired interaction with the nanopore entrance, such as the ability to enter the nanopore under an applied electric field. Alternatively, adjacent may refer to an absolute dimension, such as within 500 μm, within 250-μm, or within 100 μm.

The invention may be further understood by the following non-limiting examples. All references cited herein are hereby incorporated by reference to the extent not inconsistent with the disclosure herewith. Although the description herein contains many specificities, these should not be construed as limiting the scope of the invention but as merely providing illustrations of some of the presently preferred embodiments of the invention. For example, the scope of the invention should be determined by the appended claims and their equivalents, rather than by the examples given. US 2014/0174927 is specifically incorporated by reference to the extent not inconsistent herewith for the systems, devices and methods provided therein as related to biomolecular characterization by transit of the biomolecule through a nanopore under an applied electric field.

Oxidative mechanisms in DNA and RNA are known to contribute to the initiation, promotion, and progression of disease. A list of oxidative modifications to DNA are outlined herein and summarized by Cooke et al. FASEB J. 2003; 17:1195-1214. The systems provided herein facilitate detection of a variety of these modifications through selective binding of an antibody and monitoring a passage parameter output during nanopore transit.

Oxidative DNA damage or DNA lesions including 8-OH-dG are established biomarkers of oxidative stress and coupled with their mutagenicity in mammalian cells, this has led to their proposed use as biomarkers in diseases such as cancer. For example, significantly higher levels of 8-OH-dG in tumor vs. non-tumor tissue was observed in primary breast cancer, elevated levels of 8-OH-dG in tumor tissue compared to normal mucosa in colon cancer, and lymphocyte DNA lesion levels significantly elevated in acute lymphoblastic leukemia (8-OH-Gua, 8-OHAde, 5-OH-Cyt) vs. controls.

Many oxidative base lesions are mutagenic. For example, 8-OH-dG has mutation frequencies of 2.5-4.8% in mammalian cells and for the most part, 8-OH-dG formed in situ results in G→T substitutions; alternatively, 8-OH-dGTP may be misincorporated opposite dA, producing an A→C substitutions. DNA oxidative damage also affects expression in other ways, for example, by altering DNA conformation during replication and transcription, preferential repair of certain oxidative subtypes and microsatellite instability in the promoters of various genes. Examples of this include reduced activities of the antioxidant enzymes catalase, glutathione peroxidase, and superoxide dismutase, with concomitant increased levels of oxidative DNA damage, as reported in acute lymphoblastic leukemia. GC3TA transversions potentially derived from 8-OH-dG have been observed in vivo in the ras oncogene and the p53 tumor suppressor gene in lung and liver cancer.

Detection of all of these different types of DNA and RNA modifications with a nanopore coupled with up-stream sample preparation described herein are applications compatible with any of the methods and systems provided herein.

Detection of Epigenetic Modifications: Demonstrated herein is detection of 5-methylcytosine (5mC). Genomic DNA, of course, contains other forms of modified cytosines, such as 5-hydroxymethylcytosine (5hmC), 5-formylcytosine (5fC), and 5-carboxylcytosine (5caC), hemi-methylated DNA. All of these act as epigenetic marks that regulate gene expression. The nanopore based methylation detection assay described herein is compatible with any of these markers, with detection of 5mC being but one specifically exemplified embodiment.

5hmC exists as an independent epigenetic mark, as a potential demethylation intermediate product from 5mC in certain types of neurons and embryonic stemcells and as an intermediate oxidation state in the formation of 5-formylcytosine (5fC) and 5-carboxylcytosine (5caC). Thus the detection of these different states in single genes may be useful as a tool for monitoring progression or changes in DNA.

Weng et al. (Neurotherapeutics (2013) 10:556-567) suggest that neurons exhibit 5-10 times higher levels of 5hmC than other somatic tissues. Substantial increases in brain 5hmC levels with aging have also been observed. Together, these findings indicate that 5hmC may play a role in neurodevelopment, as well as pathogenesis of neurodegenerative diseases.

Families of proteins exist that bind to epigenetic modifications to translate this information into downstream biological processes. In mammals there are 3 families of methylCpG binding proteins that recognize methylated DNA: the Uhrf family, the methylCpG binding domain (MBD) family, and the Kaiso family. ZBTB4 and ZBTB38 are capable of recognizing a single methylated CpG dinucleotide. The methods and systems provided herein are compatible with any of these families, as well as any targeted antibody.

Pharmacogenetics and Monitoring Drug Response: Systems and methods provided herein can be used to monitor the demethylation status of specific genes in response to a variety of drug treatments, such as Decitabine. To date, a number of pharmacological agents that modify the epigenome—including inhibitors of DNA methylation and various histone modifications—have been developed and used both for research and the clinic. This is described further in Weng et al. Response to those drugs in terms of monitoring the binding to catalytic sites of DNMTs, hence preventing the formation of the DNA-protein complex can be studied. Note, 5-Aza and 5-Daz are nucleotide analogs of cytidine. Mechanistically, it is believed that 5-Aza and 5-Daz must be integrated into genome before irreversibly binding to the catalytic sites of DNMTs.

Detection of SNPs and Sequence Modifications: A number of proteins look for specific sequences on DNA (motifs) that they recognize before binding at that location. For example, Kaiso and ZBTB4 can recognize the consensus Kaiso binding site, TCCTGC, while ZBTB38 binds to the CACCTG E-box motif. These notable differences in DNA binding preferences indicate that generic protein-DNA binding interactions can be studied using the present nanopore-based platform to look for single nucleotide polymorphisms in these binding domains, which in certain cases correlate with specific diseases.

Detecting RNA Modifications (see Lee et al. Cell 158, Aug. 28, 2014, 980-987). Through the nanopore assay, we can detect modifications to RNA. Abundant noncoding RNAs such as rRNAs and tRNAs are extensively modified, whereas mRNA modifications are thought to be relatively low in frequency apart from the common terminal modifications, m₇G cap and poly(A) tail. The most abundant internal modification on mRNA is N₆-methyladenosine (m₆A). Studies have revealed that the methylation status of some m₆A sites dynamically changes in stress conditions, implicating a potential role of m₆A in stress responses, preventing mRNA decay and disease. A number of approaches have been taken up to detect m₆A. e.g. Methylation of a specific site can be quantitated by a digestion-based method called SCARLET as well as RNA seq. Through a nanopore assay of the instant invention, we can detect m₆A by binding to specific proteins. For example, FTO is an m₆A demethylase implicated in the dynamic and reversible nature of m₆A modification with a binding domain to this modification. The YTH domain family is widespread in eukaryotes and is known to bind to ssRNA through the YTH domain. Though all YTHDF1-3 show selective binding to m₆A embedded in consensus sequences, YTHDF2 has the highest affinity.

### Example 1

### Detection and Quantification of Methylation in DNA Using Solid-State Nanopores

Epigenetic modifications in eukaryotic genomes occur primarily in the form of 5-methylcytosine (5 mC). These modifications are heavily involved in transcriptional repression, gene regulation, development and the progression of diseases including cancer. Provided herein is a new single-molecule assay for the detection of DNA methylation using solid-state nanopores. Methylation is detected by selectively labeling methylation sites with MBD1 (MBD-1x) proteins, the complex inducing a 3-fold increase in ionic blockage current relative to unmethylated DNA. Furthermore, the discrimination of methylated and unmethylated DNA is demonstrated in the presence of only a single bound protein, thereby giving a resolution of a single methylated CpG dinucleotide. The extent of methylation of a target molecule can also be coarsely quantified using this novel approach. This nanopore-based methylation sensitive assay circumvents the need for bisulfite conversion, fluorescent labeling, and PCR and is, therefore, very useful in studying the role of epigenetics in human disease.

DNA methylation is one of the most important and frequently occurring epigenetic modifications in mammalian cells and plays an essential role in regulating cell growth and proliferation. In humans, the most common epigenetic modification of DNA involves the addition of a methyl group at the 5-carbon position of cytosine (5-methylcytosine or 5 mC), which occurs exclusively at symmetric CO sites on the DNA double helix and are referred to as CpG dinucleotides. Hypermethylation of the promoter sequences of various genes has generally been associated with transcriptional repression through mechanisms such as the recruitment of methylated CpG binding proteins (MBDs), histone deacetylation and chromatin remodeling^{1,2}. Furthermore, aberrant methylation in the promoter sequences of various genes can point to specific pathways disrupted in almost every tumor type including cancers of the prostate, breast, head and neck, lung and liver, whilst correlating with disease severity and metastatic potential^{3,4,5,6,7,8}. In fact, the tumor prevalence of many methylation markers is considerably higher than that of genetic markers⁴; one example being the hypermethylation of CpG dinucleotides in the promoter sequence of the glutathione S-transferase pi (GSTP1) gene and is observed in over 90% of prostate cancer patients⁹. Methylation analysis will therefore likely play a pivotal role in the diagnosis and treatment of such diseases.

Interestingly, cancer-specific methylated DNA from most tumor types is present in biopsy specimens and also exist at very low concentrations in the form of free-floating DNA shed by apoptotic cancer cells⁴. Current genome-wide methylation analysis techniques rely on bisulfite genomic sequencing¹⁰ (bisulfite conversion of DNA, PCR amplification and DNA sequencing) and typically require large sample volumes due to DNA degradation during bisulfite conversion¹¹, can exhibit low amplification efficiency and PCR bias¹², and are labor intensive. Targeted methods involving analysis at specific loci or groups of genes such as methylation specific PCR (MSP)¹², MethyLight^{13,14} and DNA microarrays¹⁵ overcome the need for sequencing but still rely on bisulfite conversion, amplification and complex probe design. Therefore, a bisulfite free, amplification free method capable of rapidly and accurately determining the methylation status of panels of genes from minute clinical sample volumes could be of tremendous clinical value.

This example demonstrates a new single molecule assay for determining the methylation status of DNA using solid-state nanopores. Nanopores use the principle of ionic current spectroscopy to electrically interrogate individual DNA molecules with the sensitivity to discern subtle structural motifs^{16,17}. Fabrication of these devices typically involves the physical sputtering of a single nanometer sized aperture in a dielectric membrane using a focused electron beam^{18,19}. The electrophoretic transport of biomolecules through these nano-scale pores has enabled the study of various biophysical phenomena at the single molecule level²⁹, with potential applications in DNA sequencing and medical diagnostics^{16,21,22,23.} (For reviews of nanopore research, see refs 24,25,26,27,28,29,30) Recently, methylated and unmethylated DNA has been examined optically in nanofluidic channels using fluorescently labeled proteins bound to the methylation sites^{31,32}. Nanopore-based ionic current spectroscopy, however, is ideal for single molecule epigenetic analysis eliminating the need for optical measurements. Using nanopore based ionic current spectroscopy, the differentiation of methylcytosine from cytosine has previously been demonstrated by passing these individual nucleotides through a biological nanopore³³, requiring an exonuclease based cleaving of the bases from the original molecule. To date ionic current measurements obtained using a solid-state nanopore, have yet to differentiate methylated from unmethylated single molecules of DNA^{34,35}.

Herein, we demonstrate the electrical discrimination of unmethylated and methylated DNA using solid-state nanopores. Our technique does not require bisulfite conversion, sequencing or fluorescent tags but rather relies on the detection of methylated CpG dinucleotides in DNA by labeling with a 75 amino acid region of the methyl DNA binding protein MBD1, which includes a his-tagged single DNA binding domain and will hereafter be referred to as MBD-1x. The translocation of the methylated DNA—MBD-1x complex through a solid-state nanopore induced approximately a 3-fold increase in the measured blockage current relative to unmethylated DNA. The binding of a single MBD-1x protein to a methylated DNA fragment was sufficient for differentiation with high fidelity, thereby enabling single CpG dinucleotide sensitivity. Methylation could also be coarsely quantified based on the number of bound MBD-1x proteins per molecule, characterized by distinct timescales in the event translocation time histograms. As a result, this amplification- and fluorescent label-free, single molecule assay can be significantly useful in the rapid screening of epigenetic biomarkers for the early detection of diseases such as cancer.

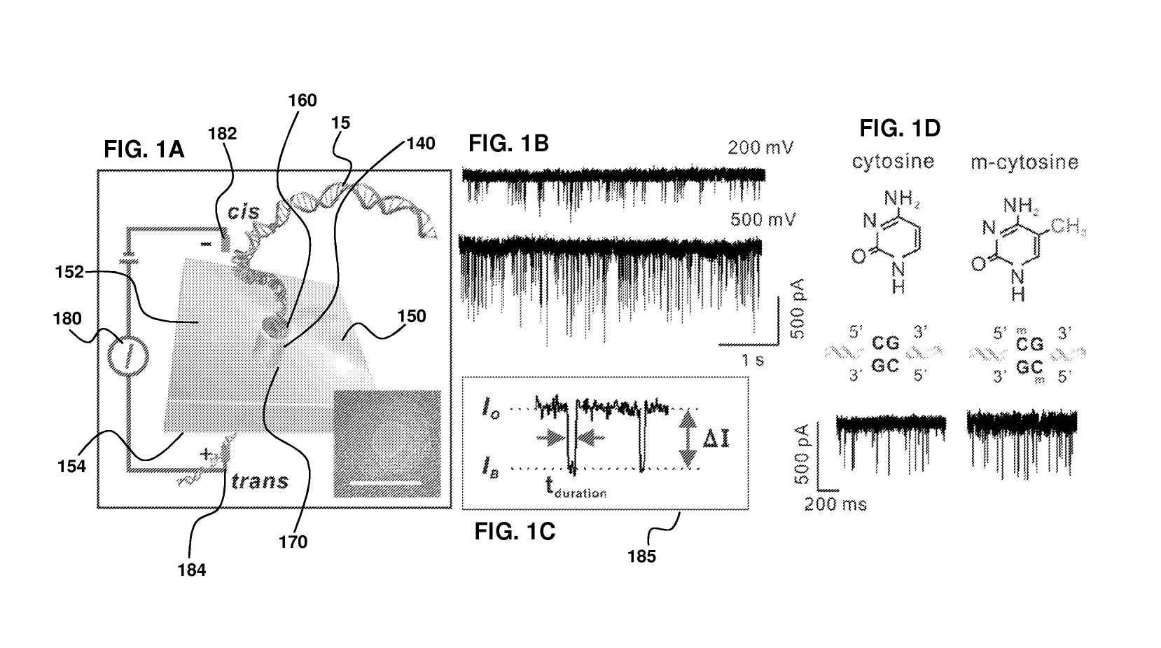

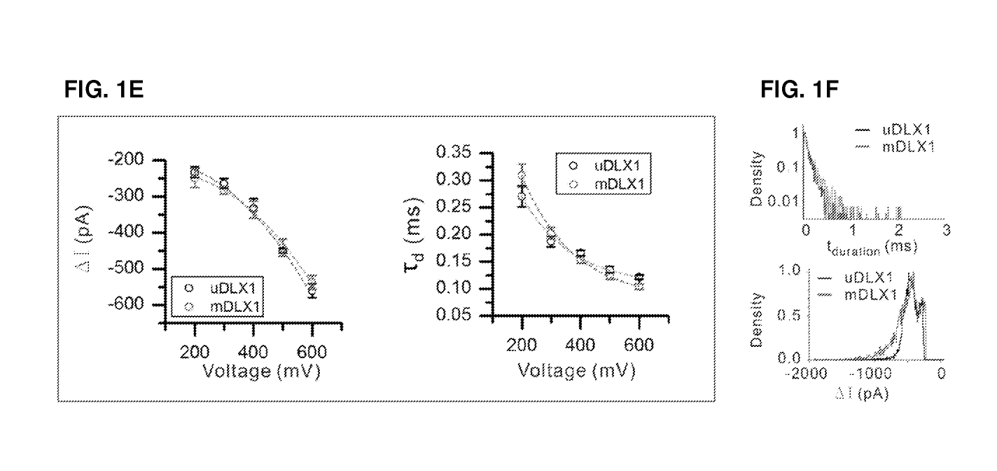

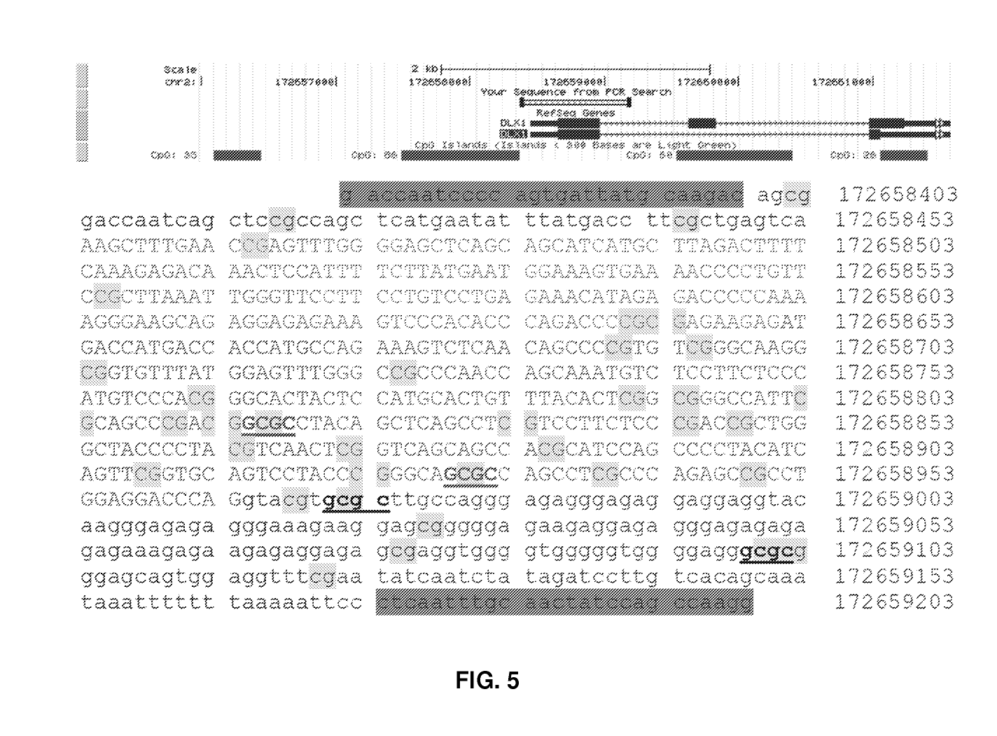

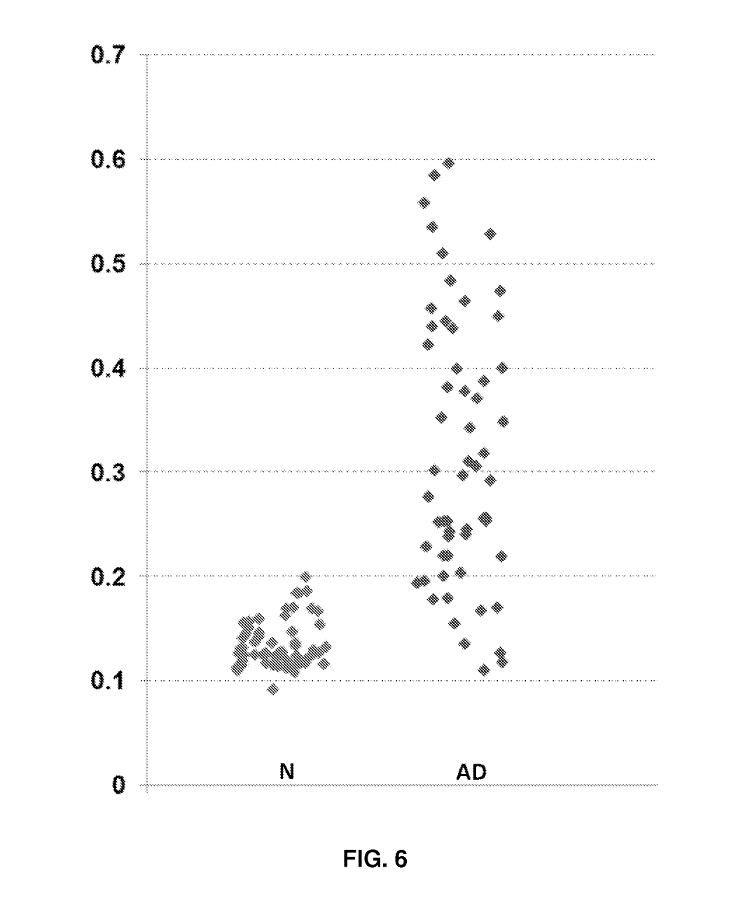

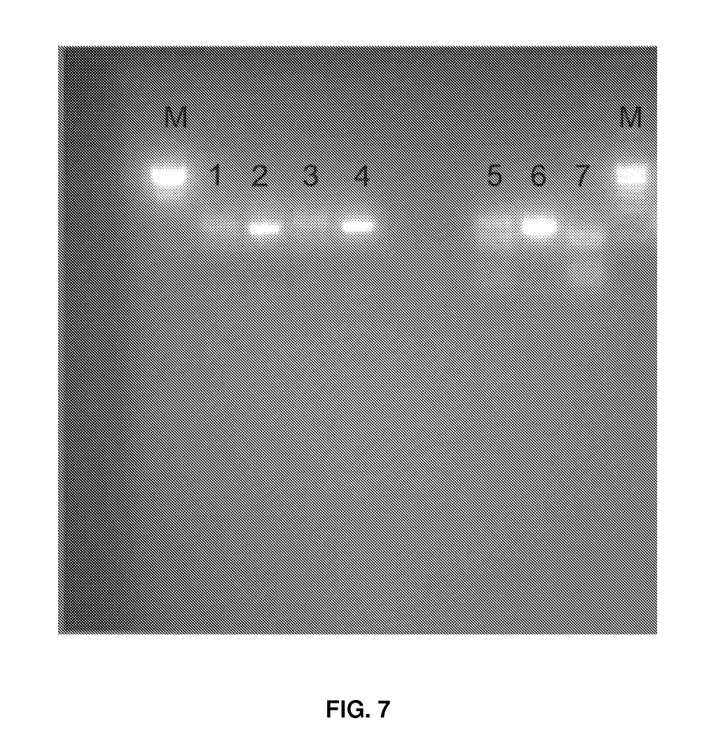

Detection of unlabeled methylated and unmethylated DNA: The electrophoretic transport of double stranded DNA (dsDNA) through a solid-state nanopore is illustrated in the schematic of FIG. 1A, the inset showing a transmission electron microscope image of a 4.2 nm diameter pore. The detection of unmethylated and methylated dsDNA in the absence of MBD-1x is performed using a 4.2 nm pore fabricated in 20 nm-thick SiN membranes according to methods described previously^{18,19}. Briefly, DNA is introduced into the cis chamber. A positive voltage is applied to the trans side resulting in the passage of dsDNA through the nanopore to the trans side. The target fragment used in these studies is an 827 bp region of DLX1 (see FIG. 5 for sequence information), a homeobox gene associated with forebrain development³⁷. Aberrant methylation of DLX1 has been reported in several cancers, including lymphoma³⁸, and brain tumors³⁹. Furthermore, analysis of publicly available methylation profiling data⁴⁰ identified significant hypermethylation of DLX1 promoter in lung adenocarcinomas (see FIG. 6). Therefore, methylated DLX1 promoter has potential clinical utility in cancer diagnosis. This 827 bp DLX1 region contained 36 CpG dinucleotides, which were methylated in-vitro using the M.Sssl DNA methyltransferase. The methylation of the dsDNA was confirmed using the restriction enzyme Hhal (see FIG. 7). Methylated DLX1 will hereafter be referred to as mDLX1 and unmethylated DLX1 will be referred to as uDLX1. Ionic current traces produced by the electrophoretic transport of mDLX1 through the nanopore at various voltages are shown in FIG. 1B, each downward current pulse indicative of the passage of a single mDLX1 molecule though the nanopore. A magnified view of these events is presented in FIG. 1C, the key parameters of interest being the blockage current, ΔI, induced by the passage of the molecule through the pore and the event duration or translocation time, τ_{d}. Chemical structures of cytosine and methylated-cytosine, schematics of CpG dinucleotides in unmethylatd dsDNA and methylated dsDNA, data traces of uDLX1 and mDLX1 recorded at 300 mV are presented in FIG. 1D.

FIG. 1E compares the translocation properties (ΔI and t_{duration}) of methylated and unmethylated DLX1 through the nanopore as a function of applied voltage. Each data point on these plots consists of over 1167 separately recorded DNA translocation events. Voltage-dependent transport of both mDLX1 and uDLX1 is observed; step increases in the applied voltage resulting in higher electrophoretic forces on the molecule and therefore shorter translocation times through the pore^{41,42}. As seen in FIG. 1E, the single molecule sensitivity of a solid-state nanopore alone is not sufficient to distinguish methylated from unmethylated DNA with any statistical significance. This is reiterated by the similar τ_{d} and ΔI histograms (FIG. 1F), each distribution containing over 2153 translocation events recorded at 500 mV. Notably, the time constants for mDLX1 and uDLX1 obtained by exponential fitting to the translocation time histograms of FIG. 1F are within 10% of each other (τ_{M}=0.124±0.006 ms, τ_{U}=0.135±0.006 ms), confirming the inability to consistently distinguish methylated from unmethylated DNA. This result is not surprising given the subtle structural and chemical differences that exist between 5-methylcystosine and cytosine (FIG. 1D). We therefore conclude that these differences along with reported differences in the nanomechanical properties of methylated versus unmethylated DNA³⁴, are not sufficient to give rise to detectable differences in their respective ionic current signatures. This is consistent with previous findings³⁵ and reiterate the need for a methylation specific label in nanopore based methylation studies.

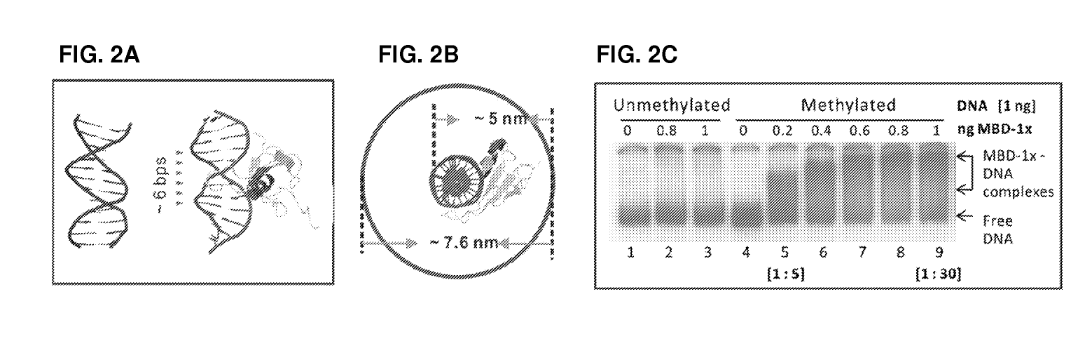

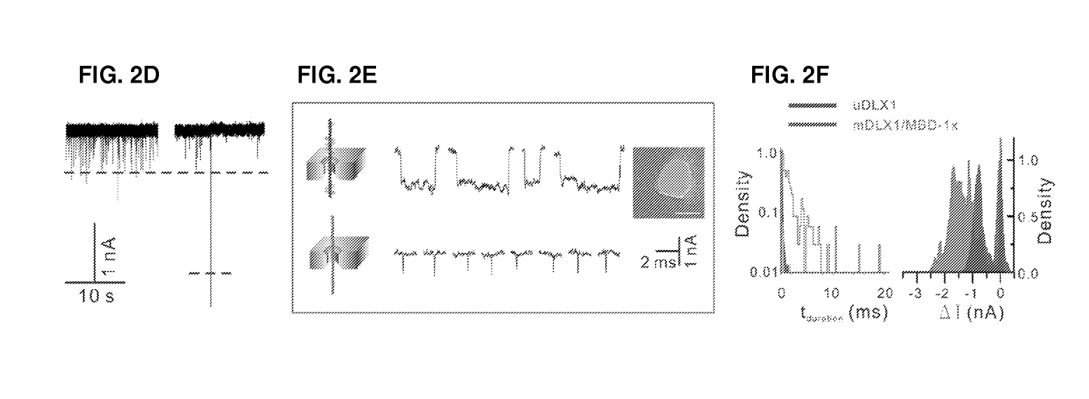

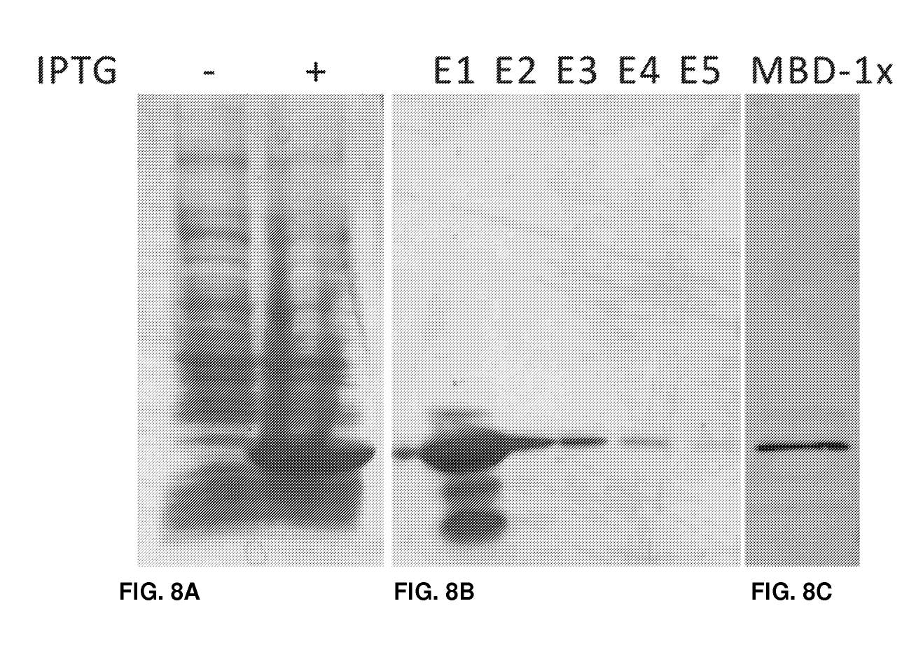

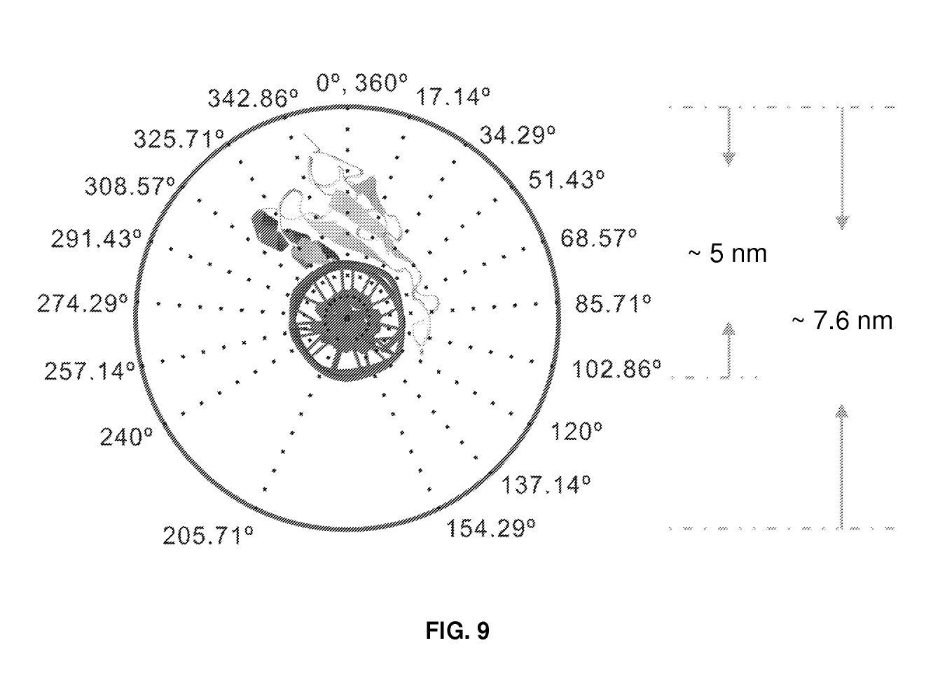

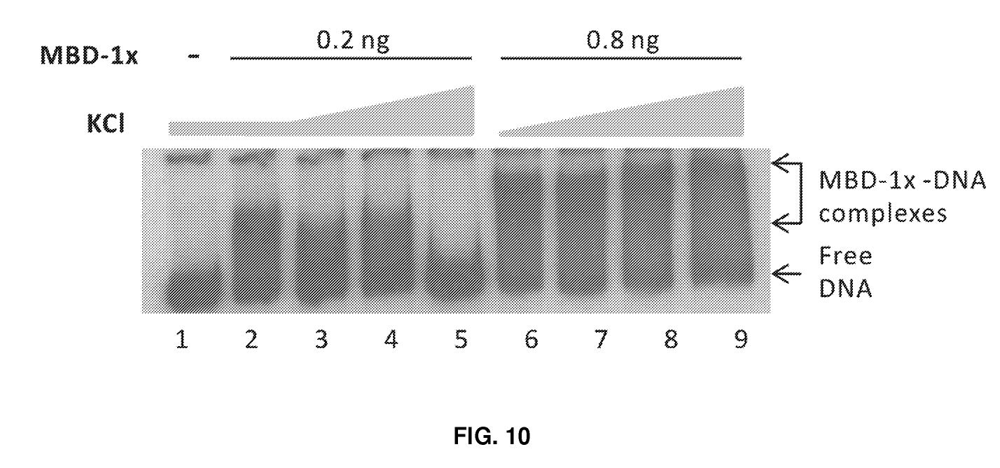

Formation of DNA/MBD-1x Complex: To specifically label methylated DNA, we use the 75 amino acid methylated DNA binding domain of the protein MBD1. MBD1 plays an important role in gene silencing by recruiting AFT71P, which in turn recruits factors such as the histone methyltransferase SETDB1 and is essential in histone deacetylation and transcriptional repression in vertebrates⁴³. Importantly, MBD1 binds symmetrically to methylated but not unmethylated CpG dinucleotides with high affinity⁴⁴ and specificity⁴³. The 75 amino acid MBD-1x is expressed in E. coli and protein purity verified using Coomassie stained gels and Western blot analysis (FIG. 8A-8C). FIG. 2A illustrates the crystal structures of typical B-form dsDNA and the methylated-DNA/MBD complex^{29,30}. X-ray diffraction and NMR spectroscopy confirm that the binding domain of MBD1 occupies ˜5-6 bp in the major groove of the dsDNA helix upon binding to a single methylated CpG dinucleotide^{45,46}. It is therefore likely that only 21-25 of the 36 methyl-CpG sites in the DLX1 probe used here will serve as functional binding sites for MBD-1x, as only these regions contain sufficient spacing between sites to physically accommodate the protein. The relatively small occlusion area of MBD-1x (5-6 bps) also makes this protein ideal for nanopore based methylation analysis. Other MBD family proteins such as MBD2 and MeCP2 are known to protect 12-14 bp around a single binding site⁴⁷, and thus would provide less spatial resolution in nanopore based ionic current measurements. A top-view of MBD bound to dsDNA, derived from the crystal structure of the complex, is shown in FIG. 2B. A cross-sectional diameter of ˜5 nm is estimated for the complex containing a single MBD molecule, significantly larger than the 2.2 nm cross-sectional diameter of B-DNA. With multiple bound MBD proteins, this diameter is estimated at 7.6 nm as methylated binding sites follow the rotation of the major groove on dsDNA (FIG. 9). Gel shift assays (FIG. 2C) are used to optimize binding conditions for complex formation prior to nanopore measurements. In the presence of uDLX1, no complex formation was observed (lanes 1-3). In contrast, when mDLX1 is combined with MBD-1x, robust complex formation is observed (lanes 5-9). Complex formation increases as MBD-1x concentration is increased. Importantly, this protein-DNA complex formation occurs at salt concentrations as high as 600 mM KCl (FIG. 10), which is necessary for achieving high signal to noise ratios in nanopore detection experiments. We estimate that a 30:1 excess of MBD-1x to mDLX1 is sufficient to saturate the available methylated binding sites on the target fragment.

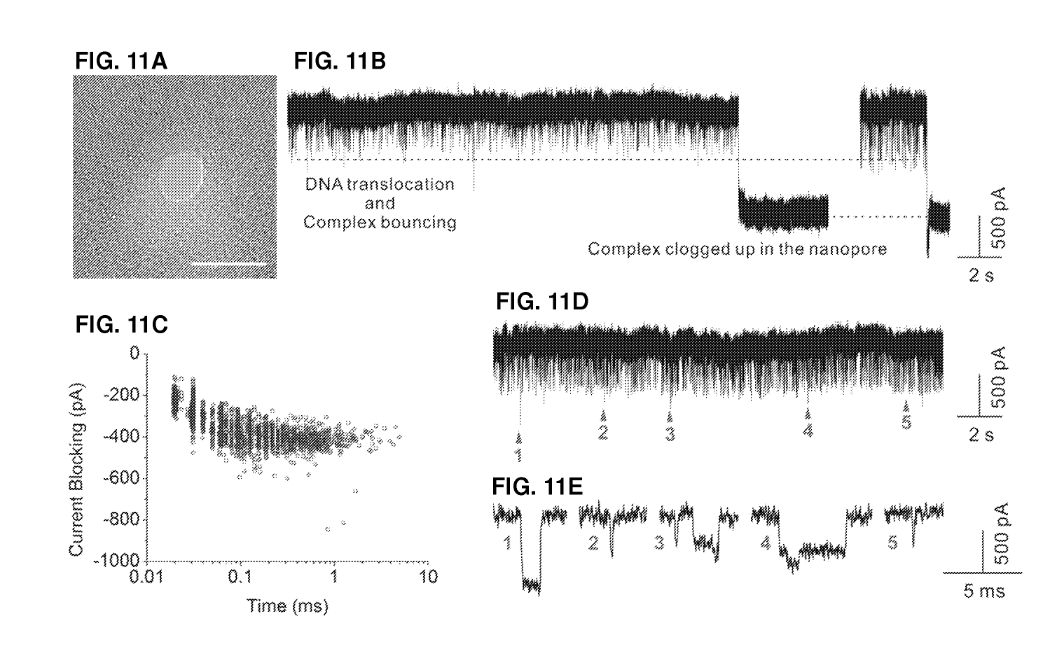

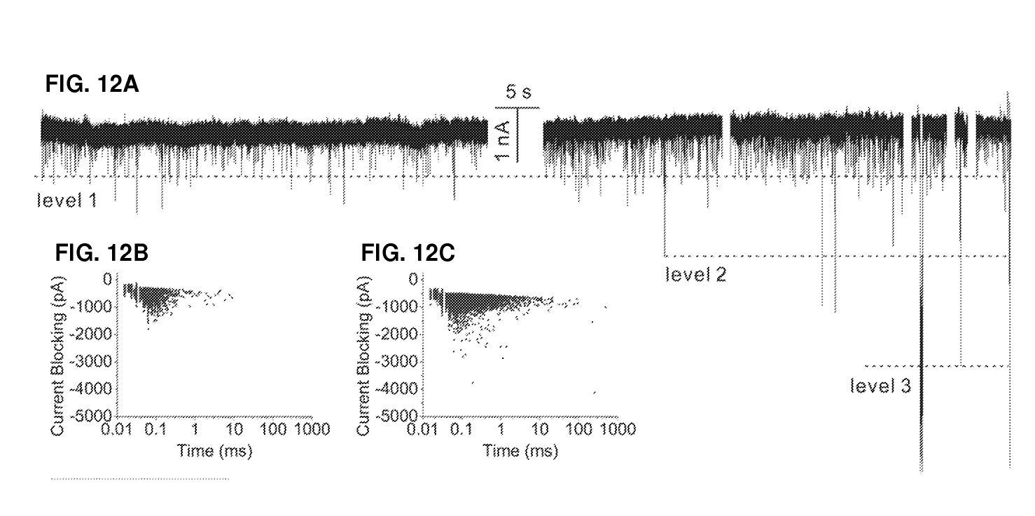

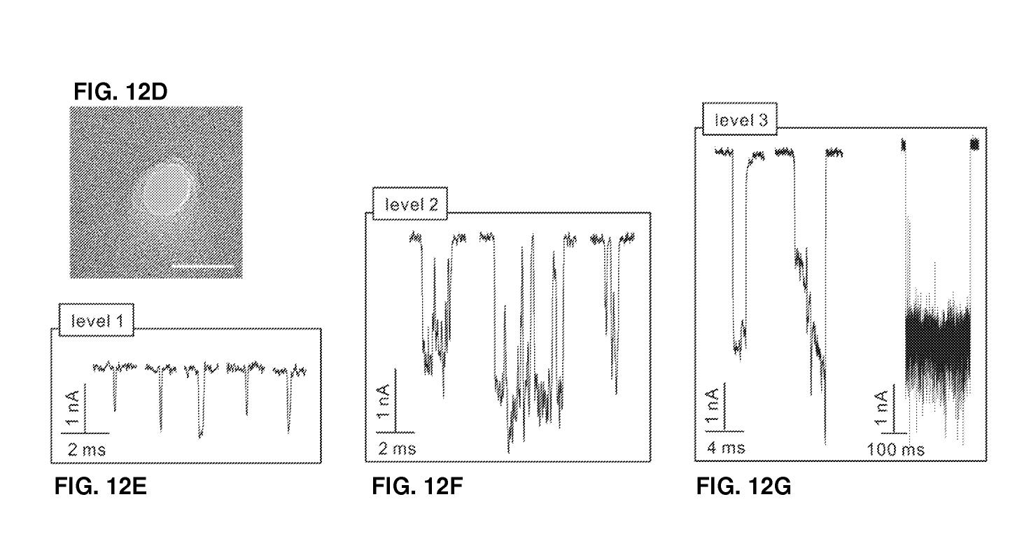

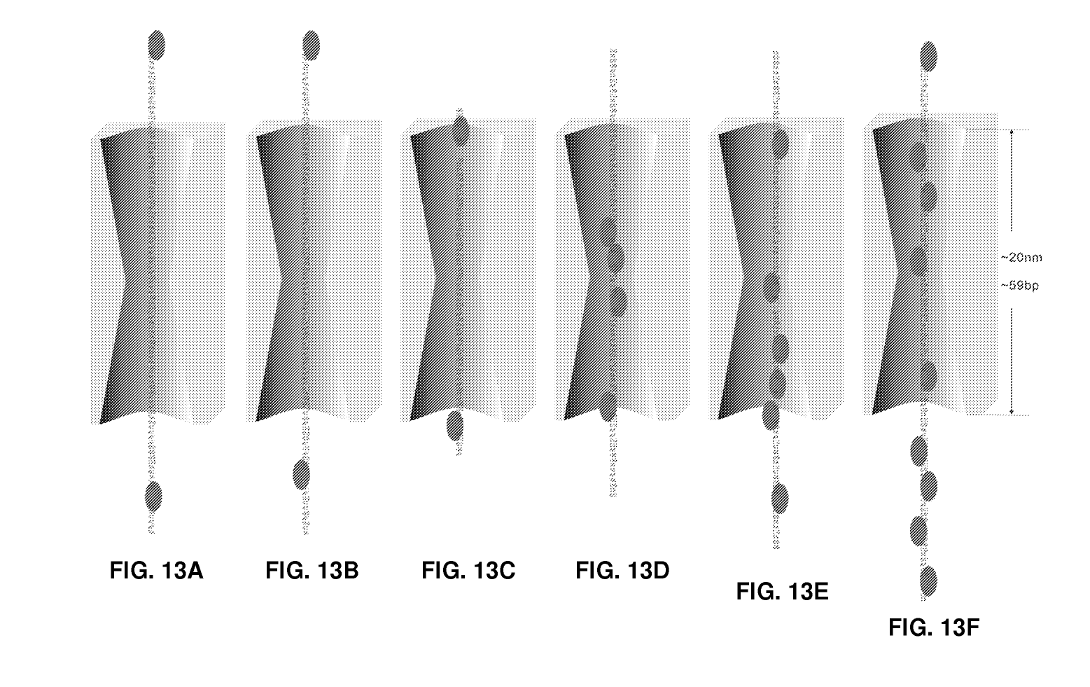

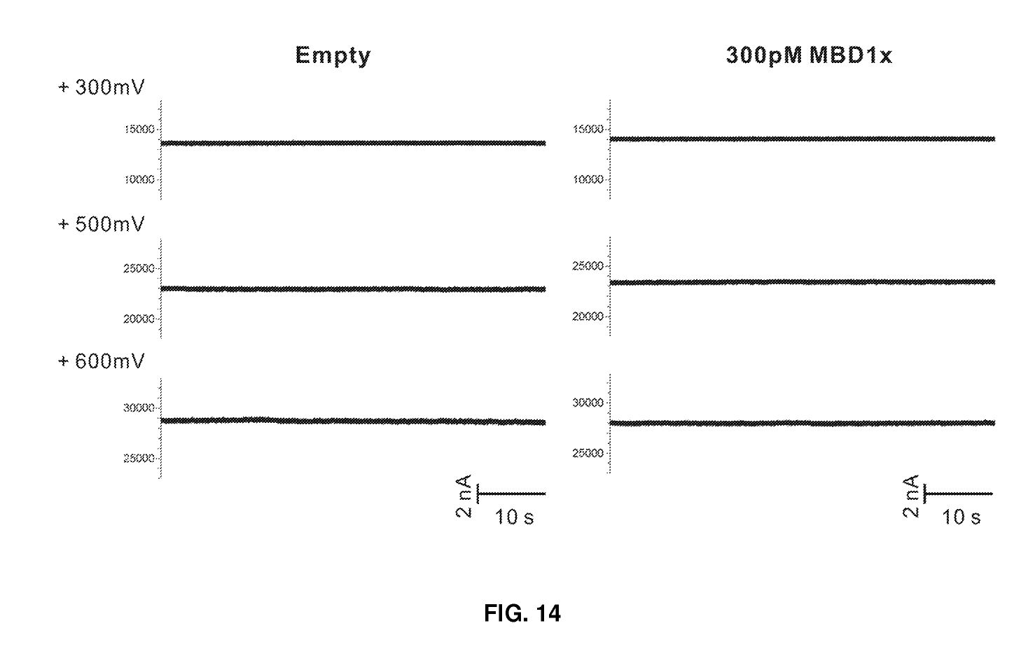

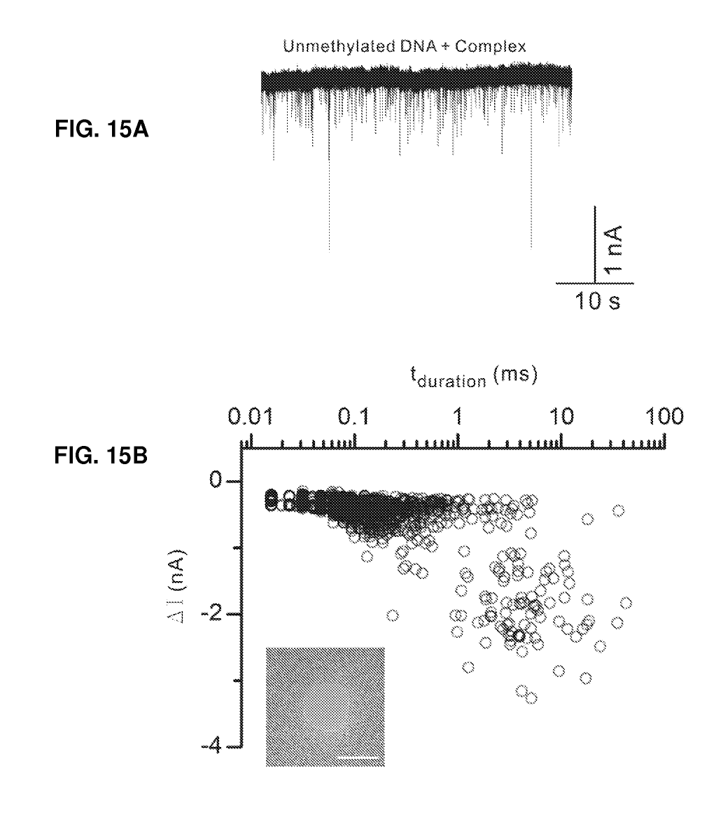

Discrimination of mDLX1/MBD-1x complex from unmethylated DNA: Control experiments with nanopores of diameter of 4.5 nm and 7 nm, where these sizes are comparable with a single MBD-1x bound to DNA (5 nm) and multiple MBD-1x bound to DNA (7.6 nm), show that mDLX1/MBD-1x complex cannot translocate through these pores (FIGS. 11A-11D and 12A-12G). Consequently, we utilize pores with larger diameters than the diameter of mDLX1/MBD-1x complex. The transport of uDLX1 at 1 nM of final concentration and the 1:30 mDLX1/MBD-1x complex at 10 pM through 12 nm diameter pore at an applied voltage of 600 mV is shown in FIG. 2D and characteristic events are shown in FIG. 2E. A lower concentration of mDLX1/MBD-1x complex is used to explore a lower limit of detection. Notably, the transport of the complex induced deeper current blockades and longer translocation times relative to uDLX1. This is best represented in the τ_{d} histogram and ΔI all-points histograms of FIG. 2F consisting of n=857 unmethylated, and n=197 methylated events. Event flux (number of events per second) was expectedly less in the case of the complex versus uDLX1 as the entropic barrier associated with transport of the complex through the pore is significantly higher relative to uDLX1, in addition to more steric hindrance encountered by the complex during translocation. Fitting exponentials to the τ_{d} histogram gave time constants of τ_{M}=1.43±0.03 ms, τ_{U}=0.103±0.005 ms for mDLX1 and uDLX1 respectively, revealing the ability to statistically differentiate these populations. It should be noted that the DNA-protein interactions can be reversible as the K_{D} can be from 106 to 870 nM⁴⁴. This can indeed result in a wider distribution of the translocation duration due to varying number of bound protein on each DNA. However, we also note that the mDLX1/MBD-1x was clearly distinguishable from the uDLX1 since the translocation durations were different by over an order of magnitude. Furthermore, an all-point ΔI histogram provided a detailed view of the translocation of mDLX1/MBD-1x translocation through the nanopore. The ΔI histogram for the mDLX1/MBD-1x complex shows both a deep current blockade level and a shallower blockade level consistent with free DNA in the absence of protein. This demonstrates that the nanopore can indeed coarsely detect protein-bound regions as well as protein-free region on a single molecule, thereby enabling methylation mapping (FIG. 13A-13F). To confirm that the deeper blockade levels observed in the ΔI histogram are due to the DNA/protein complex and not due to the presence of unbound MBD-1x protein, control experiments examining the transport of the free protein are attempted. No free MBD-1x translocation events are observed (FIG. 14), because MBD-1x is positively charged in pH 8 electrolyte, thus will not migrate through the pore under the voltage polarity used in these experiments. Discrimination experiments using a mixture of uDLX1 and the mDLX1/MBD-1x complex are also conducted (FIG. 15A-15B), again with significant differences at deeper current blockage in prolonged translocation were observed in the transport of complex over shallow short duration blockages of uDLX1. These data confirm that a nanopore based technique can differentiate methylated DNA from unmethylated DNA with high confidence using a methylation specific label.

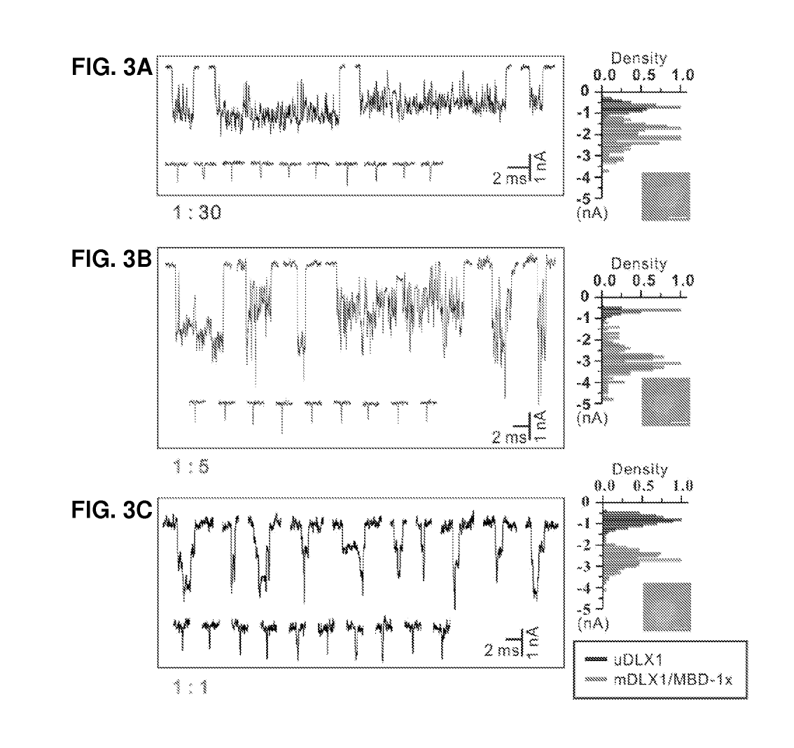

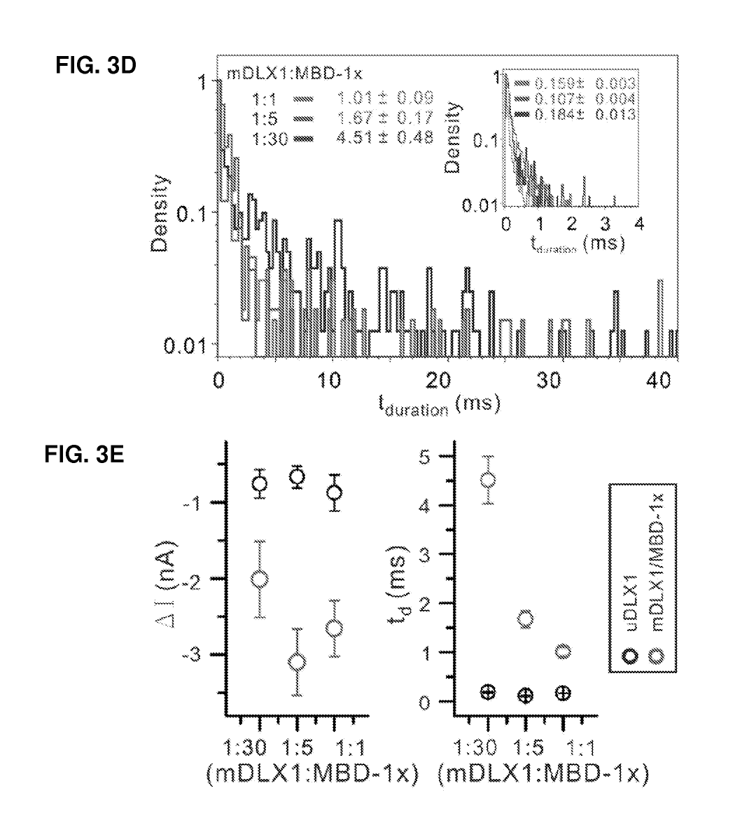

Methylation Quantification: To quantify the extent of DLX1 methylation, various ratios of MBD-1x to mDLX1 are incubated and then translocated through nanopores of diameter ranging from 9 to 10 nm. A pore diameter of 9-10 nm was specifically selected to allow for slower complex translocation. Translocation data for 1:30, 1:5 and 1:1 ratios of mDLX1/MBD-1x are shown in FIGS. 3A-3C, respectively. Each experiment involves translocating uDLX1 as a control fragment (lower insert), followed by translocation of the DNA-protein complex through the same nanopore. Current signatures of uDLX1 and mDLX/MBD-1x complex are compared via histogram of peak blocking current (uDLX1 in black and mDLX1/MBD-1x complex in red) along with a TEM image of each of the nanopore used. FIGS. 3A-3C also qualitatively show that by lowering the ratio of protein to DNA, thereby reducing the mean number of bound proteins per DNA molecule, a measurable reduction in the translocation time of the complex can be observed. This is best visualized in the normalized translocation time histogram in FIG. 3D. As can be seen in FIG. 3E (left panel), for all DNA/protein ratios examined, mDLX1/MBD-1x can be clearly distinguished from uDLX1 based on blockage amplitude, ΔI. The complex remains clearly distinguishable even at the lowest protein/DNA ratios examined. Fitting a Gaussian function to the peak value of the blocking current of ΔI gave current signatures of mDLX1/MBD-1x and uDLX1 at all ratios. Current signatures of mDLX1/MBD-1x complex are obtained at ΔI_{1:30}=−2.01±0.5 nA, ΔI_{1:5}=−3.09±0.44 nA and ΔI_{1:1=}−2.65±0.37 nA, while uDLX1 through the same pores showed current signatures of ΔI_{uDLX1}_{_}_{at1:30}=−0.76±0.19 nA, ΔI_{uDLX1}_{_}_{1:5}=−0.67±0.07 nA and ΔI_{uDLX1}_{_}_{1:1}=−0.87±0.24 nA. Overall, regardless of the ratio of MBD-1x to mDLX1, the nanopore can detect and identify mDLX1/MBD-1x complex from uDLX1 by about a 3-fold larger current signature.

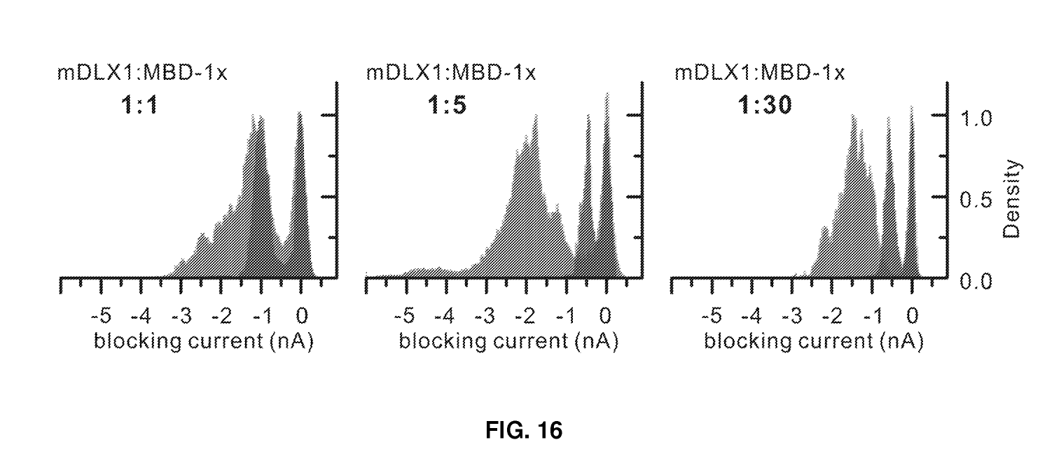

Given a 1:1 DNA-protein ratio, the number of bound proteins per DNA molecule can be calculated using a Poisson limited random statistical distribution⁴⁸. According to this model, the probability that a single DNA molecule will contain one or fewer bound proteins is ˜74%. Therefore, the majority of translocation events observed in FIG. 3C can be credited to the binding of one MBD-1x protein per mDLX1 molecule (free DNA translocation events not included in the histogram), and overlapping all-points histogram of blocking currents between uDLX1 and mDLX1/MBD-1x indicates one or fewer bound protein to the DNA (FIG. 16). Furthermore, as rms current noise is identical in the preceding measurements, we conclude that a methylated DNA fragment with a single bound protein can give a ˜305% enhancement in ionic current relative to unmethylated DNA. This confirms that the nanopore based methylation analysis technique presented here can indeed detect the presence of a single bound protein on average on methylated DNA with the sensitivity of a single CpG dinucleotide.

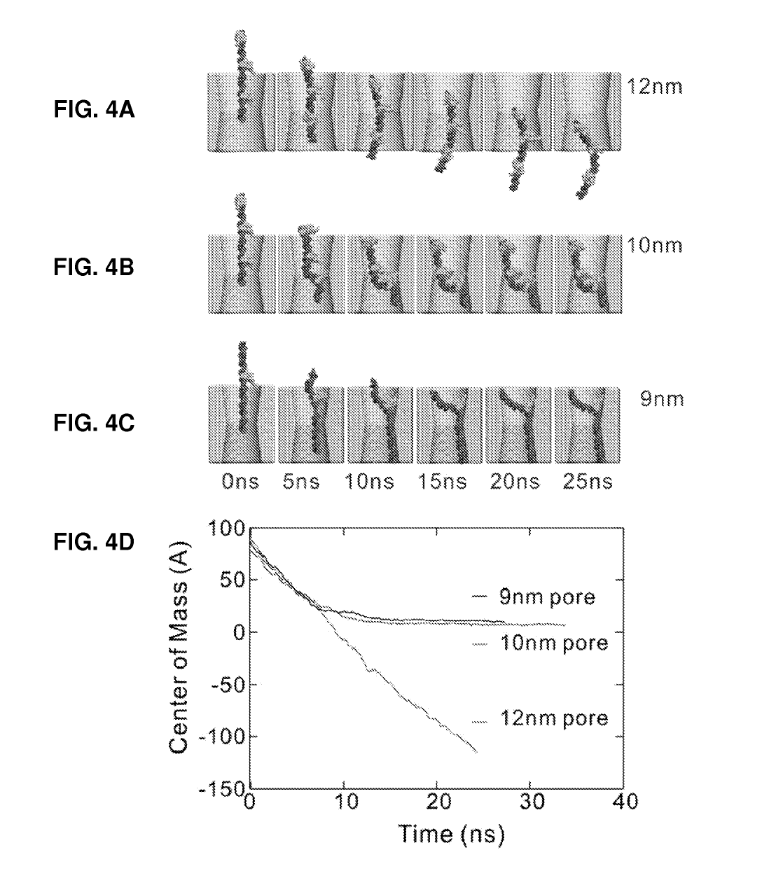

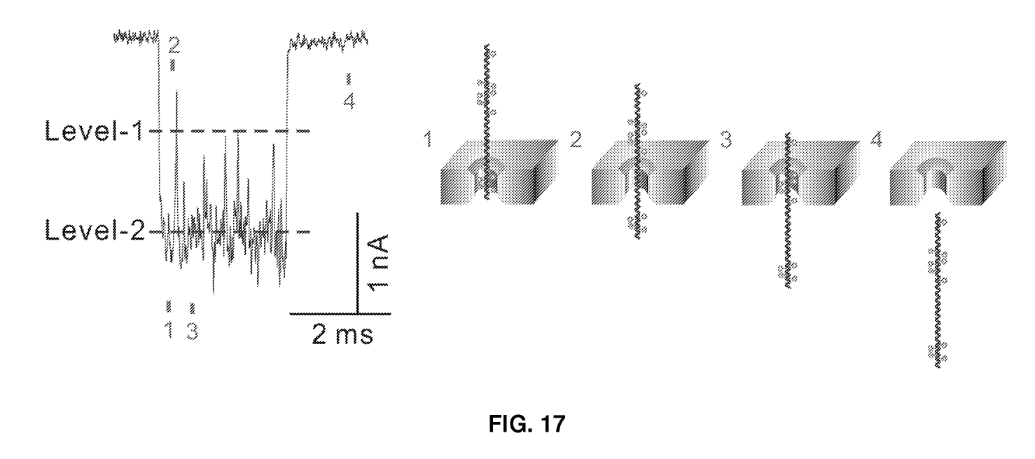

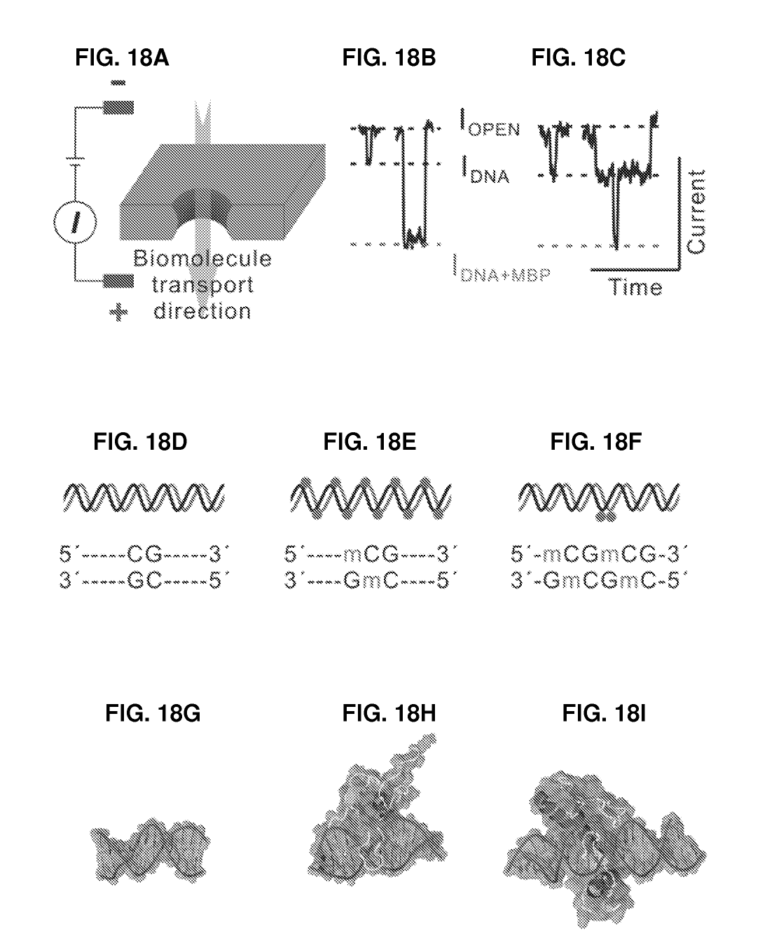

FIG. 3E (right panel), shows distinct time scales of τ_{1:30}=4.51±0.48 ms, τ_{1:5}=1.67±0.17 ms and τ_{1:1}=1.01±0.09 ms calculated for the 1:30, 1:5 and 1:1 distributions respectively, based on an exponential fitting to the histogram in FIG. 3D. Using this method, methylation quantification in the time domain based on the number of bound proteins is indeed possible. The uDLX1 control, fitted to in range of 0.107 0.184 ms, is shown in the inset of FIG. 3D. The distinct time constants pertaining to the complex likely result from translocation involving interactions with the pore walls. To understand the nature of these protein-pore interactions, molecular dynamics (MD) simulations were conducted as shown in FIGS. 4A-4D. FIGS. 4A and 4B illustrate the transport of 63 bp dsDNA with 3 bound MBD proteins through 12 nm and 10 nm diameter nanopores respectively. Temporal snapshots from the MD trajectory reveal that the complex interacts minimally with the pore walls during translocation through a larger 12 nm pore. In contrast, interactions between the complex and the pore are observed in smaller 10 nm pores, the center of mass of the complex remaining anchored in the pore upon completion of the simulation (FIG. 4D). As nanopore diameter is reduced further to 9 nm (FIG. 4C), the presence of even a single protein can induce polymer-pore interactions and the capture of the complex in the pore, resulting in longer blockade times. The simulation results agree with experimental data in general. Time constants for 1:30 mDLX1/MBD-1x complexes through a ˜12 nm pore (1.43±0.03 ms) were more than a factor of 3 less than translocation time constants for 1:30 complexes through a ˜10 nm pore (4.51±0.48 ms), confirming faster translocation through larger pores. Comparable time constants are measured for 1:5 complexes through a 9 nm pore. The detailed view of an experimental data trace from an individual mDLX1/MBD-1x shows slow translocation of the complex due to polymer-pore interactions (FIG. 17). These interactions are both hydrophobic and electrostatic in nature. Once a protein or DNA contacts the pore wall, Van der Waals interactions between the biomolecule and the pore wall slow down the translocation velocity of biomolecule as reported previously with single-stranded DNA⁴⁹. Electrostatic polymer-pore interactions are also likely and have been reported to slow DNA in systems where the nanopore surface charge is opposite in polarity to the charge on the translocating biomolecule^{42,50}. As the experiments were carried out in pH 8 electrolyte and as the isoelectric points of MBD1x and the SiN pore are 8.85 and ˜4 respectively^{51,52}, we expect electrostatic interactions between the positively charged protein and the negatively charged nanopore surface. Thus, longer translocation times are expected as the number of bound proteins per DNA molecule is increased.

This example presents a new solid-state nanopore-based direct electrical analysis technique for detecting unmethylated and methylated DNA at the single molecule level. Using MBD-1x as a methylation specific label, the methylation status of nucleotide sequences corresponding to the promoter of DLX1, a potential epigenetic biomarker for cancer, could be rapidly determined without the need for bisulfite conversion, sequencing or fluorescent tags. Notably, the translocation of the mDLX1-protein complex versus uDLX1 induces an about 3-fold signal enhancement in the pore blockage current, enabling the electrical detection of a single methylated CpG dinucleotide-protein complex with high fidelity. The number of methylation sites per molecule can also be coarsely determined using this approach based on the number of bound MDB-1x proteins, characterized by distinct timescales in the corresponding translocation time histograms. Additional studies will determine the ultimate spatial resolution of this technique, these findings have an application in low-resolution gene based methylation analysis and the mapping of methylated CpG islands in the promoter sequences of various genes, essential to transcriptional repression and gene silencing³. Extending this technique to high resolution epigenetic mapping requires further improvements to the nanopore architecture. Nanopores used in these studies are 20 nm-thick in length (equivalent to ˜60 bps of dsDNA) and thus multiple bound proteins contributed to the measured ionic current (FIG. 13). By reducing pore thickness to below the size of an individual protein, for example by using monolayer thick graphene nanopores⁵³ (thickness of ˜0.34 nm), it may be possible to accurately quantify and spatially map the location of individual MBD-1x proteins on a target DNA molecule. This should be feasible as the translocation of DNA-protein complexes through graphene nanopores has already been demonstrated⁵⁴. Such a technology has application in clinical settings. Cancer-specific methylated DNA from most tumor types are known to be present in biopsy specimens and in patient serum at very low concentrations. A rapid, accurate and amplification free assay to detect these biomarkers from minute sample volumes could prove invaluable in the early detection of disease, monitoring disease progression and prognosis. Solid-state nanopores can meet this unmet technological and clinical need.

Methods. Nanopore electrical measurements: Single nanopores of various diameters are sculpted using a JEOL 2010F field emission gun transmission electron microscope in 20 nm thick, low stress SiN membranes with window sizes of 50×50 μm², supported on a silicon chip. Following pore formation, nanopore chips were cleaned in Piranha solution (two parts 95% H₂SO₄ and one part of 30% of H₂O₂) for 10 min and thoroughly rinsed with DI H₂O. The chip was then sandwiched in a custom acrylic holder with the nanopore forming the only electrical path for ions between the two reservoirs. The recording solution for both sides was prepared with desired concentrations of KCl at pH 8.0 with 10 mM Tris.HCl and 1 mM EDTA. Ag/AgCl electrodes were immersed in the two reservoirs and an Axopatch 200B was used for applying potentials and measuring currents at a bandwidth of 10 kHz. Data was recorded at a sampling frequency of 100 kHz using a Digidata 1440A data acquisition system. Instrumental control and data analysis was performed using Clampex 10.2. All nanopore experiments were performed in a dark, double Faraday cage on an anti-vibration table at room temperature (22±2° 0).

DNA Preparation, Purification and Methylation: The 827 bp DNA fragment used was generated by conventional PCR of human genomic DNA (G304A, Promega, Madison, Wis.) and includes a region of the DLX1 gene (Homo sapiens distal-less homeobox). The region includes a nontranscribed area adjacent to a CpG island, the 5′ untranslated region (UTR), the complete first exon, part of the first intron and 36 potential CpG sites. The PCR primer sequences are; forward: gaccaatccccagtgattatgcaagac, reverse: ctcaatttgcaactatccagccaagg (as illustrated in FIG. 5). The PCR product was purified using Qiaquick PCR purification kit (Qiagen, Inc., Valencia, Calif.). 50 μg of DNA was methylated in 10 ml using 500 U CpG Methyltransferase M.Sssl, New England Biolabs (Ipswich, Mass.) #M0226M, and 160 μM s-adenosyl-methionine (SAM) according to manufacturer's instructions. 33 μg of unmethylated control DNA was treated in the same manner except that no M.Sssl was included in the reaction. Reactions were carried out at 37° C. for 4 hours, then fresh SAM was added again to 160 μM (320 μM total) and incubated for another 4 hours. DNA was precipitated with ethanol and agarose gel purification was performed using Qiaquick kit with gel extraction protocol. Efficiency of methylation was shown to be high by nearly complete protection from Hhal (a methylation sensitive enzyme) restriction digestion. There are 4 Hhal restriction sites in this 827 bp fragment.

MBD-1x protein purification: BL21 DE3pLysS E. coli that had been transformed with a bacterial expression vector encoding his-tagged MBD-1x was exposed to 1 mM IPTG and incubated on an orbital shaker at 37° C. for 3 hours. Bacteria was then chilled on ice, centrifuged at 5000×g for 5 minutes at 4° C. and subjected to 3 freeze/thaw cycles. Lysis buffer (50 mM NaH₂PO₄, 300 mM NaCl, 10 mM imidazole, pH 8.0) was added and the bacterial lysate was sonicated and spun at 10,000×g for 40 minutes at 4° C. The cleared lysate was added to a column packed with nickel-NTA agarose resin (Quiagen, Valencia, Calif.) on an Econo Protein Purification System (BioRad, Hercules, Calif.) and incubated for 2 hours to allow the his-tagged protein to bind to the nickel column. Guanadinium hydrochloride (5.5 M) was added to the column to denature the protein and a linear guanadinium hydrochloride gradient (5.5-0 M) was used to refold the protein. This renaturation step was critical for MBD-1x activity. The refolded MBD-1x was eluted with increasing concentrations of imidazole (10-250 mM) in elution buffer (50 mM NaH₂PO₄, 300 mM NaCl, pH 8.0). Protein purity was assessed with Coomassie-stained gels and Western blot analysis using an anti-His antibody (SC-803, Santa Cruz Biotechnology, Santa Cruz, Calif.).

Gel shift assays: The 827 bp uDLX1 and mDLX1 DNA was end labeled with y[³¹P]ATP and T4 polynucleotide kinase (New England Biolabs, Ipswich, Mass.) and the radiolabeled DNA was separated from free ³¹P using Quick Spin Columns (Roche Diagnostics Corporation, Indianapolis, Ind.). The indicated amounts of purified MBD-1x were added to binding buffer (15 mM Tris pH 7.5, 80 mM KCl, 0.4 mM dithiothreitol, 0.2 mM EDTA, 1 ug poly[deoxyinosine/deoxycytosine], 10% glycerol) and incubated for 15 minutes at room temperature. Radiolabeled DNA was added and incubated for 25 minutes at room temperature in a final volume of 20 μl. Samples were fractionated on a low-ionic strength polyacrylamide gel at 4° C. with buffer recirculation as previously described⁵⁵. Bands were visualized using autoradiography.

Molecular dynamics simulation—atomic model: The atomic model of silicon nitride membrane was constructed as described previously⁴⁹. The thickness of the membrane is 20 nm. A symmetric double-conical pore was produced by removing atoms from the silicon nitride membrane with the diameter of the pore corresponds to experiment (9 nm, 10 nm and 12 nm). Atomic coordinates of mDNA-MBD complex were taken from the NMR structure of the methyl binding domain of MBD1 complexed with mDNA (Protein Data Bank entry code 1 IG4⁴⁶). Three mDNA-MBD complex were linked together to generate a long mDNA binding with three MBD proteins, see FIG. 4A-4D. The sequence of DNA is: 5′-TATCmCGGATACGTATCCGGTATCmCGGATACGTATC CGGATATATCmCGGATACGTATCCGGATA-3′. The specific binding sites (mCG) of mDNA are marked in red. The topology file of DNA and protein along with the missing hydrogen atoms was generated using the psfgen plug-in of VMD⁵⁶. mDNA-MBD complex was placed in front of the pore and was solvated in a water box with 0.6 M KCl added. The final systems include ˜1.1 million atoms. Simulations were performed using the program NAMD 2.8 with the CHARMM27 force field for DNA⁵⁷, the CHARMM22 force field for proteins with CMAP corrections^{58,59} and the TIP3P water model⁶⁰. Periodic boundary condition was employed. The integration time step used was 1 fs with particle-mesh Ewald (PME) full electrostatics with grid density of 1/Å³. Van der Waals energies were calculated using a 12 Å cutoff. A Langevin thermostat was assumed to maintain constant temperature at 295 K⁶¹. Each system was energy-minimized for 30,000 steps and then equilibrated for 2 ns under NPT ensemble condition to achieve a constant volume^{61,62}. Production simulations were carried out by applying an electric field along the z-direction (perpendicular to the membrane). The applied voltage is 0.6 V as employed in experiments. Shim, J. et al. Sci. Rep. 3:1389 (Mar. 11, 2013).

### REFERENCES FOR EXAMPLE 1

1. Robertson, K. D. DNA methylation and human disease. Nat Rev Genet 6, 597-610 (2005).
2. Rountree, M. R., Bachman, K. E., Herman, J. G. & Baylin, S. B. DNA methylation, chromatin inheritance, and cancer. Oncogene 20, 3156-3165 (2001).
3. Das, P. M. & Singal, R. DNA methylation and cancer. J. Clin. Oncol. 22, 4632-4642 (2004).
4. Laird, P. W. The power and the promise of DNA methylation markers. Nat. Rev. Cancer 3, 253-266 (2003).
5. Laird, P. W. & Jaenisch, R. The role of DNA methylation in cancer genetics and epigenetics. Annu. Rev. Genet. 30, 441-464 (1996).
6. Strathdee, G. & Brown, R. Aberrant DNA methylation in cancer: potential clinical interventions. Expert Reviews In Molecular Medicine 4, 1-17 (2002).
7. Vanaja, D. K. et al. Hypermethylation of genes for diagnosis and risk stratification of prostate cancer. Cancer Invest. 27, 549-560 (2009).
8. Esteller, M., Corn, P. G., Baylin, S. B. & Herman, J. G. A gene hypermethylation profile of human cancer. Cancer Res. 61, 3225-3229 (2001).
9. Lee, W. H., Isaacs, W. B., Bova, G. S. & Nelson, W. G. CG island methylation changes near the GSTP1 gene in prostatic carcinoma cells detected using the polymerase chain reaction: A new prostate cancer biomarker. Cancer Epidemiology Biomarkers And Prevention 6, 443-450 (1997).
10. Murrell, A., Rakyan, V. K. & Beck, S. From genome to epigenome. Hum. Mol. Genet. 14, R3-10 (2005).
11. Grunau, C., Clark, S. J. & Rosenthal, A. Bisulfite genomic sequencing: systematic investigation of critical experimental parameters. Nucleic Acids Res. 29, e65 (2001).
12. Trinh, B. N., Long, T. I. & Laird, P. W. DNA methylation analysis by methyLight technology. Methods 25, 456-462 (2001).
13. Eads, C. A. et al. MethyLight: a high-throughput assay to measure DNA methylation. Nucleic Acids Res. 28, e32 (2000).
14. Chargari, C., Campana, F., Beuzeboc, P., Zefkili, S. & Kirova, Y. M. Preliminary experience of helical tomotherapy for locally advanced pancreatic cancer. World Journal Of Gastroenterology: WJG 15, 4444-4445 (2009).
15. Sandoval, J. et al. Validation of a DNA methylation microarray for 450,000 CpG sites in the human genome. Epigenetics 6, 692-702 (2011).
16. Venkatesan, B. M. & Bashir, R. Nanopore sensors for nucleic acid analysis. Nat Nano 6, 615-624 (2011).
17. Kasianowicz, J. J., Brandin, E., Branton, D. & Deamer, D. W. Characterization of individual polynucleotide molecules using a membrane channel. Proceedings Of The National Academy Of Sciences Of The United States Of America 93, 13770-13773 (1996).
18. Kim, M. J., Wanunu, M. & Bell, D. C. A. Meller Rapid fabrication of uniformly sized nanopores and nanopore arrays for parallel DNA analysis. Advanced Materials 18, 3149-3153 (2006).
19. Venkatesan, B. M. et al. Highly sensitive, mechanically stable nanopore sensors for DNA analysis. Advanced Materials 21, 2771-2776 (2009).
20. Dekker, C. Solid-state nanopores. Nature Nanotechnology 2, 209-215 (2007).
21. Branton, D. et al. The potential and challenges of nanopore sequencing. Nature Biotechnology 26, 1146 (2008).
22. Kasianowicz, J. J., Robertson, J. W. F., Chan, E. R., Reiner, J. E. & Stanford, V. M. Nanoscopic porous sensors. Annu Rev Anal Chem 1, 737-766 (2008).
23. Reiner, J. E. et al. Disease detection and management via single nanopore-based sensors. Chem Rev 112, 6431-6451 (2012). 24. Bayley, H. & Jayasinghe, L. Functional engineered channels and pores—(Review). Mol Membr Biol 21, 209-220 (2004).
25. Gu, L. Q. & Shim, J. W. Single molecule sensing by nanopores and nanopore devices. Analyst 135, 441-451 (2010).
26. Howorka, S. & Siwy, Z. Nanopore analytics: sensing of single molecules. Chem Soc Rev 38, 2360-2384 (2009).
27. Ma, L. & Cockroft, S. L. Biological nanopores for single-molecule biophysics. Chembiochem 11, 25-34 (2010).
28. Movileanu, L. Interrogating single proteins through nanopores: challenges and opportunities. Trends Biotechnol 27, 333-341 (2009).
29. Branton, D. et al. The potential and challenges of nanopore sequencing. Nature Biotechnology 26, 1146-1153 (2008).
30. Olasagasti, F. et al. Replication of individual DNA molecules under electronic control using a protein nanopore. Nat Nanotechnol 5, 798-806 (2010).
31. Cipriany, B. R. et al. Single molecule epigenetic analysis in a nanofluidic channel. Anal Chem 82, 2480-2487 (2010).
32. Cipriany, B. R. et al. Real-time analysis and selection of methylated DNA by fluorescence-activated single molecule sorting in a nanofluidic channel. Proceedings Of The National Academy Of Sciences Of The United States Of America 109, 8477-8482 (2012).
33. Clarke, J. et al. Continuous base identification for single-molecule nanopore DNA sequencing. Nat Nanotechnol 4, 265-270 (2009).
34. Mirsaidov, U. et al. Nanoelectromechanics of methylated DNA in a synthetic nanopore. Biophysical Journal 96, L32-L34 (2009).
35. Wanunu, M. et al. Discrimination of methylcytosine from hydroxymethylcytosine in DNA molecules. J. Am. Chem. Soc. 133, 486-492 (2010).
36. Bird, A. DNA methylation patterns and epigenetic memory. Genes Dev. 16, 6-21 (2002).
37. Petryniak, M. A., Potter, G. B., Rowitch, D. H. & Rubenstein, J. L. Dlx1 and Dlx2 control neuronal versus oligodendroglial cell fate acquisition in the developing forebrain. Neuron 55, 417-433 (2007).
38. Tong, W. G. et al. Genome-wide DNA methylation profiling of chronic lymphocytic leukemia allows identification of epigenetically repressed molecular pathways with clinical impact. Epigenetics: Official Journal Of The DNA Methylation Society 5, 499-508 (2010).
39. Wu, X. et al. CpG island hypermethylation in human astrocytomas. Cancer Res 70, 2718-2727 (2010).
40. Selamat, S. A. et al. Genome-scale analysis of DNA methylation in lung adenocarcinoma and integration with mRNA expression. Genome Res 22, 1197-1211 (2012).
41. Wanunu, M., Sutin, J., McNally, B., Chow, A. & Meller, A. DNA translocation governed by interactions with solid-state nanopores. Biophys J 95, 4716-4725 (2008).
42. Venkatesan, B. M., Shah, A. B., Zuo, J. M. & Bashir, R. DNA sensing using nanocrystalline surface-enhanced Al2O3 nanopore sensors. Adv Funct Mater 20, 1266-1275 (2010).
43. Roloff, T., Ropers, H. H. & Nuber, U. Comparative study of methyl-CpG-binding domain proteins. BMC Genomics 4, 1 (2003).
44. Yu, Y. N. et al. Direct DNA methylation profiling using methyl binding domain proteins. Anal Chem 82, 5012-5019 (2010).
45. Drew, H. R. et al. Structure of a B-DNA dodecamer—conformation and dynamics. 1. P Natl Acad Sci-Biol 78, 2179-2183 (1981).
46. Ohki, I. et al. Solution structure of the methyl-CpG binding domain of human MBD1 in complex with methylated DNA. Cell 105, 487-497 (2001).
47. Nan, X., Meehan, R. R. & Bird, A. Dissection of the methyl-CpG binding domain from the chromosomal protein MeCP2. Nucleic Acids Res. 21, 4886-4892 (1993).
48. Eid J., et al. (ed. USPTO) (Pacific Biosciences of California, Inc., USA; 2010).
49. Aksimentiev, A., Heng, J. B., Timp, G. & Schulten, K. Microscopic kinetics of DNA translocation through synthetic nanopores. Biophysical Journal 87, 2086-2097 (2004).
50. Rincon-Restrepo, M., Mikhailova, E., Bayley, H. & Maglia, G. Controlled translocation of individual DNA molecules through protein nanopores with engineered molecular brakes. Nano Letters 11, 746-750 (2011).
51. Jorgensen, H. F., Adie, K., Chaubert, P. & Bird, A. P. Engineering a high-affinity methyl-CpG-binding protein. Nucleic Acids Res 34 (2006).
52. Hoogerheide, D. P., Garaj, S. & Golovchenko, J. A. Probing surface charge fluctuations with solid-State nanopores. Phys. Rev. Lett. 102, 256804 (2009).
53. Garaj, S. et al. Graphene as a subnanometre trans-electrode membrane. Nature 467, 190-193 (2010).
54. Venkatesan, B. M. et al. Stacked graphene-Al2O3 nanopore sensors for sensitive detection of DNA and DNA-protein complexes. ACS Nano 6, 441-450 (2011).
55. Loven, M. A., Wood, J. R. & Nardulli, A. M. Interaction of estrogen receptors alpha and beta with estrogen response elements. Mol Cell Endocrinol 181, 151-163 (2001).
56. Humphrey, W., Dalke, A. & Schulten, K. VMD: Visual molecular dynamics. J Mol Graph Model 4, 33-38 (1996).
57. Foloppe, N. & MacKerell, A. D. All-atom empirical force field for nucleic acids: I. Parameter optimization based on small molecule and condensed phase macromolecular target data. J Comput Chem 21, 86-104 (2000).
58. MacKerell, A. D. et al. All-atom empirical potential for molecular modeling and dynamics studies of proteins. J Phys Chem B 102, 3586-3616 (1998).
59. Mackerell, A. D., Feig, M. & Brooks, C. L. Extending the treatment of backbone energetics in protein force fields: Limitations of gas-phase quantum mechanics in reproducing protein conformational distributions in molecular dynamics simulations. J Comput Chem 25, 1400-1415 (2004).
60. Jorgensen, W. L., Chandrasekhar, J., Madura, J. D., Impey, R. W. & Klein, M. L. Comparison of simple potential functions for simulating liquid water. J Chem Phys 79, 926-935 (1983).
61. Martyna, G. J., Tobias, D. J. & Klein, M. L. Constant-pressure molecular-dynamics algorithms. J Chem Phys 101, 4177-4189 (1994).
62. Feller, S. E., Zhang, Y. H., Pastor, R. W. & Brooks, B. R. Constant-pressure molecular-dynamics simulation—the langevin piston method. J Chem Phys 103, 4613-4621 (1995).

### Example 2

### Nanopore-Based Assay for Detection of Methylation in Double-Strand DNA Fragments

DNA methylation is an epigenetic modification of DNA in which methyl groups are added at the 5-carbon position of cytosine. Aberrant DNA methylation, which has been associated with carcinogenesis, can be assessed in various biological fluids and potentially can be used as markers for detection of cancer. Analytically sensitive and specific assays for methylation targeting low-abundance and fragmented DNA are needed for optimal clinical diagnosis and prognosis. We present a nanopore-based direct methylation detection assay that circumvents bisulfite conversion and polymerase chain reaction amplification. Building on Example 1, we use methyl-binding proteins (MBPs), which selectively label the methylated DNA. The nanopore-based assay selectively detects methylated DNA/MBP complexes through a 19 nm nanopore with significantly deeper and prolonged nanopore ionic current blocking, while unmethylated DNA molecules were not detectable due to their smaller diameter. Discrimination of hypermethylated and unmethylated DNA on 90, 60, and 30 bp DNA fragments was demonstrated using sub-10 nm nanopores. Hypermethylated DNA fragments fully bound with MBPs are differentiated from unmethylated DNA at 2.1- to 6.5-fold current blockades and 4.5- to 23.3-fold transport durations. Furthermore, these nanopore assays can detect the CpG dyad in DNA fragments and can be used to profile the position of methylated CpG sites on DNA fragments.

Epigenetic alterations involving DNA methylation, which include addition and/or removal of a methyl group at the 5-position of cytosine, are early and frequently observed events in carcinogenesis.¹³ Aberrant methylation occurs in the promoter sequences of various genes linked to many tumors.₄_{_}₆ Hypermethylation is reported to be associated with cancers of the prostate, colon, lung, liver, breast, head and neck and further correlated with metastatic potential in many other tumor types.^{4,6-10} Also, high-throughput methylation analysis has uncovered aberrant DNA methylation in both premalignant and malignant neoplasia.¹¹⁻¹⁴ Hypomethylation is reported to be associated with cancers of the kidney, stomach, liver, colon, pancreas, uterus, cervix, and lung.^{12,15-22} Thus, methylation analysis in DNA can play a critical role in the diagnosis of cancer, especially at an early, precancerous stage.

Previous studies have demonstrated the feasibility of detecting cancer by assessing methylation patterns from genomic extracts of body fluids such as plasma, serum, urine, and stool.^{4,23-25} However, the level of methylated DNA in these fluids is extremely low,²⁶ and the size of the DNA fragments is quite small.²⁷ As a result, most conventional methylation assays require large sample volumes. In addition to the DNA fragmentation that occurs in vivo, bisulfite conversion can lead to further DNA degradation,^{28,29} which additionally compromises the detection sensitivity of conventional methylation detection assays. Finally, most current assays employ polymerase chain reaction (PCR) amplification, which can introduce false-positive results.⁴ Thus, a simple, rapid, and reliable method to detect epigenetic modification of DNA, which uses small samples and eliminates bisulfite treatment and PCR amplification, has potential to revolutionize cancer diagnostics.

Here, we demonstrate a novel strategy to detect varying levels of methylation levels on double-stranded (ds) DNA using a solid-state nanopore-based sensor. The nanopore has been adapted to explore many biophysical questions through single-molecule investigation^{8,30-38} and in applications toward next generation DNA sequencing.^{39,40} A single-molecule detection technology using a nanopore sensor could be well-suited for gene-based methylation analysis,^{33,41} and here we demonstrate the capacity of nanopore sensors to detect methylation in 30, 60, and 90 bp double-stranded oligos. This approach is compatible with small amounts of genomic extracts and direct methylation detection without fluorescence labeling and bisulfite conversion. When integrated with sample preparation, the use of nanopore-based discrimination of various methylation patterns can provide a simple and affordable approach to early cancer detection.

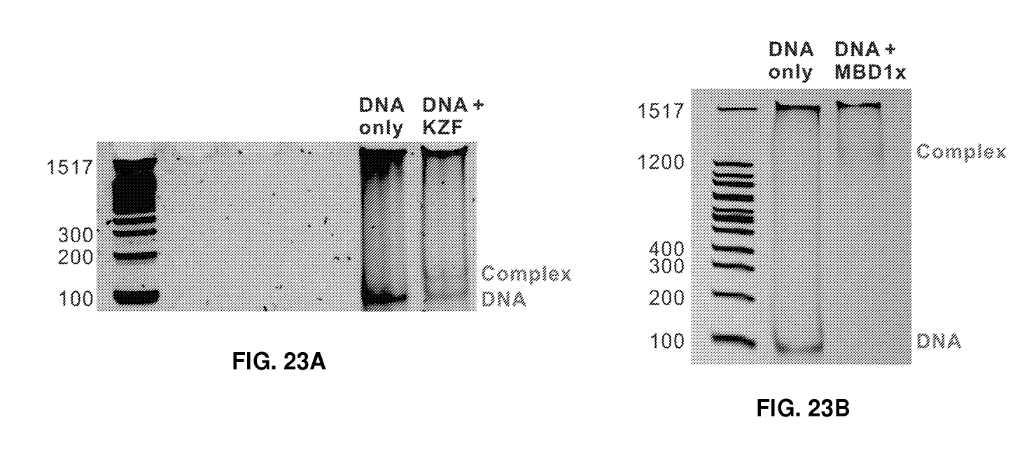

Discrimination of a Variety of Methylation Levels in DNA Fragments: Nanopore-based sensors can detect single molecules as they traverse through a nanopore and alter the background ionic current. Using the principle of electrical current spectroscopy to interrogate biomolecules at the single-molecule level, the sensors can discern subtle structural motifs through sensitive detection of electrical current signatures. The crosssectional view of a solid-state nanopore is illustrated in FIG. 18A. A focused electron beam is used to drill a nanopore within a thin dielectric membrane such as SiN, Al₂O₃, or HfO₂O₂.^{33,42,43} Two reservoir chambers clamp the nanopore membrane from both sides to create a giga-Ohm seal between the two chambers, making the nanopore the only single path of ionic current. The two reservoir chambers contain an electrolyte solution, and the charged single molecules are transported through the nanopore when a bias voltage is applied across the two chambers. Discrimination of the methylation state of 90 bp dsDNA oligos was first demonstrated at the single-molecule level using solid-state nanopores. FIGS. 18B-18C show representative single-molecule transport events. Two distinct nanopore current signatures are observed; the shallow events correspond to transport of naked DNA (left events in FIGS. 18B-18C), and the deeper events correspond to the transport of bound protein (right events in FIGS. 18B-18C). The target dsDNA utilized comprised unmethylated dsDNA (unMethDNA, FIG. 18D), hypermethylated dsDNA (hyMethDNA) with 10 methylated CpGs uniformly distributed through the DNA sequence (FIG. 18E), or locally methylated dsDNA (loMethDNA) with two repetitive methylated CpGs at the center of the sequence (FIG. 18F). The DNA sequence of the unMethDNA is identical to that of the hyMethDNA and loMethDNA sequences but contained no methylated sites. DNA sequenced used herein are described in FIG. 48. Methylation sites in DNA fragments are labeled with methyl-binding proteins (MBPs). Two types of MBPs were used for labeling: MBD1x and KZF. Electrophoretic mobility shift assays for methylated DNA and MBP interactions are shown in FIG. 23A-23B. These MBPs recognize and bind specifically to methylated CpGs; MBD1x is the key methyl-CpG-binding domain of methyl-CpGbinding domain protein (MBD),⁴⁴ and KZF is the key methylation binding domain of Kaiso zinc finger (KZF) protein.⁴⁵ Kaiso is a Cys2_His2 zinc finger protein that binds to methylated CpG and a sequence-specific DNA target. The sequence of KZF contains all three fingers (aa472_573).⁴⁵ The sequence excludes the extra C-terminal domain to prevent nonspecific binding on DNA because some C2H2 KZF utilizes extra domains for nonspecific binding on DNA.⁴⁶ In a similar fashion, Kaiso contains an arginine/lysine-rich region on its C-terminal end, which forms structured loops upon DNA binding that stabilize the contact but also increase nonspecific target binding. The small dimensions of these MBPs contribute to making nanopore-based detection feasible for naked DNA. MBD1x spans 5-6 bps on DNA upon binding and has a molecular weight of 16.3 kDa,⁴⁴ and KZF wraps around 5-6 bps of DNA and has a molecular weight of 13.02 kDa.⁴⁵ The crystal structure of typical B-form DNA⁴⁷ is shown in FIG. 18G, and the two MBPs on methylated DNA are shown in FIG. 18h for MBD1x⁴⁸ and FIG. 18I for KZF.⁴⁵ The MBPs were incubated with methylated DNA at room temperature for 15 min to form the methylated DNA/MBP complex prior to the nanopore-based methylation assay. The passage of the MBP-bound methylated DNA through the nanopore resulted in a significantly different current signature compared to the passage of naked DNA. Because the pore current blockade depends on the cross-sectional diameter of the translocating molecule, a deeper current blockade should be observed when the protein-bound DNA traverses the nanopore (shown in FIGS. 18B-18C). Nanopore-based single-molecule detection through sub-10 nm nanopores identified different methylation profiles (shown in FIG. 18D-18F for unmethylated, hypermethylated, and locally methylated, respectively) on the dsDNA fragment with significantly different electrical current signatures. The hyMethDNA/MBD1x complex could be distinguished from unMethDNA by various passage parameter outputs, including the prolonged translocation time (Δt) and increased current blocking (FIG. 18B). The loMethDNA/KZF complex transport also produced prolonged Δt and stepwise current blocking (FIG. 18C). The extended transport duration of the DNA/complexes was attributed to the net positive charge of MBD1x and KZF in the pH 7.6 nanopore assay buffer solution, which helped to reduce the velocity of complex transport through the negatively charged SiN nanopore.

The methylated DNA detection method provided herein does not require bisulfite conversion and PCR amplification as is required for conventional methylation detection²⁹ or fluorescent tags that are required for optical analysis.⁴⁹ Rather, this nanopore-based detection method relies on direct, single-molecule electrical detection. Consequently, nanopore-based methylated DNA detection are useful in rapid screening for epigenetic biomarkers.

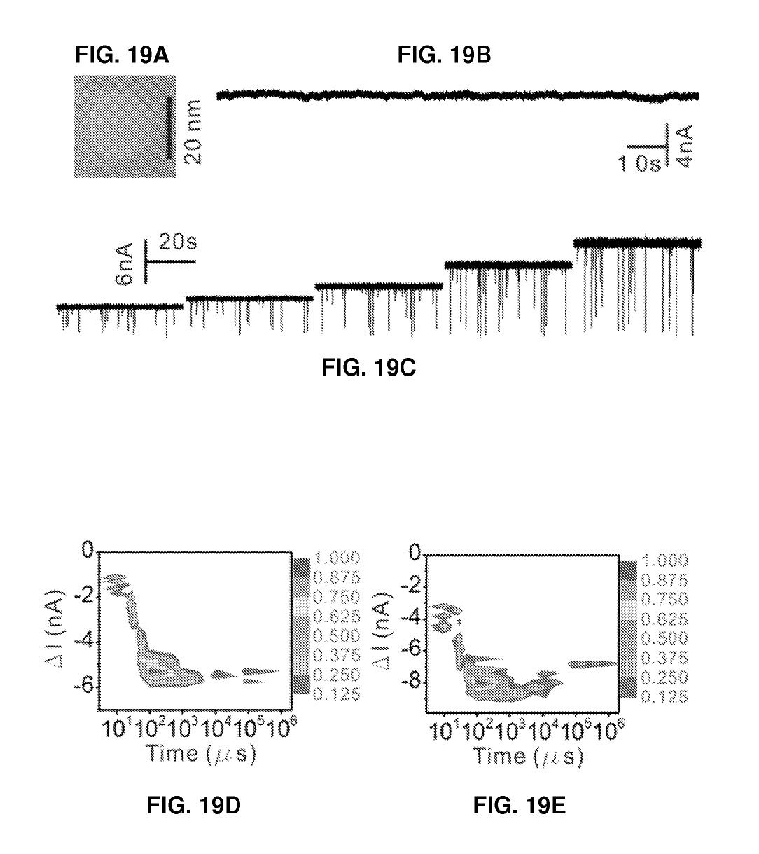

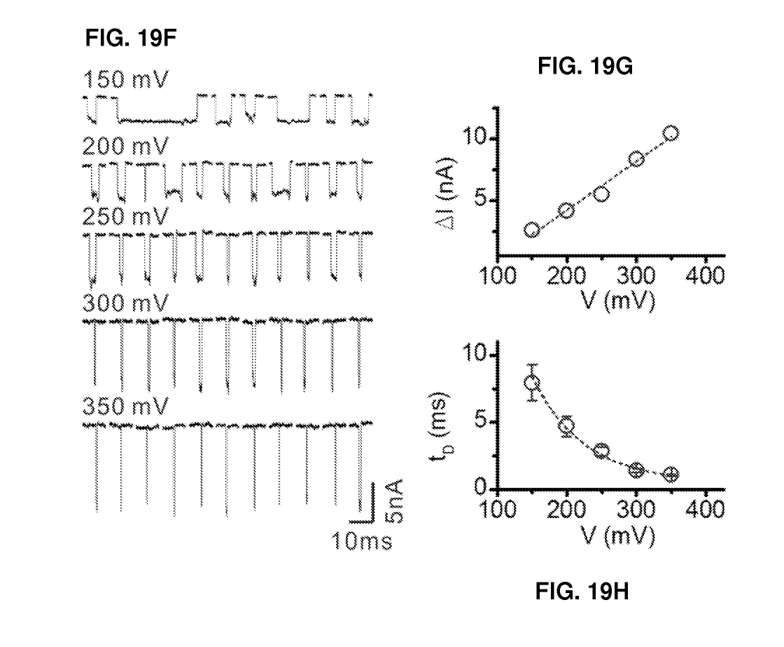

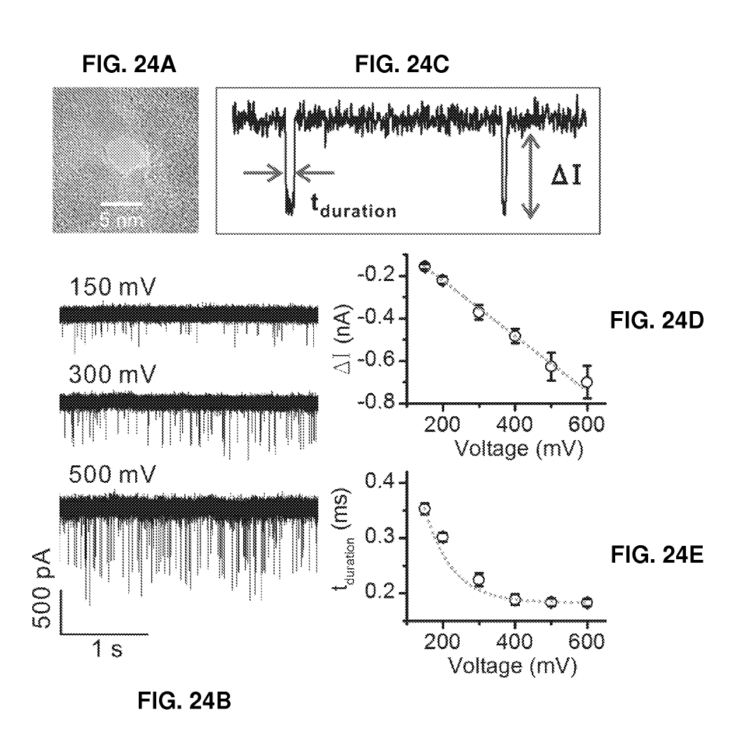

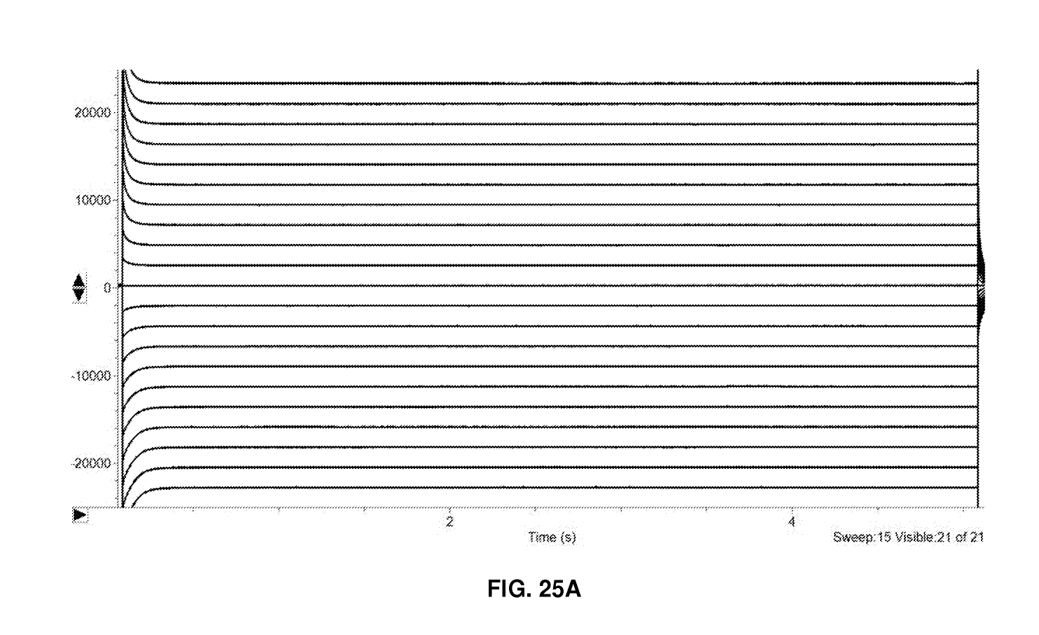

Selective Detection of Hypermethylation: A Nanopore relatively larger than the dimension of a methylated DNA fragment fully bound with MBD1x is utilized for the selective detection of hyMethDNA/MBD1x. The transmission electron microscopy (TEM) image of a 19 nm nanopore fabricated in a 10 nm thick SiN membrane (Norcada, Alberta, Canada) is shown in FIG. 19A. A 10 nM concentration of 90 bp unMethDNA was introduced in the nanopore for investigation of single-molecule translocation through a 19 nm nanopore. In this large nanopore, the ionic signature of DNA-only transport was not observed, unlike typical dsDNA transport through a smaller diameter nanopore, as shown in FIG. 24A-24E. The Nanopore ionic current signature of unnoticeable unMethDNA transports recorded at 200 mV is shown in FIG. 19B.

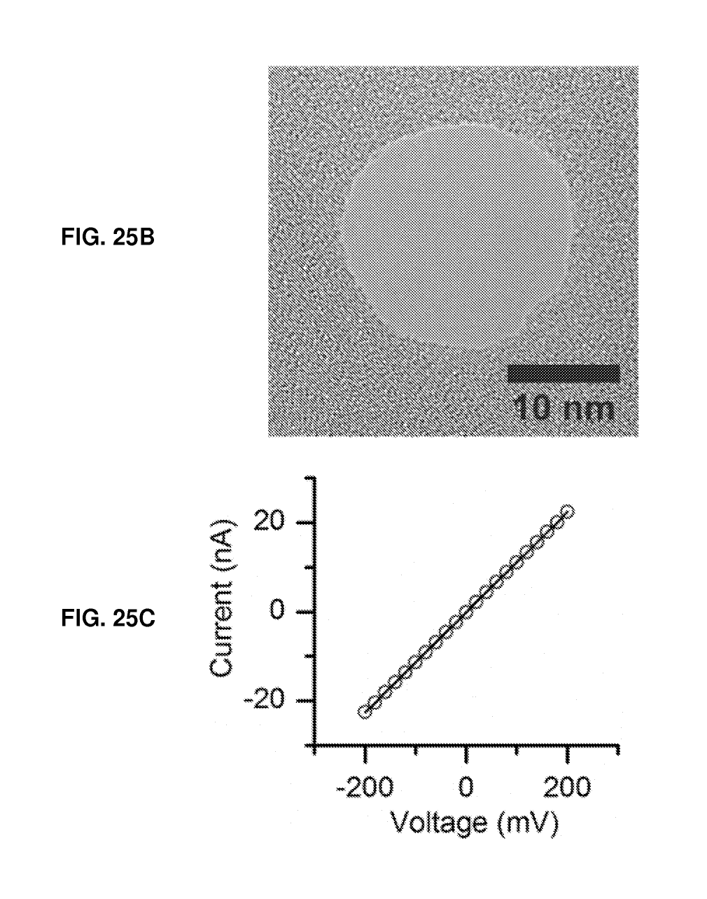

In contrast, a series of significant nanopore current blockades are observed after adding a mixture containing 100 pM of hyMethDNA/MBD1x complex and 10 nM unMethDNA to the nanopore. The selective detection of hyMethDNA/MBD1x complex over unMethDNA through a 19 nm nanopore can be explained by rapid translocation velocity and a largely unoccupied nanopore with unMethDNA. Smeets et al. demonstrated translocation of 5k and 48.5k dsDNAs through a 24.2 nm nanopore, and the translocation velocity of those molecules was obtained at 0.173 and 0.039 μs/bp, respectively.⁵⁰ The calculated translocation duration of 90 bp according to the velocity from these previous studies is ˜9.54 μs/molecule, which is undetectable from our recording sampling rate at 10 μs. Also, the current blockade of dsDNA of 2.2 nm diameter in a 19 nm nanopore is calculated to only be 1.2%, using the equation ΔI=(a/d)², where a and dare diameters of the molecule and the nanopore, respectively. With about 20 nA open pore current with about 500 pA peak-to-peak baseline noise (FIG. 25A-25C), the calculated current blockade of dsDNA at about 240 pA is clearly undetectable.

Meanwhile, the relatively larger diameter of the hyMethDNA/MBD1x complexes induces significant current blockade with larger blocked current during a prolonged translocation. Similar findings were reported with a RecA protein-coated dsDNA filament versus dsDNA alone.⁵⁰ Due to an undetectable quick and shallow nanopore ionic current blockade of unMethDNA, the nanopore exclusively detected hyMethDNA bound to MBD1x in the mixture with unmethylated DNA. Also, we have shown that unbound MBD1x is positively charged at pH 7.6 of nanopore buffer solution, thus transport of unbound MBD1x is not observed at positive driving voltage across the nanopore.³³ Consequently, a 19 nm nanopore can selectively detect translocation of the complexes and can screen the presence of methylated DNA in mixed sample solution. Representative long-term recordings of current blockades induced by transport of hyMethDNA/MBD1x complexes from 150 to 350 mV are shown in FIG. 19C from left to right. Contour plots of complex transport events at 250 and 300 mV are shown in FIGS. 19D and 19E, respectively. The wide spread of the current blockade in contour plots may be explained by unsuccessful DNA threading attempt,⁵¹ and by differing levels of methylation in single dsDNA molecules, as shown in a gel shift assay (FIG. 23 and study³³). However, the majority of current blockades fall into one group, indicating that most events involve complex transport and most complexes contain a fairly equal number of MBD1x. The representative transport events of single-molecule hyMethDNA/MBD1x are shown in FIG. 19F.

The analyses of hyMethDNA/MBD1x complex transport through a 19 nm nanopore are presented in FIGS. 19G-19H, for transport current blockade and transport duration. Values of current blockades were obtained by fitting the histogram of all blocked currents induced at each applied voltage to a Gaussian function, and the values of translocation duration were obtained by fitting the histogram of all blocked currents' duration to an exponential decay function. The short dashed trend line of current blockade values is fitted with a first-order polynomial function, indicating that conductance blockades increase at higher applied voltages. The short dashed trend line of transport duration values is fitted with an exponential decay function, indicating that the transport velocity is voltage-dependent. In summary, hypermethylated 90 bp DNA was specifically labeled with MBD1x, and the presence of hyMethDNA in a mixture with unMethDNA was selectively detected at the single-molecule level using a 19 nm diameter solid-state nanopore. This method can find applications in screening for the presence of hypermethylated DNA in a mixture.

Differentiation of hypermethylation from unmethylated DNA: The methylation patterns of human genomic DNA has recently been detected by collecting DNA on MBD chromatography columns after digesting methylated DNA into fragments with the restriction enzyme Msel.⁵² Herein, we further demonstrate the detection of hypermethylation using 30, 60, and 90 bp dsDNA. The hyMethDNA fragments contained 10% methylated CpGs uniformly distributed along the entire sequence, while unMethDNA fragments possessed no methylation. Nanopores with diameters ranging from 7.1 to 9.5 nm are utilized to detect methylated dsDNA. We demonstrated the discrimination of hypermethylated and nonmethylated DNA fragments.

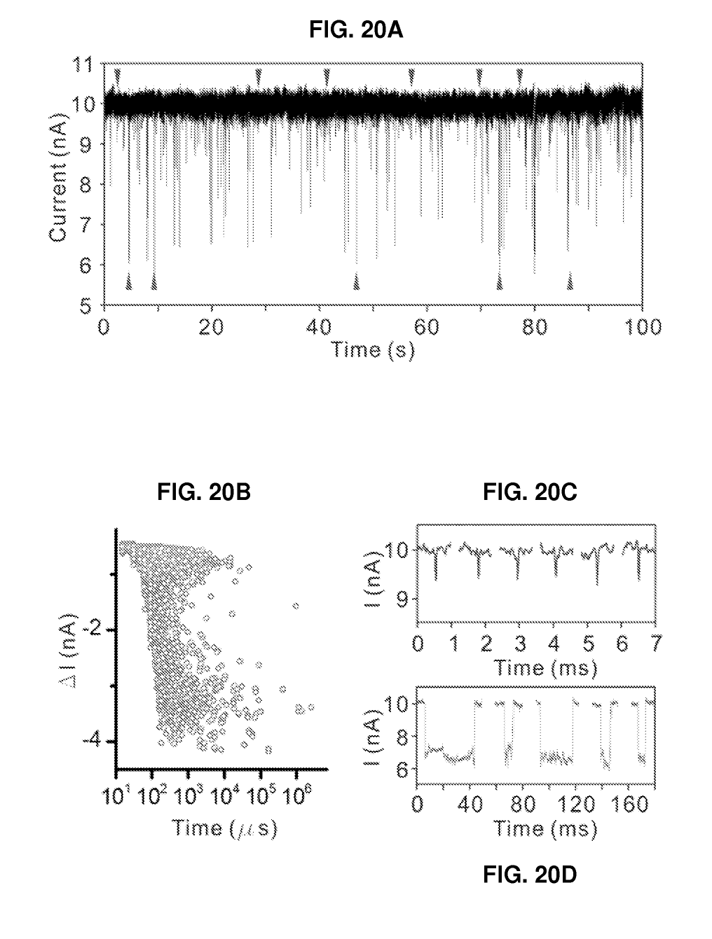

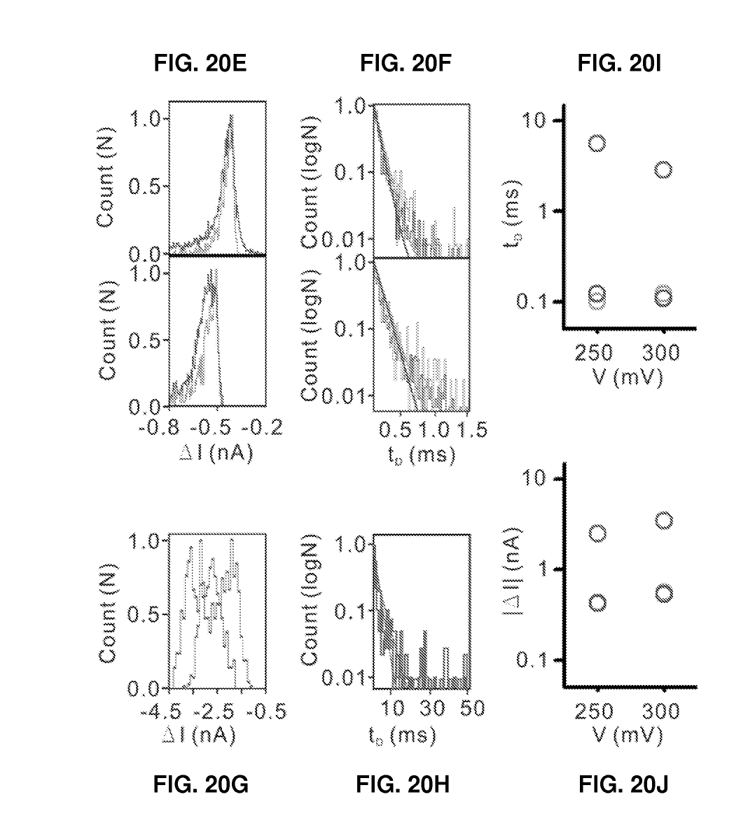

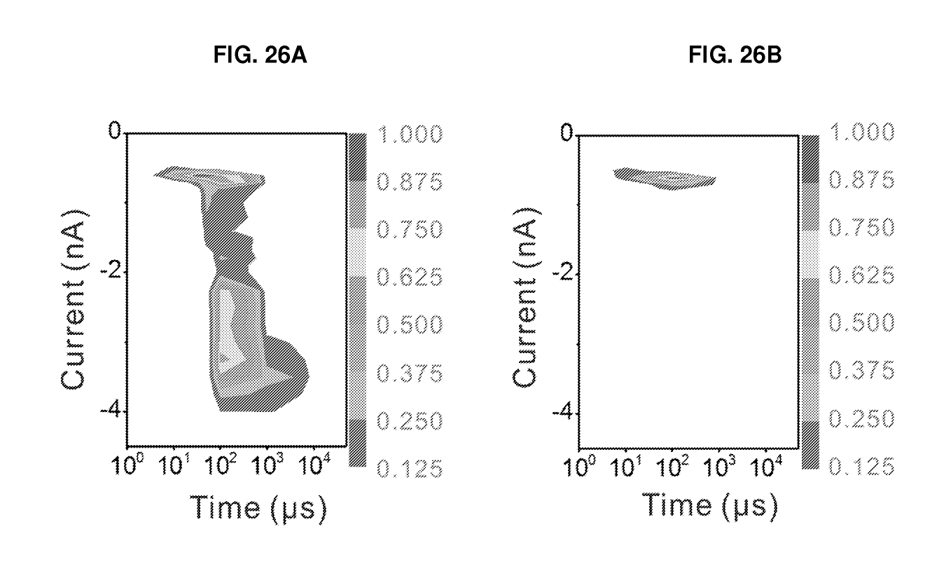

First, 90 bp dsDNA fragments in a mixture (100 pM for both hyMethDNA and unMethDNA) were analyzed through a 7.7 nm diameter nanopore. The nanopore ionic current in FIG. 20A shows mixed transport events of 90 bp hyMethDNA (fully bound with MBD1x) and unMethDNA recorded at 300 mV. The nanopore with diameter comparable with the dimension of hyMethDNA/MBD1x complex clearly detected transport events of the unMethDNA and hyMethDNA/MBD1x. The cross-sectional diameter of hyMethDNA/MBD1x was about 5 nm when a single protein bound to DNA and about 7.6 nm with multiple bound proteins, as also shown in a previous study.³³ The scatter plot of all mixed single-molecule transport events is shown in FIG. 20B and presents prolonged-deeper current blockade of hyMethDNA/MBD1x transports (FIG. 20C) along with fast-shallow current blockage from transport of unMethDNA (FIG. 20D). A contour plot of FIG. 20B is provided to show two major distinct event populations for naked DNA and the DNA complex transports (FIG. 26-26B). In comparison with the scatter plots of mixed events through the 19 nm nanopore shown in FIGS. 19D-19E, unMethDNA and hyMethDNA/MBD1x are clearly discriminated using the 7.7 nm nanopore: the shallow current blocking events from unMethDNA and deep current blocking events from hyMethDNA/MBD1x. To confirm that the fast-shallow events in the mixture are the single-molecule transport of unMethDNA, a separate investigation of unMethDNA single-molecule transport through the same nanopore was performed and a scatter plot of pure unMethDNA transport events is superimposed on the scatter plot of mixed events. The analysis of separate unMethDNA transport and fast-shallow events in mixed molecule transport showed good agreement in current blockades and transport durations. Histograms of transport durations and current blockades were obtained from mixtures and separate unMethDNA current traces recorded at 250 and 300 mV, as shown in FIGS. 20E-20F. The values of transport duration of unmethylated DNA were obtained by fitting the transport duration histogram to an exponential function. Both transport durations of unMethDNA and fast-shallow events in mixed solution ranged between 100 and 125 μs at 250 and 300 mV.

Current blockades are obtained by fitting the current blocking of events to a Gaussian function. Single-molecule transport of unMethDNA blocked a current of 0.433 nA at 250 mV and 0.561 nA at 300 mV, and fast-shallow events blocked a current of 0.429 nA at 250 mV and 0.537 nA at 300 mV. Consequently, the fast-shallow events in mixed solution represent single-molecule transport of unMethDNA through the nanopore rather than collisions of the complex at the entrance of the solid-state nanopore. Representative nanopore electrical signatures of single-molecule unMethDNA transport and single-molecule hyMethDNA/MBD1x complex transport in mixed events are shown in FIGS. 20C-20D. The analysis of hyMethDNA/MBD1x single-molecule transport events showed about 2.5 and about 3.5 nA current blocking, obtained by fitting the histogram in FIG. 20G to a Gaussian function. The analysis also showed 5.59 and 2.86 ms transport duration at 250 and 300 mV, obtained by fitting the histogram in FIG. 20H to an exponential decay function. The comparison between hyMethDNA/MBD1X and unMethDNA is shown in FIG. 20I for transport times and FIG. 20J for current blockades. A hypermethylated DNA bound with MBD1x is clearly distinguishable from the signatures of the unMethDNA events.

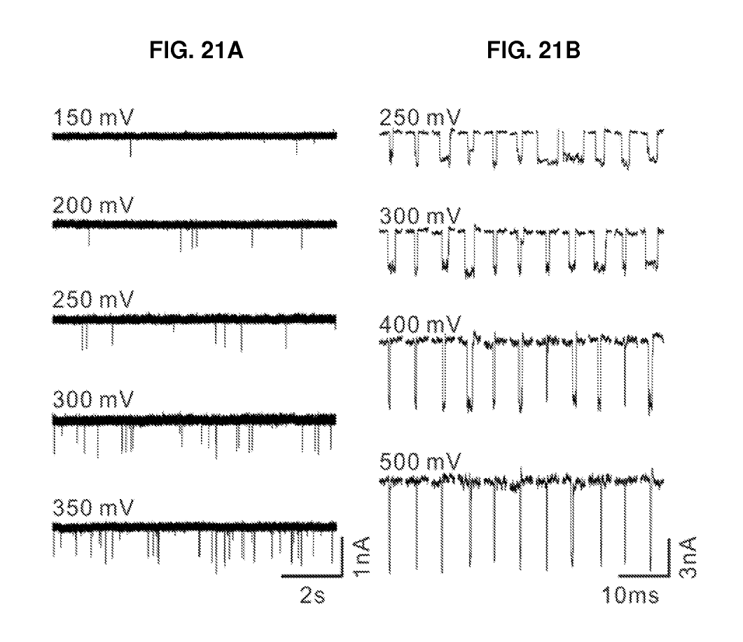

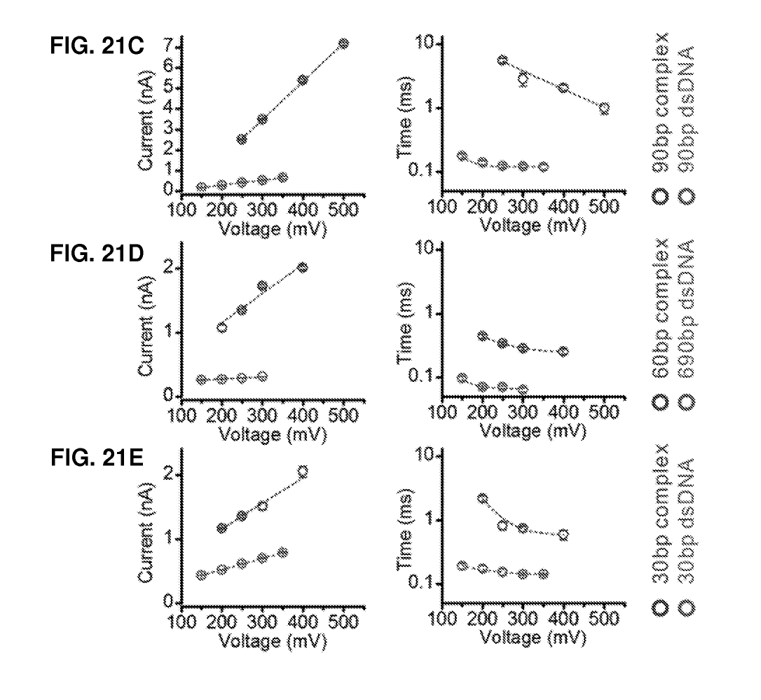

Various length DNA fragments are also used to discriminate 10 pM of hyMethDNA fully bound with MBD1x in 1 nM of unMethDNA through nanopore ionic signatures of current blockage and duration. Representative current traces of unMethDNA and sample events of hyMethDNA/MBD1x are shown in FIGS. 21A-21B. Analyses of single-molecule transport of unMethDNA and hyMethDNA/MBD1x are compared in FIGS. 21C-21E for 90, 60, and 30 bp DNA fragments. In each panel, the left graph shows the current blockade difference and the right graph shows the transport duration difference between unMethDNA (in purple) and hyMethDNA/MBD1x complex (in brown). The trend line of current blockades is fitted by a first-order polynomial function, and the trend line of transport times is fitted with an exponential decay function. These trends are shown as short dashed lines in FIGS. 21C-21E and are consistent with previous findings where conductance blockades of DNA translocation increase in depth at increased applied voltages^{33,53} and reduce in duration in a voltage-dependent manner as applied voltage increases.⁵¹ Specifically at 300 mV, 90 bp hyMethDNA/MBD1x was discriminated from 90 bp unMethDNA by a 6.5-fold difference in current blocking and a 23-fold difference in transport duration; 60 bp hyMethDNA/MBD1x demonstrated 5.5-fold current blocking and 4.5-fold transport duration over 60 bp unMethDNA, and 30 bp hyMethDNA/MBD1x demonstrated 2.1-fold current blocking and 5.1-fold transport duration as compared to 30 bp unMethDNA.

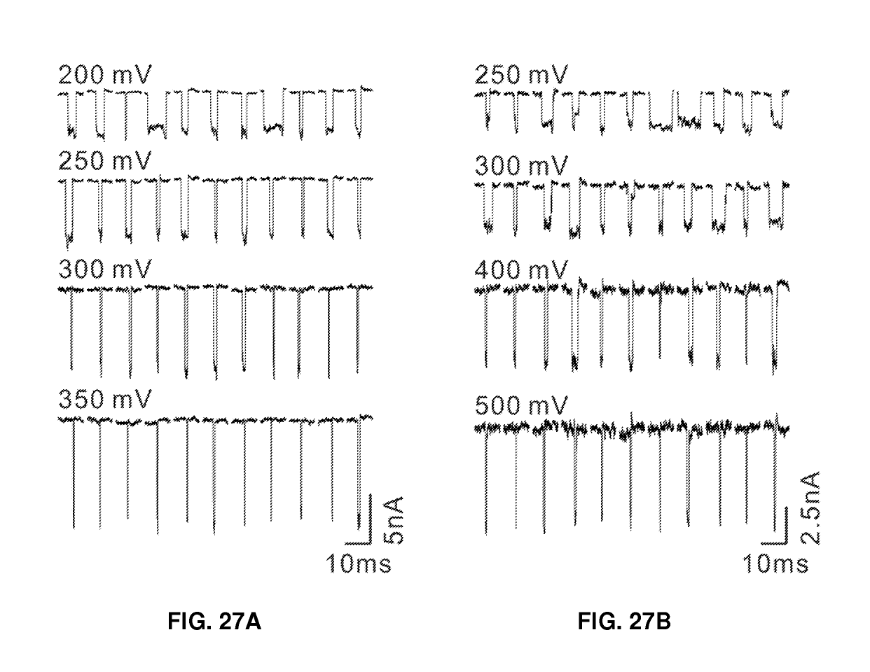

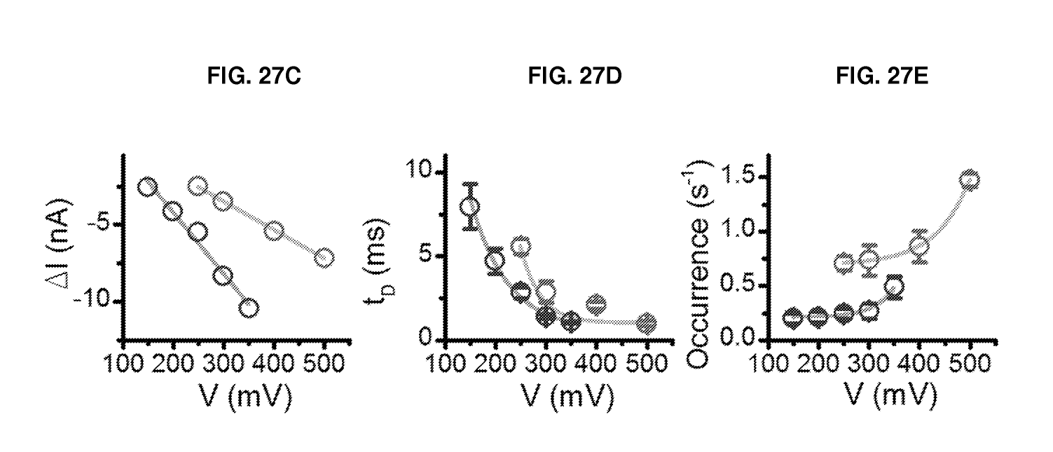

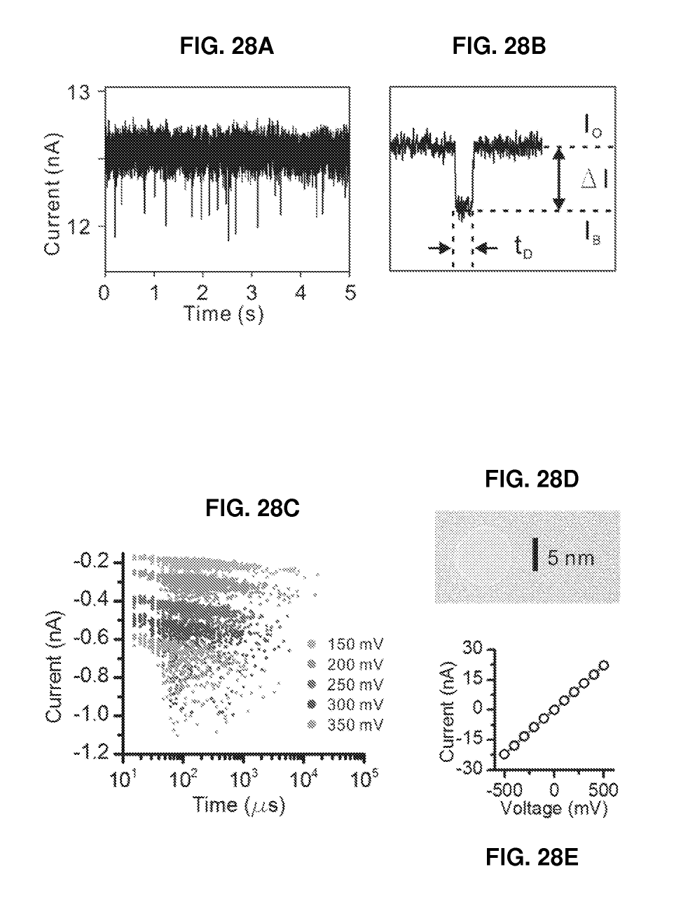

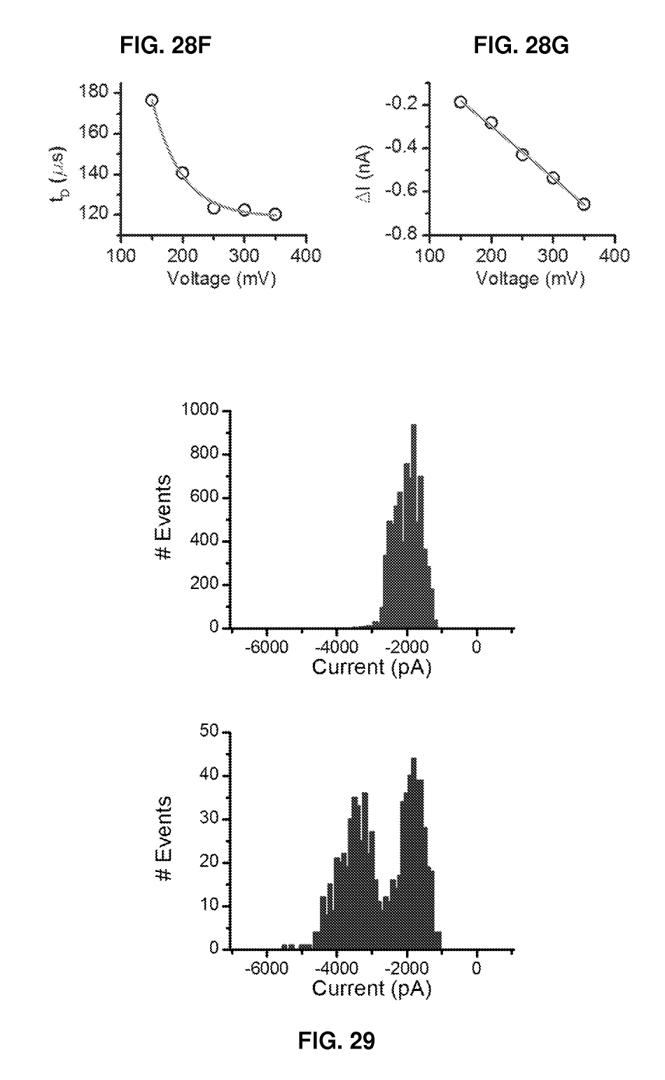

The comparison of single-molecule transport events between complex and unMethDNA recorded at 300 mV is shown in Table 1. Interestingly, the 90 bp hyMethDNA with 10 MBD1x shows significantly prolonged transport times compared to 30 bp hy-MethDNA with 3 MBD1x through nanopores of similar diameters (see Table 1). Interaction between MBD1x (on the DNA) and the surface of a nanopore with the opposite charge was reported to slow the translocation of hyMethDNA/MBD1x complexes through a nanopore.³³ Consequently, more MBD1x-associated DNA has longer transport time. To confirm this interaction, single-molecule transport events of 90 bp hyMethDNA fully bound with MBD1x through 19 and 7.7 nm nanopores are also compared (FIG. 27A-27E). Transport durations of hyMethDNA/MBD1x through 7.7 nm are 5.59 and 2.86 ms and through the 19 nm pore are 2.83 and 1.43 ms at 250 and 300 mV, respectively. Stronger interactions between the protein and the surface of the narrow nanopore (7.7 nm) slow the translocation durations of complexes by 2-fold compared to the larger nanopore (19 nm) at 250 and 300 mV.

Detection of a CpG Dyad in Short dsDNA: The patterns of DNA epigenetic alterations in cancer vary from the individual CpG dyad at the local level to methylations in 1 million base pairs, or DNA demethylation during carcinogenesis which results in loss of methylation on both strands via possible intermediates of hemimethylated dyads.⁵⁴ Although reduced methylation in DNA (hypomethylation) compared to a normal level is another major epigenetic modification in cancer cells, diagnosis of DNA hypomethylation using conventional techniques such as methylation-specific PCR is technically limited and challenging.⁵⁵ Herein, a nanopore-based methylation assay demonstrates detection of reduced methylation at the local level single CpG dyad in the DNA fragment. We utilize KZF to detect local methylation in DNA fragments with its relevance to cancer and high binding affinity to methylated CpGs. KZF demonstrates high binding affinity of K_{d}=210 (±50 pM, forming 1:1 complexes with single consecutive methylated CpGs,⁴⁵ and is reported to bind and silence aberrantly methylated DNA repair genes and tumor suppression in cancer cells.⁵⁶ We select two repetitive methylated CpGs to mimic the methylation pattern of hypomethylation occurring in normally methylated CpG islands in somatic tissues.⁵⁷

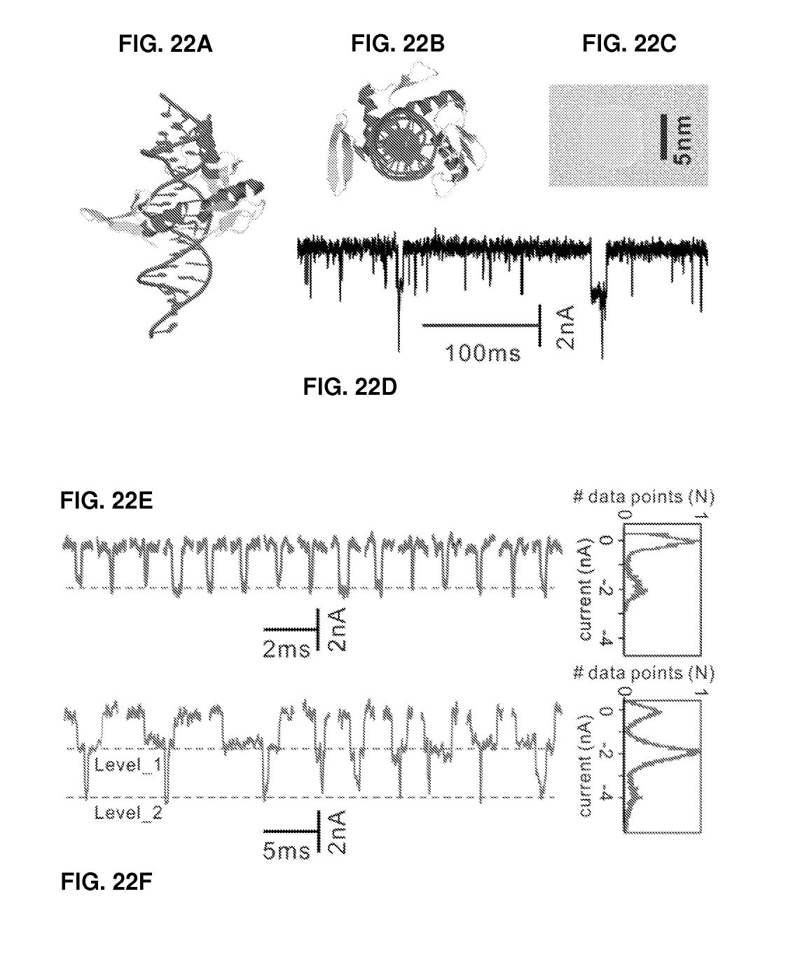

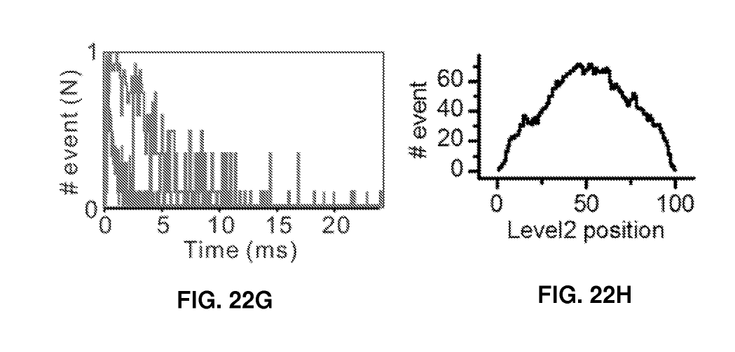

The target 90 bp loMethDNA fragments have 30 potential CpG methylation sites, but only two repetitive CpG sites at the center are methylated. The target fragments are also designed to have repeated sequences to mimic the hypomethylation occurring in repeated sequences of genomic DNA.⁵⁴ The crystal structure of engineered KZF bound on DNA methylated sites is shown FIG. 22A (side view) and FIG. 22B (top-down view).⁴⁵ This loMethDNA bound with KZF is discriminated from unMethDNA with different nanopore ionic current events. We utilize a nanopore for which the diameter tightly fits with the width of loMethDNA/KZF complex. The width of the complex is 4.9 nm, and the diameter of the nanopore used was 5.5 nm, as shown in FIGS. 22B-22C. The nanopore current trace of loMethDNA/KZF at 10 pM mixed unMethDNA at 1 nM and is shown in FIG. 22D, showing significantly distinct current blockades. A representative nanopore electrical signature of single-molecule un-MethDNA transport and an all-point histogram of transport events are shown in FIG. 22E, and current events of loMethDNA/KZF transport are shown in FIG. 22F, with the all-point histogram in the right panel. Current blockade histograms with all events are presented in FIG. 29).

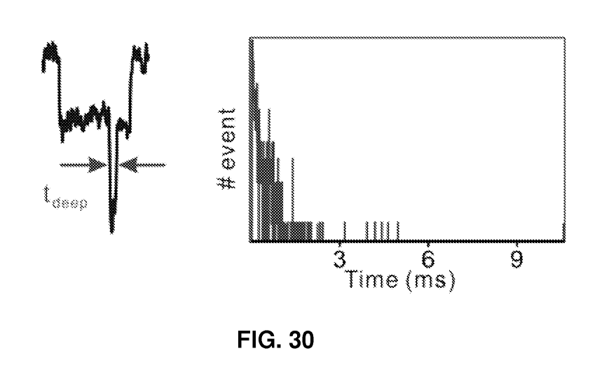

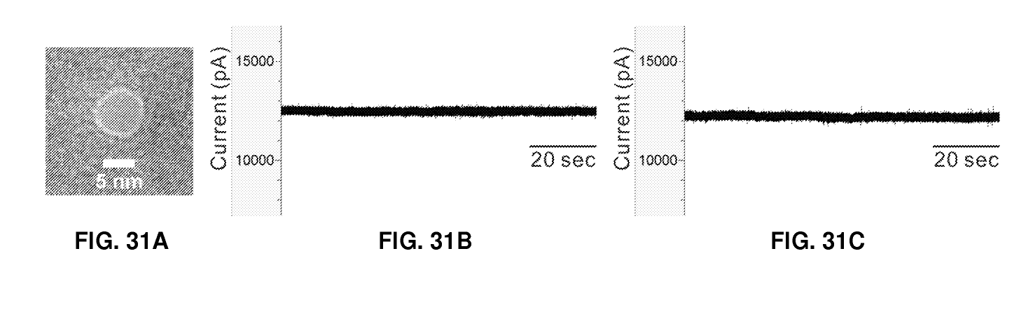

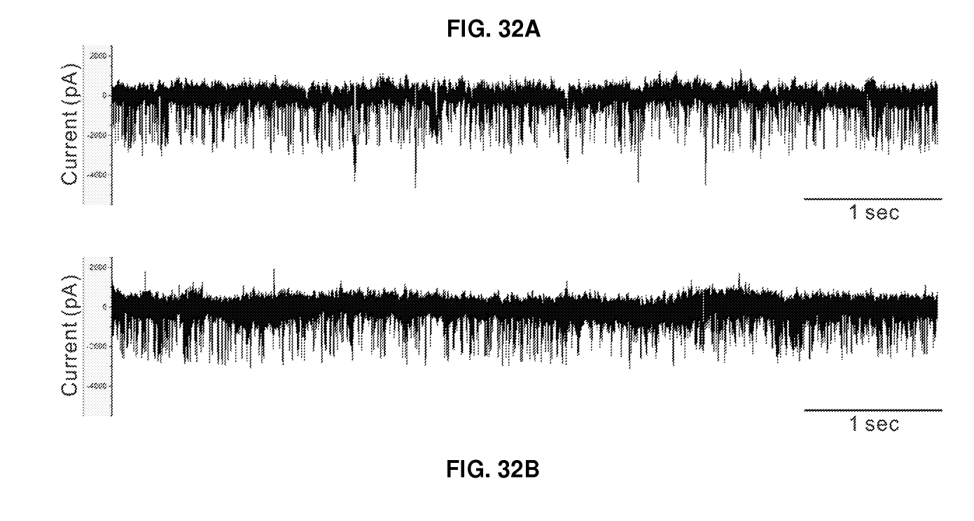

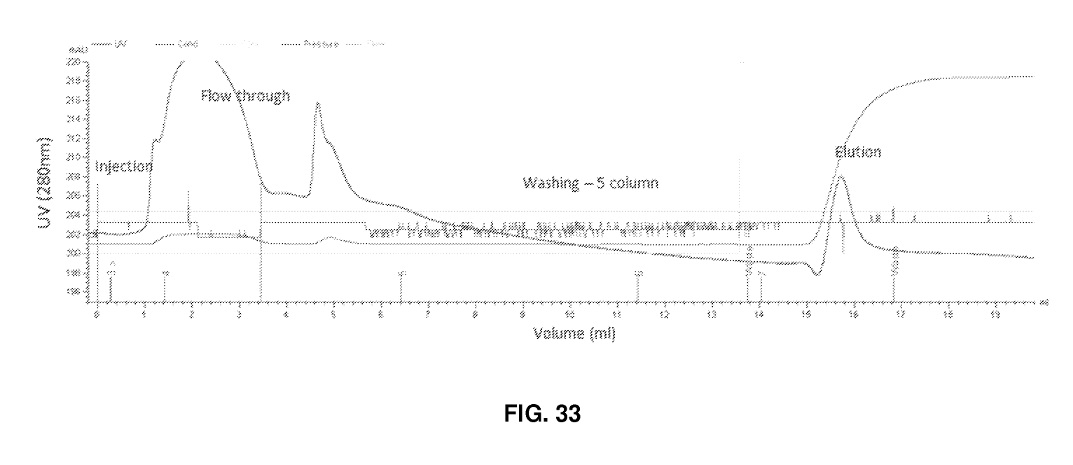

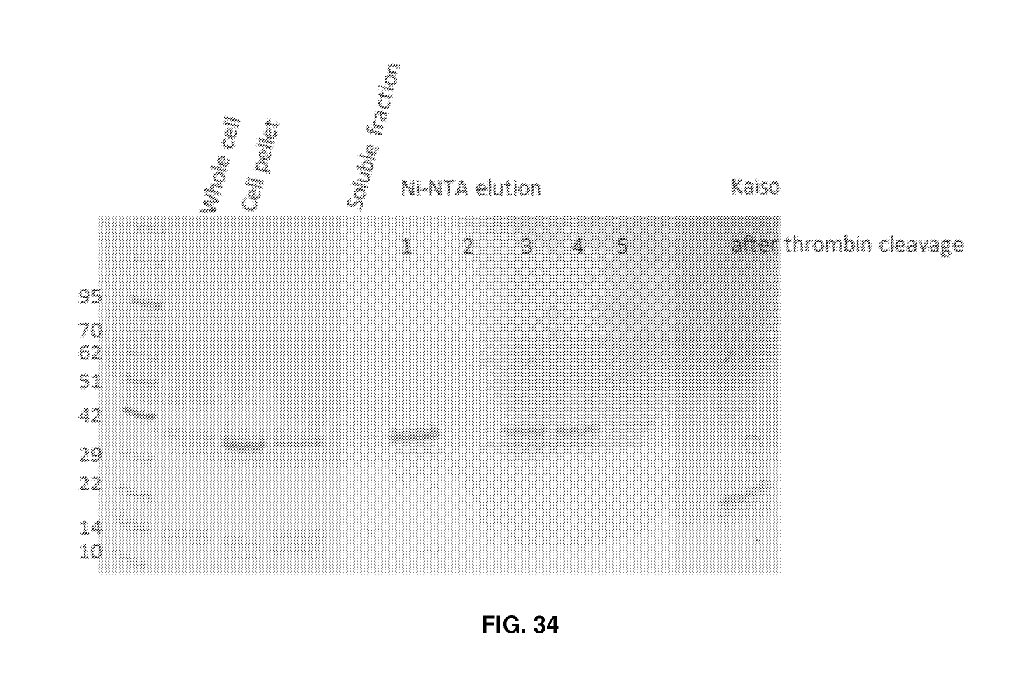

Current blockade of the loMethDNA/KZF complex showed two distinct levels; the shallow current blockade of about 2 nA is attributed to transport of the DNA-only region of the complex, and the deeper blockage of about 4 nA is attributed to the region of DNA bound with KZF in complex. The peak of the shallow current blockade in the all-point histogram in FIG. 22F is well matched with the peak current blockade of unMethDNA transport in FIG. 22E. Hence, the shallow blocking in loMethDNA/KZF can be attributed to the translocation of a protein-free DNA region in the complex through the nanopore. Our nanopore-based methylation assay discriminates loMethDNA bound with KZF at 2-fold current blockade and 5-fold transport duration from unMethDNA. Current blockade of unMethDNA was obtained at 1.87 (±0.02 nA, and loMethDNA/KZF was at 3.77 (±0.03 nA. The histograms of transport duration of both unMethDNA and loMethDNA-MBD1x are shown in FIG. 22G, and the fitted values of transport times from an exponential decay function are obtained at 0.19±0.006 and 3.98±0.32 ms, respectively. In addition, FIG. 22F shows a stepwise current blockade with two current blocking levels. Level_2 current blockade was clearly distinguished from level_1, and solely obtained level_2 duration was at 0.33±0.014 ms (FIG. 30). The occurrence of level_2 current blockade was mainly observed at the center of the whole complex transport, as shown in FIG. 22H. The x-axis represents the length of entire complex transport, normalized and recalculated as 100%. The peak occurrence of deeper current blockade was obtained by fitting a Gaussian function to the occurrence histogram, and the fitting value was 52.1%, indicating that a deeper current blockade mainly occurs at the middle of the entire complex translocation. These results provide evidence that the position of methylated CpGs in loMethDNA can be profiled by analyzing the location of level_2 current blocking from the entire stepwise DNA complex translocation.

In summary, we utilize KZF to detect loMethDNA and to roughly determine the methylation location where the nanopore electrical current signature of loMethDNA/MBP demonstrated stepwise deeper current blocking, as shown in FIG. 22F. This was significantly different from the prolonged single level deeper current blocking of hyMethDNA/MBP in FIG. 19F and FIG. 20C. Interestingly, KZF also has high binding affinity for symmetric single methylated CpG dinucleotides and hemimethylation of two adjacent CpGs in dsDNA with slightly reduced binding affinity.⁴⁵ With the versatile binding affinity of KZF to various methylation patterns, various patterns can be screened using the nanopore-based methylation assay provided herein.

This example is a direct electrical analysis technique to detect various methylation levels on DNA fragments at the single-molecule level using solid-state nanopores. Hypermethylated DNA, a molecular-level epigenetic biomarker for cancer, can be selectively labeled using MBD1x as a methylation-specific label and can be detected without the need for any further processes, such as bisulfite conversion, tagging with fluorescent agent, or sequencing. The large nanopore successfully exhibited exclusive detection of methylated DNA bound to MBD1x in a mixture with unmethylated DNA. This method has an initial application for screening the presence of hypermethylated DNA. Differentiation between hypermethylated and unmethylated dsDNA oligos is demonstrated using sub-10 nm nanopores, thus nanopore-based methylation assays also have the potential to identify abnormally methylated DNA in clinical tests aimed at diagnosis of diseases such as cancer. Hypomethylation in locally methylated CpG dyads is another epigenetic biomarker for cancer, and the methylated CpG dyads were labeled with KZF and discriminated from unmethylated DNA_hypomethylated DNA in this case. Furthermore, we can profile the methylation position in DNA. However, a nanopore-based methylation assay mproves the efficiency for low sample volume obtained from body fluids. Next steps include integrating a nanopore-based assay in a microfluidic system to collect genomic DNA samples adjacent to the nanopore and detect methylation in situ.

Bodily fluids, such as stool or blood, represent rich sources of genomic DNA that can be obtained noninvasively. DNA sequences can be hybrid-captured from such samples and concentrated near a nanopore integrated with a microfluidic system. Wanunu et al. showed successful nanopore detection of 1000 events in 15 min with a sample amount of 1 000 000 molecules/10 μL.⁵⁸ The relative percentage of aberrantly methylated DNA in stool samples from patients with colorectal cancer averages about 5% but can be much lower in some instances.⁵⁹ Using the approaches presented in this example, the nanopore-based methylation detection method may be used to develop a new methylation assay from small volume samples. This is a fundamental improvement and provides a rapid, accurate, and amplification-free methylation detection platform.

Solid-State Nanopore, Chemicals, and Materials: The free-standing low-stress SiN membranes with 10 nm thickness and 50×50 μm² area, supported on a silicon substrate, were purchased from Norcada (Alberta, Canada). Single nanopores with various diameters were drilled with condensed electron beam using a JEOL 2010F field emission transmission electron microscope.

All custom DNA fragments including methylation patterns for nanopore experiments are synthesized and purchased from Integrated DNA Technologies (Coralville, Iowa). The nanopore measurements are performed in 1 M KCl at pH 7.6 containing 10 mM Tris and 1 mM ethylenediaminetetraacetic acid (EDTA) for hypermethylated DNA fragments bound with MBD1x and in 0.2 M NaCl at pH 7.6 containing 10 mM Tris and 1 mM EDTA for locally methylated DNA fragments bound with KZF. The methylated DNA/MBP complexes were prepared and incubated for 15 min at room temperature (25±2° C.) immediately before the nanopore experiment. Hypermethylated DNA was mixed with MBD1x in 80 mM KCl at pH 7.6 containing 10 mM Tris, 1 mM EDTA, and 0.4 mM DTT. The high ratio of MBD1x to methylated DNA is used to fully bind MBD1x to methylated DNA: ratio of 6:1 for 30 bp, 12:1 for 60 bp, and 20:1 for 90 bp methylated DNA. Locally methylated DNA and KZF are mixed in equal ratio in 200 mM NaCl at pH 7.6 containing 10 mM Tris, 1 mM ZnCl, and 1 mMTCEP.

Nanopore Electrical Measurements: Nanopore chips are piranha-cleaned (two-thirds of 95% H₂SO₄ and one-third of 30% H₂O₂) for 10 min and thoroughly rinsed five times with large amount of deionized H₂O, and then the nanopore chip clamped and sealed between two custom acrylic chambers to form the nanopore, the only electrical path of ions between the two reservoirs. Ag/AgCl electrodes were immersed in reservoirs for ionic current recordings. Axopatch 200B was used for applying potentials and measuring currents, and data were recorded using a Digidata 1440A data acquisition system. Nanopore current traces were recorded using a 10 kHz built-in low-pass Bessel filter and 10 μs sampling rates. Instrumental control and data analysis were performed using Clampex 10.2 and Clampfit 10.2. All data points of current blockage were obtained using Gaussian fit, and transport duration was determined using an exponential decay function in Clampfit 10.2 software. Also, all error bars are given with standard error obtained during the fitting. All nanopore experiments were performed in a dark double Faraday cage on an antivibration table at room temperature (25±2° C.).

MBD1x Protein Purification: MBD1x purification is outlined in a previous report.³³

Plasmid Construction: The Kaizo zinc finger DNA sequence is codon-optimized, PCR-amplified, and cloned into pUC19 (Fisher). The pUC19 plasmid is digested with Xma1 and subcloned into pQE80L (Quiagen) expression vector that is modified to contain mCherry and a thrombin cleavage site⁶⁰ and digested with Xma1 (New England Biolabs) and calf intestinal alkaline phosphatase (New England Biolabs). The expression vector is transformed into DH5-alpha Escherichia coli, and positive colonies are checked by sequencing performed at the UIUC core sequencing facility.

KZF Protein Expression: The pQE80L expression vector containing mCherry-KZF is transformed into E. coli BL21 (DE3)pLysS. An overnight culture of a single colony was grown in Luria-Bertani medium with ampicillin (100 μg/L). The culture was expanded into 1 L of Luria-Bertani broth with ampicillin, and at OD₆₀₀ of 0.3, isopropyl-D-thiogalactopyranoside (1.0 mM) was added to the culture. Cell pellets were harvested by centrifugation at 6000 g for 15 min at 4° C. and snap frozen.

KZF Protein Purification: Lysis buffer (20 mM Tris at pH 7.9, 0.1 mM ZnCl₂, 8 M urea, 10% v/v glycerol, 500 mM NaCl, 10 mM imidazole) was added to the cell pellet and incubated with lysozyme (1 mg/mL) at 4° C. for 1 h. The lysate was sonicated and then centrifuged at 10 000 g at 4° C. for 1 h. The bacterial supernatant was added to a column packed with Ni-NTA resin for 1 h at 4° C. The column was extensively washed with wash buffer (20 mM Tris at pH 7.9, 0.1 mM ZnCl₂, 10% v/v glycerol, 500 mM NaCl, 20 mM imidazole), and mCherry was cleaved by incubation with biotinlyated thrombin overnight at 4° C. Excess biotinylated thrombin was removed by streptavidin-coated beads and centrifugation. Protein was diluted in TDZ buffer (20 mM Tris at pH 7.9, 0.1 mM ZnCl₂, 20% v/v glycerol) and injected into heparin column in an AKTA FPLC (GE HealthCare). The column was washed with 5-10 volumes of TDZ buffer with 200 mM NaCl, and the protein was eluted with TDZ buffer with q1 M NaCl; 70% glycerol was added, and the purified KZF protein was stored at −20° C.

### REFERENCES FOR EXAMPLE 2

1. Fearon, E. R.; Vogelstein, B. A Genetic Model for Colorectal Tumorigenesis. Cell 1990, 61, 759-767.
2. Baylin, S. B.; Herman, J. G.; Graff, J. R.; Vertino, P. M.; Issa, J. P. Alterations in DNA Methylation: A Fundamental Aspect of Neoplasia. Adv. Cancer Res. 1998, 72, 141-196.
3. Li, E.; Bestor, T. H.; Jaenisch, R. Targeted Mutation of the DNA Methyltransferase Gene Results in Embryonic Lethality. Cell 1992, 69, 915-926.
4. Laird, P. W. The Power and the Promise of DNA Methylation Markers. Nat. Rev. Cancer 2003, 3, 253-266.
5. Laird, P. W.; Jaenisch, R. The Role of DNA Methylation in Cancer Genetics and Epigenetics. Annu. Rev. Genet. 1996, 30, 441-464.
6. Esteller, M.; Corn, P. G.; Baylin, S. B.; Herman, J. G. A Gene Hypermethylation Profile of Human Cancer. Cancer Res. 2001, 61, 3225-3229.
7. Das, P. M.; Singal, R. DNA Methylation and Cancer. J. Clin. Oncol. 2004, 22, 4632-4642.
8. Iqbal, S. M.; Akin, D.; Bashir, R. Solid-State Nanopore Channels with DNA Selectivity. Nat. Nanotechnol. 2007, 2, 243-248.
9. Strathdee, G.; Brown, R. Aberrant DNA Methylation in Cancer: Potential Clinical Interventions. Expert Rev. Mol. Med. 2002, 4, 1-17.
10. Vanaja, D. K.; Ehrich, M.; Van den Boom, D.; Cheville, J. C.; Karnes, R. J.; Tindall, D. J.; Cantor, C. R.; Young, C. Y. Hypermethylation of Genes for Diagnosis and Risk Stratification of Prostate Cancer. Cancer Invest. 2009, 27, 549-560.
11. Adorjan, P.; Distler, J.; Lipscher, E.; Model, F.; Muller, J.; Pelet, C.; Braun, A.; Florl, A. R.; Gutig, D.; Grabs, G.; et al. Tumour Class Prediction and Discovery by Microarray-Based DNA Methylation Analysis. Nucleic Acids Res. 2002, 30, e21.
12. Iacobuzio-Donahue, C. A.; Maitra, A.; Olsen, M.; Lowe, A. W.; Van Heek, N. T.; Rosty, C.; Walter, K.; Sato, N.; Parker, A.; Ashfaq, R.; et al. Exploration of Global Gene Expression Patterns in Pancreatic Adenocarcinoma Using cDNA Microarrays. Am. J. Pathol. 2003, 162, 1151-1162.
13. Goelz, S. E.; Vogelstein, B.; Hamilton, S. R.; Feinberg, A. P. Hypomethylation of DNA from Benign and Malignant Human-Colon Neoplasms. Science 1985, 228, 187-190.
14. Feinberg, A. P.; Gehrke, C. W.; Kuo, K. C.; Ehrlich, M. Reduced Genomic 5-Methylcytosine Content in Human Colonic Neoplasia. Cancer Res. 1988, 48, 1159-1161.
15. Oshimo, Y.; Nakayama, H.; Ito, R.; Kitadai, Y.; Yoshida, K.; Chayama, K.; Yasui, W. Promoter Methylation of Cyclin D2 Gene in Gastric Carcinoma. Int. J. Oncol. 2003, 23, 1663-1670.
16. Akiyama, Y.; Maesawa, C.; Ogasawara, S.; Terashima, M.; Masuda, T. Cell-Type-Specific Repression of the Maspin Gene Is Disrupted Frequently by Demethylation at the Promoter Region in Gastric Intestinal Metaplasia and Cancer Cells. Am. J. Pathol. 2003, 163, 1911-1919.
17. Cho, M.; Uemura, H.; Kim, S. C.; Kawada, Y.; Yoshida, K.; Hirao, Y.; Konishi, N.; Saga, S.; Yoshikawa, K. Hypomethylation of the Mn/Ca9 Promoter and Upregulated Mn/Ca9 Expression in Human Renal Cell Carcinoma. Br. J. Cancer 2001, 85, 563-567.
18. Nakamura, N.; Takenaga, K. Hypomethylation of the Metastasis-Associated S100a4 Gene Correlates with Gene Activation in Human Colon Adenocarcinoma Cell Lines. Clin. Exp. Metastasis 1998, 16, 471-479.
19. Badal, V.; Chuang, L. S. H.; Tan, E. H. H.; Badal, S.; Villa, L. L.; Wheeler, C. A.; Li, B. F. L.; Bernard, H. U. Cpg Methylation of Human Papillomavirus Type 16 DNA in Cervical Cancer Cell Lines and in Clinical Specimens: Genomic Hypomethylation Correlates with Carcinogenic Progression. J. Virol. 2003, 77, 6227-6234.
20. De Capoa, A.; Musolino, A.; Della Rosa, S.; Caiafa, P.; Mariani, L.; Del Nonno, F.; Vocaturo, A.; Donnorso, R. P.; Niveleau, A.; Grappelli, C. DNA Demethylation Is Directly Related to Tumour Progression: Evidence in Normal, Pre-malignant and Malignant Cells from Uterine Cervix Samples. Oncol. Rep. 2003, 10, 545-549.
21. Sato, N.; Maitra, A.; Fukushima, N.; van Heek, N. T.; Matsubayashi, H.; lacobuzio-Donahue, C. A.; Rosty, C.; Goggins, M. Frequent Hypomethylation of Multiple Genes Overexpressed in Pancreatic Ductal Adenocarcinoma. Cancer Res. 2003, 63, 4158-4166.
22. Piyathilake, C. J.; Henao, O.; Frost, A. R.; Macaluso, M.; Bell, W. C.; Johanning, G. L.; Heimburger, D. C.; Niveleau, A.; Grizzle, W. E. Race- and Age-Dependent Alterations in Global Methylation of DNA in Squamous Cell Carcinoma of the Lung (United States). Cancer, Causes Control, Pap. Symp. 2003, 14, 37-42.
23. Zou, H. Z.; Taylor, W. R.; Harrington, J. J.; Hussain, F. T. N.; Cao, X. M.; Loprinzi, C. L.; Levine, T. R.; Rex, D. K.; Ahnen, D.; Knigge, K. L.; et al. High Detection Rates of Colorectal Neoplasia by Stool DNA Testing with a Novel Digital Melt Curve Assay. Gastroenterology 2009, 136, 459-470.
24. Kisiel, J. B.; Yab, T. C.; Taylor, W. R.; Chari, S. T.; Petersen, G. M.; Mahoney, D. W.; Ahlquist, D. A. Stool DNA Testing for the Detection of Pancreatic Cancer Assessment of Methylation Marker Candidates. Cancer 2012, 118, 2623-2631.
25. Kandimalla, R.; van Tilborg, A. A.; Zwarthoff, E. C. DNA Methylation-Based Biomarkers in Bladder Cancer. Nat. Rev. Urol. 2013, 10, 327-335.
26. Jahr, S.; Hentze, H.; Englisch, S.; Hardt, D.; Fackelmayer, F. O.; Hesch, R. D.; Knippers, R. DNA Fragments in the Blood Plasma of Cancer Patients: Quantitations and Evidence for Their Origin from Apoptotic and Necrotic Cells. Cancer Res. 2001, 61, 1659-1665.
27. Mouliere, F.; Robert, B.; Peyrotte, E. A.; Del Rio, M.; Ychou, M.; Molina, F.; Gongora, C.; Thierry, A. R. High Fragmentation Characterizes Tumour-Derived Circulating DNA. PLoS One 2011, 6, e23418.
28. Murrell, A.; Rakyan, V. K.; Beck, S. From Genome to Epigenome. Hum. Mol. Genet. 2005, 14, R3-R10.
29. Grunau, C.; Renault, E.; Rosenthal, A.; Roizes, G. Methdb; A Public Database for DNA Methylation Data. Nucleic Acids Res. 2001, 29, 270-274.
30. Tian, K.; He, Z. J.; Wang, Y.; Chen, S. J.; Gu, L. Q. Designing a Polycationic Probe for Simultaneous Enrichment and Detection of Micrornas in a Nanopore. ACS Nano 2013, 7, 3962-3969.
31. Kang, I.; Wang, Y.; Reagan, C.; Fu, Y. M.; Wang, M. X.; Gu, L. Q. Designing DNA Interstrand Lock for Locus-Specific Methylation Detection in a Nanopore. Sci. Rep. 2013, 3, 2381.
32. Wang, Y.; Zheng, D. L.; Tan, Q. L.; Wang, M. X.; Gu, L. Q. Nanopore-Based Detection of Circulating Micrornas in Lung Cancer Patients. Nat. Nanotechnol. 2011, 6, 668-674.
33. Shim, J.; Humphreys, G. I.; Venkatesan, B. M.; Munz, J. M.; Zou, X. Q.; Sathe, C.; Schulten, K.; Kosari, F.; Nardulli, A. M.; Vasmatzis, G.; et al. Detection and Quantification of Methylation in DNA Using Solid-State Nanopores. Sci. Rep. 2013, 3, 1389.
34. Shim, J.; Gu, L. Q. Single-Molecule Investigation of G-Quadruplex Using a Nanopore Sensor. Methods 2012, 57, 40-46.
35. Gu, L. Q.; Shim, J. W. Single Molecule Sensing by Nanopores and Nanopore Devices. Analyst 2010, 135, 441-451.
36. Shasha, C.; Henley, R. Y.; Stoloff, D. H.; Rynearson, K. D.; Hermann, T.; Wanunu, M. Nanopore-Based Conformational Analysis of a Viral RNA Drug Target. ACS Nano 2014, 8, 6425-6430.
37. Kurz, V.; Nelson, E. M.; Shim, J.; Timp, G. Direct Visualization of Single-Molecule Translocations through Synthetic Nanopores Comparable in Size to a Molecule. ACS Nano 2013, 7, 4057-4069.
38. Carlsen, A. T.; Zahid, O. K.; Ruzicka, J. A.; Taylor, E. W.; Hall, A. R. Selective Detection and Quantification of Modified DNA with Solid-State Nanopores. Nano Lett. 2014, 14, 5488-5492.
39. Branton, D.; Deamer, D. W.; Marziali, A.; Bayley, H.; Benner, S. A.; Butler, T.; Di Ventra, M.; Garaj, S.; Hibbs, A.; Huang, X. H.; et al. The Potential and Challenges of Nanopore Sequencing. Nat. Biotechnol. 2008, 26, 1146-1153.
40. Venkatesan, B. M.; Bashir, R. Nanopore Sensors for Nucleic Acid Analysis. Nat. Nanotechnol. 2011,6, 615-624.
41. Wanunu, M.; Cohen-Karni, D.; Johnson, R. R.; Fields, L.; Benner, J.; Peterman, N.; Zheng, Y.; Klein, M. L.; Drndic, M. Discrimination of Methylcytosine from Hydroxymethylcytosine in DNA Molecules. J. Am. Chem. Soc. 2011, 133, 486-492.
42. Shim, J.; Rivera, J.; Bashir, R. Electron Beam Induced Local Crystallization of HfO₂ Nanopores for Biosensing Applications. Nanoscale 2013, 5, 10887-10893.
43. Venkatesan, B. M.; Dorvel, B.; Yemenicioglu, S.; Watkins, N.; Petrov, I.; Bashir, R. Highly Sensitive, Mechanically Stable Nanopore Sensors for DNA Analysis. Adv. Mater. 2009, 21, 2771-2776.
44. Jorgensen, H. F.; Adie, K.; Chaubert, P.; Bird, A. P. Engineering a High-Affinity Methyl-Cpg-Binding Protein. Nucleic Acids Res. 2006, 34, e96.
45. Buck-Koehntop, B. A.; Martinez-Yamout, M. A.; Dyson, H. J.; Wright, P. E. Kaiso Uses All Three Zinc Fingers and Adjacent Sequence Motifs for High Affinity Binding to Sequence-Specific and Methyl-Cpg DNA Targets. FEBS Lett. 2012, 586, 734-739.
46. Bowers, P. M.; Schaufler, L. E.; Klevit, R. E. A Folding Transition and Novel Zinc Finger Accessory Domain in the Transcription Factor Adr1. Nat. Struct. Biol. 1999, 6, 478-485.
47. Drew, H. R.; Wing, R. M.; Takano, T.; Broka, C.; Tanaka, S.; Itakura, K.; Dickerson, R. E. Structure of a B-DNADodecamer: Conformation and Dynamics. Proc. Natl. Acad. Sci. U.S.A. 1981, 78, 2179-2183.
48. Ohki, I.; Shimotake, N.; Fujita, N.; Jee, J.; Ikegami, T.; Nakao, M.; Shirakawa, M. Solution Structure of the Methyl-Cpg Binding Domain of Human Mbdl in Complex with Methylated DNA. Cell 2001, 105, 487-497.
49. Cerf, A.; Cipriany, B. R.; Benitez, J. J.; Craighead, H. G. Single DNA Molecule Patterning for High-Throughput Epigenetic Mapping. Anal. Chem. 2011, 83, 8073-8077.
50. Smeets, R. M. M.; Kowalczyk, S. W.; Hall, A. R.; Dekker, N. H.; Dekker, C. Translocation of Reca-Coated Double-Stranded DNA through Solid-State Nanopores. Nano Lett. 2009, 9, 3089-3095.
51. Wanunu, M.; Sutin, J.; McNally, B.; Chow, A.; Meller, A. DNA Translocation Governed by Interactions with Solid-State Nanopores. Biophys. J. 2008, 95, 4716-4725.
52. Zou, H. Z.; Harrington, J.; Rego, R. L.; Ahlquist, D. A. A Novel Method To Capture Methylated Human DNA from Stool: Implications for Colorectal Cancer Screening. Clin. Chem. 2007, 53, 1646-1651.
53. Carlsen, A. T.; Zahid, O. K.; Ruzicka, J.; Taylor, E. W.; Hall, A. R. Interpreting the Conductance Blockades of DNA Translocations through Solid-State Nanopores. ACS Nano 2014, 8, 4754-4760.
54. Ehrlich, M. DNA Hypomethylation in Cancer Cells. Epigenomics 2009, 1, 239-259.
55. Krausz, C.; Sandoval, J.; Sayols, S.; Chianese, C.; Giachini, C.; Heyn, H.; Esteller, M. Novel Insights into DNA Methylation Features in Spermatozoa: Stability and Peculiarities. PLoS One 2012, 7, e44479.
56. Lopes, E. C.; Valls, E.; Figueroa, M. E.; Mazur, A.; Meng, F. G.; Chiosis, G.; Laird, P. W.; Schreiber-Agus, N.; Greally, J. M.; Prokhortchouk, E.; et al. Kaiso Contributes to DNA Methylation-Dependent Silencing of Tumor Suppressor Genes in Colon Cancer Cell Lines. Cancer Res. 2008, 68, 7258-7263.
57. Strichman-Almashanu, L. Z.; Lee, R. S.; Onyango, P. O.; Perlman, E.; Flam, F.; Frieman, M. B.; Feinberg, A. P. A Genome-Wide Screen for Normally Methylated Human Cpg Islands That Can Identify Novel Imprinted Genes. Genome Res. 2002, 12, 543-554.
58. Wanunu, M.; Morrison, W.; Rabin, Y.; Grosberg, A. Y.; Meller, A. Electrostatic Focusing of Unlabelled DNA into Nanoscale Pores Using a Salt Gradient. Nat. Nanotechnol. 2010, 5, 160-165.
59. Ahlquist, D. A.; Zou, H.; Domanico, M.; Mahoney, D. W.; Yab, T. C.; Taylor, W. R.; Butz, M. L.; Thibodeau, S. N.; Rabeneck, L.; Paszat, L. F.; et al. Next-Generation Stool DNA Test Accurately Detects Colorectal Cancer and Large Adenomas. Gastroenterology 2012, 142, 248-256.
60. Kim, Y.; Kim, S. H.; Ferracane, D.; Katzenellenbogen, J. A.; Schroeder, C. M. Specific Labeling of Zinc Finger Proteins Using Noncanonical Amino Acids and Copper-Free Click Chemistry. Bioconjugate Chem. 2012, 23, 1891-1901.

### Example 3

### Integrated Systems for Sample Characterization by Nanopores

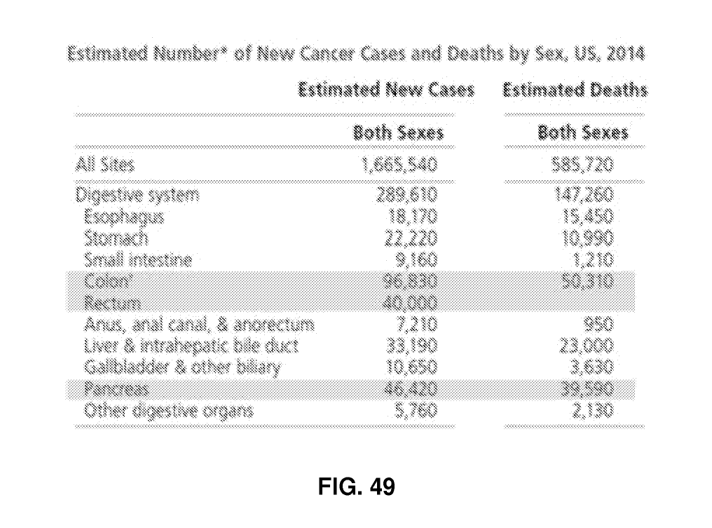

Cancer is one of the leading causes of death in the United States accounting for nearly 1 in every 4 deaths, second only to heart disease. In 2014 alone, over 585,720 Americans were expected to die of cancer, more than 1,600 people a day.¹ According to the American Cancer Society (ACS), about 1.67 million new cancer cases were expected to be diagnosed in the U.S. in 2014 not including the 1 million or so basal and squamous cell skin cancers. As a specific example, colorectal cancer and pancreatic cancer are the top two gastrointestinal cancers estimated of new cancer cases and deaths at both sexes in the US in 2014. See, e.g., FIG. 49. Typically early stages of colorectal and pancreatic cancers do not have symptoms. Conventional screening methods for colorectal cancers are invasive and lack accuracy.³ Due to the tendency of colorectal cancer occurrence to the individuals 50 years and older, colonoscopy screening is not generally given to the patients younger than 50 years, but colorectal cancer rate at young age is increasing. Consequently, timely evaluation of symptoms consistent with colorectal cancer, more patient-friendly and accurate approaches, is essential. At present, there is no reliable method for the early detection of pancreatic cancer, and detection at late-stage presents high mortality. Thus effective early detection methods are greatly needed. In 2014, an important and costly public health issue in the U.S. with combined estimate of colorectal and pancreatic cancers deaths about 90,000 people; reach to 61% death of all digestive system cancer and 15% death of all sites cancer patients. Especially, from 2006 to 2010, the death rate for pancreatic cancer increased by 0.4% per year.¹ To curb the high mortality rates from pancreatic cancer and to provide screening for early-detection, effective and affordable diagnostic method is urgently desired.

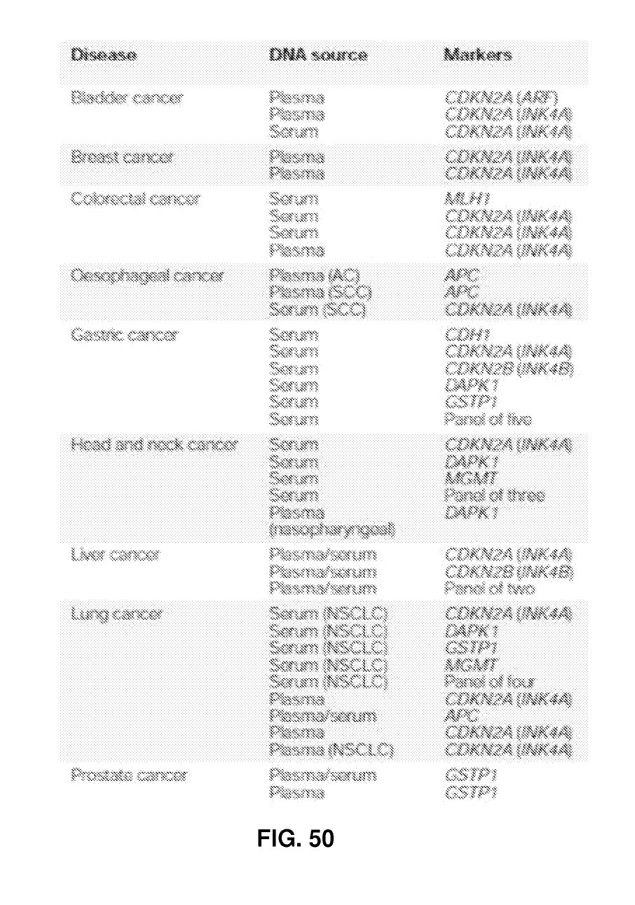

As an epigenetic biomarker for cancer, the methylation profiles in human DNA hold enormous potential and may allow molecular staging, consequently utilizing methylation profiles is a promising approach for early cancer diagnosis, and monitoring progression and recurrence.⁴⁻⁸ Alterations in DNA methylation affect the structure of DNA without modifying the DNA sequence,⁴ and the epigenetic modification of human DNA involves the addition of a methyl group at the carbon-5 position of cytosine (5-methylcytosine) and occurs exclusively at CpG dinucleotides. The bulk of the human genome is depleted of CpG dinucleotides, however, there exist small CpG rich segments termed CpG islands located in the promoter sequences of various genes that are nearly always unmethylated.^{9,10} Hypermethylation of these islands has been associated with transcriptional repression through mechanisms such as the recruitment of methylated CpG binding proteins, histone deacetylation and chromatin remodeling.^{11,12} Aberrant methylation of these gene promoters can point to specific pathways disrupted in every type of cancer and can provide markers for sensitive detection of virtually all tumor types.¹³ In fact, the tumor prevalence of many methylation markers is considerably higher than that of genetic markers.⁹ Interestingly, epigenetic biomarker of methylated DNA (methDNA) can be obtained through noninvasive collection method. FIG. 50 shows a list of cancer type and methDNA that can be obtained from serum and plasma.^{2,14} In addition, screening methylation profile in stool DNA suggests a new paradigm of simple and noninvasive diagnosis for gastrointestinal cancer.¹⁵⁻¹⁷ Methylation is a predictable assay target on gene promoter regions and its occurrence with high frequency in early-stage of neoplasia is very attractive as biomarker for screening.¹⁸ Aberrant methylation pattern on p16, MGMT, MLH1, SFRP2, HIC1 and vimentin genes have been found in stool.¹⁹⁻²⁴ Pancreatic cancer and precursors exfoliate into the local effluent and ultimately stool. Previous studies demonstrated that mutant KRAS in stool reflects the presence both of pancreatic cancer and precancer, thus pancreatic cancer can also be screened noninvasively.²⁵⁻²⁷ The top four markers found in tissue assay of pancreatic cancer; BMP3, NDRG4, EYA4, UCHL1, and mutant KRAS were evaluated in stool sample. Hypermethylation of BMP3 and mutant KRAS has proven useful in predicting the presence of both of pancreatic and precancer.¹⁶ Hence, screening molecule stage via stool sample is an emerging and alternative noninvasive gastrointestinal cancer screening approach. The technological void however, remains in the development of robust and cost-efficient technologies capable of accurately determining the methylation status of panels of genes from minute clinical sample volume. We predict that solid-state nanopores could help bridge this unmet technological need.

Presented herein are exemplary integrated systems useful in reliable assays for diagnosis of disease states, such as a gastrointestinal cancer, incorporating a number of methodology; (1) Ability to extract DNA of interest from patients' stool sample allows the unique ability to interrogate epigenetic biomarker for gastrointestinal cancer; particularly methylation pattern on genomic DNA can provide diagnosis of the gastrointestinal cancer at its early-stage; (2) At the heart of the innovative method is a new detection approach using solid-state nanopore that allows electrical current signature to identify target methylation profiles on short DNA fragments bound with methyl-CpG-binding protein (MBP). This technique has the potential to detect methylation sites on a wide range of genomic dsDNA while circumventing laborious and low throughput PCR using bisulfite conversion of dsDNA; (3) The innovative on-chip nanopore integrated with a microfluidic channel to collect short DNA fragments captured on beads which are driven by magnetic forces to within a 500 μm×500 μm×150 μm chamber to increase the concentration of target sample at small volume above the nanopores, and the nanopore will detect methylation in short DNA fragments via electrical current signature. These approaches in combination result in a non-laborious, low cost, noninvasive and non-colonoscopic diagnostic tools toward gastrointestinal cancer for early-stage detection and prognosis monitoring. This technique is applicable to other cancers using patient samples from serum, urine, saliva, or biopsy for a range of clinical diagnostics.

The methods and integrated nanopore biosensors provide the most significant clinical diagnostic needs of noninvasive, affordable, and patient-friendly disease detection, including, but not limited to, cancer detection of GI cancers. We focus on detection of the methylation profile in short DNA fragments representative of GI cancer, including complete detection of bare DNA and MBP bound methDNA at single-molecule level through nanopore-based sensors. Other MBP binding to a portion of methylation CpG Island is investigated. Nanopores of diameter of 10 nm or less are used, fabricated in homogeneous membrane of SiN and surface chemically functionalized nanopore (SFN). We then demonstrate a diagnostic sensing technology with on-chip nanopore sensor equipped microfluidic system: extracting stool DNA from patients and forming complex with MBP, introducing the extracted DNA in microfluidic channel and concentrating immediately above a nanopore, releasing the DNA and methDNA/MBP complex from beads, and detecting methylation in DNA through solid-state nanopores. We focus on concentrating extracted DNA from buffer samples in a localized location in microfluidic channel; the extracted DNA in low concentration is attached on magnetic beads; the beads are captured in microfluidic device to increase the DNA concentration at the tiny local area, also referred herein as a “first fluid compartment region”, that is fluidically adjacent to a nanopore entrance”.

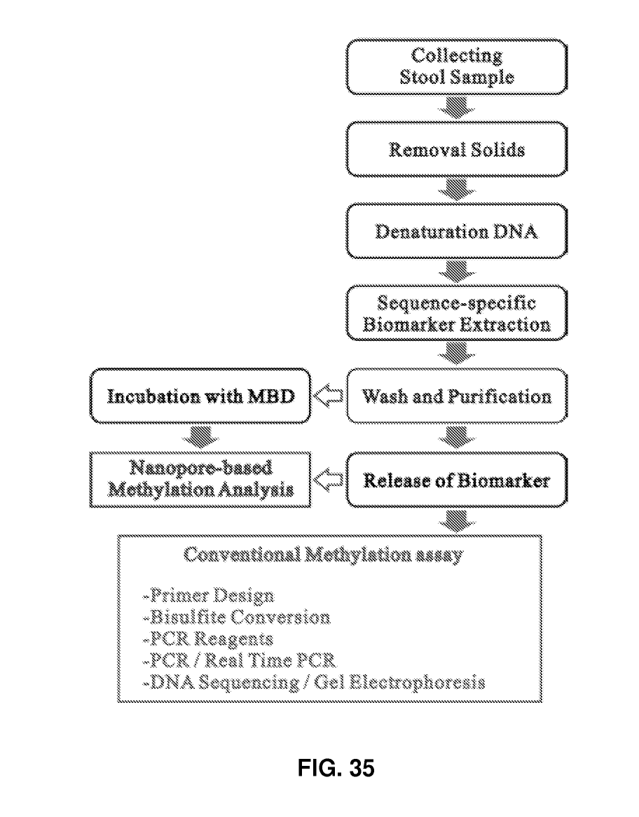

Detection of methylation in short DNA fragments: Current methods for gene based methylation analysis using bisulfite conversion are highly labor intensive, require large sample volumes, suffer from high per run cost and in most cases lack the sensitivity needed to derive useful clinical outcomes.²⁸⁻³¹ In contrast, a nanopore based approach for early cancer detection and prognosis monitoring can deliver the sensitivity and speed needed in extracting useful clinical information, relevant to patient outcome. Nanopores detect and analyze biomolecules at the single-molecule level with high throughput.³² Recent progress in the nanopore research field, solid-state nanopores³³ have shown great promise in healthcare oriented applications such as detecting biomolecules and distinguishing specific molecules in a mixture, leading to the development of diagnostic methods. Nanopore-based sensors have tremendous potential to discover novel methods for detecting disease and saving human life^{33,34} and for developing next generation DNA sequencing tools.³⁵ As an investigation tool, nanopores have shown versatility in label-free DNA/RNA analysis. Nanopore-based sensor is obvious to create innovative healthcare applications. Our approach using nanopore-sensor is well suited for methylation analysis and is preferred over conventional methylation detection strategies due to its ability to (1) detect target molecules at low concentrations from minute sample volumes (2) detect a combination of methylation aberrations across a variety of genes (important in monitoring disease progression and prognosis) (3) detect subtle variations in methylation patterns across alleles that would not be detected using bulk ensemble averaging methods such as PCR and gel-electrophoresis (4) perform rapid methylation analysis (5) reduce cost and simplify steps of experiment and analysis by eliminating cumbersome PCR, DNA sequencing and bisulfite conversion steps (FIG. 35).

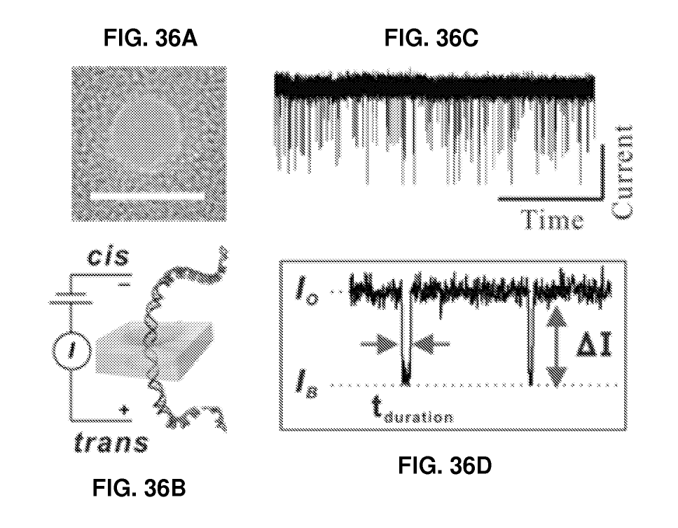

Initial research focuses on the development of robust and versatile nanopores sensor for DNA analysis, and on the investigation of biophysics of single molecules. Solid-state nanopores are nanometer sized apertures formed in thin synthetic dielectric membranes (FIG. 36A). The diameter of a nanopore is fabricated comparable to cross-sectional diameter of a target individual single molecule then inserted into a flow cell containing two chambers filled with conductive electrolyte. Target DNA molecules are next inserted into the cis chamber of the fluidic setup. Two-terminal electrophoresis is used to drive the negatively charged DNA molecule through the nanopore (FIG. 36B), resulting in a transient blockade in the open pore current as seen in FIG. 36C. These electrical signatures are then analyzed, revealing useful information about the translocating molecule (FIG. 36D). These nanopore sensors exhibit excellent mechanical robustness and outstanding electrical performance, allowing them ideal DNA analysis sensor. Single DNA molecule detection was demonstrated through Al₂O₃ involving 5 kbsp dsDNA.^{36,37} HfO₂, a high-k material, nanopores showed mechanical and chemical stability in solution, making them applicable to ionic field effect transistor. Also, HfO₂ nanopores reveal improved local hydrophilicity near nanopore for better detection of single DNA molecules.³⁸ Recently, we reported a new single molecule assay for the detection and quantification of methDNA using solid-state nanopores. MethDNA in complex with a single MBP is detected with significantly discrimination to unmethylated DNA, giving a resolution of a single methylated CpG dinucleotide.³⁹ This nanopore-based methylation sensitive assay circumvents the need for bisulfite conversion, fluorescent labeling, and PCR and could therefore prove very useful in studying the role of epigenetics in human disease. The successful result of detection and quantification based on actual cancer-specific dsDNA emphasizes broad impacts of nanopore analysis on cancer-specific sensor development and cancer-specific methylation pattern on promoter. These studies confirm that it is indeed possible to use nanopores for ultra-sensitive genetic analysis and likely also for epigenetic analysis at low concentration of DNA sample amount.

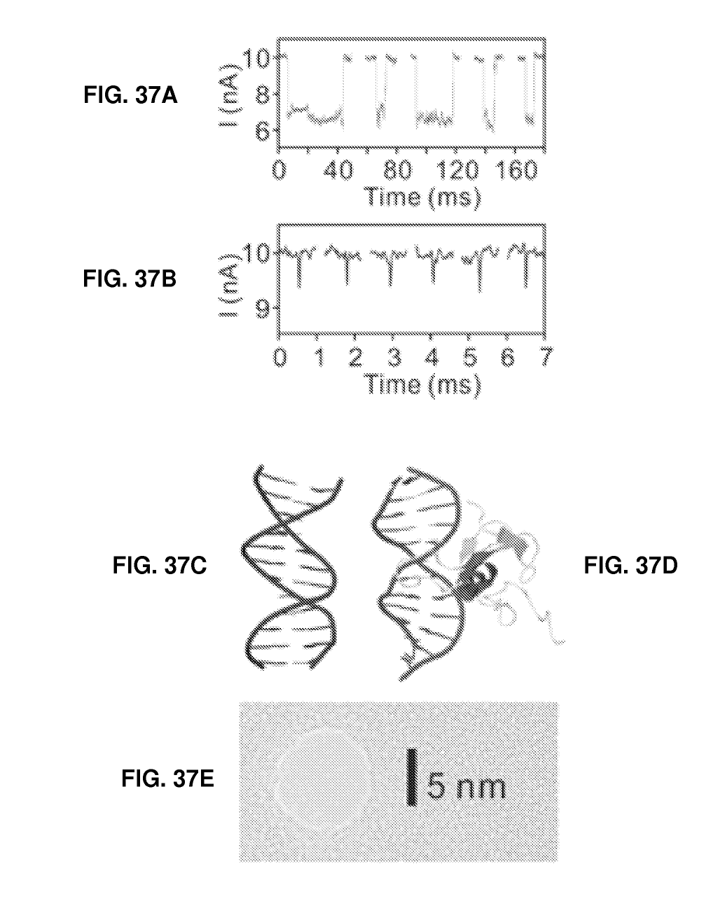

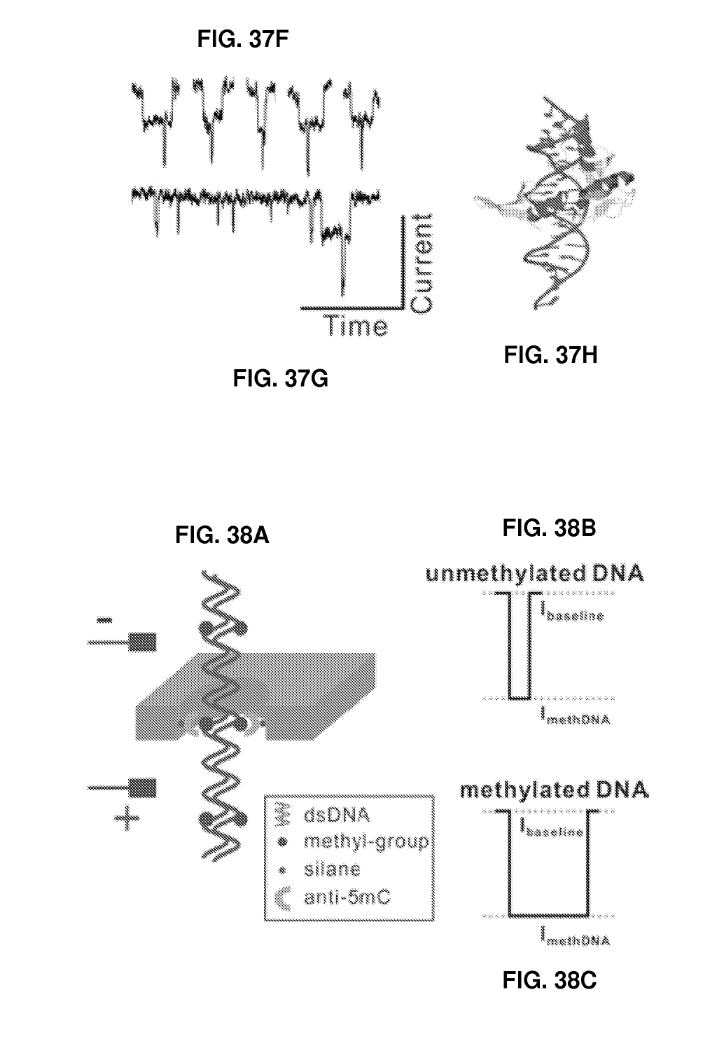

Analysis of short DNA fragments: Most genomic stool DNA will have been digested into short fragments by restriction enzyme before delivered to the nanopore sensor. Due to the short length, swift transport duration of DNA through the nanopore-based sensor will make detection challenging. We synthesize control DNA (IDTDNA, Coralville, Iowa), imitating patients' stool DNA in length and methylation pattern. These control DNA dedicated for the control nanopore experiment, determining the optimal specification of the nanopore in terms of diameter and thickness. Nanopores in various diameters are fabricated in 10 nm-thick SiN membrane, widely used in nanopore research, and detected 90 bp DNA using 8 nm nanopore (FIG. 37A-37H). The short nucleotides have been successfully detected in other studies using 4 nm or smaller nanopore.^{40,41} However, the target genes from stool samples could be from 30-90 bp range.⁴² The passage of shorter DNA fragment through the nanopore will produce faster transport duration with slight deviation in the baseline current. 30, 45, and 60 bp long dsDNA are commercially available and may be used with different nanopore-membranes (Si₃N₄, Al₂O₃, HfO₂) to slow down the transport of DNA through the nanopore, to allow for more robust discrimination of short DNA fragments.

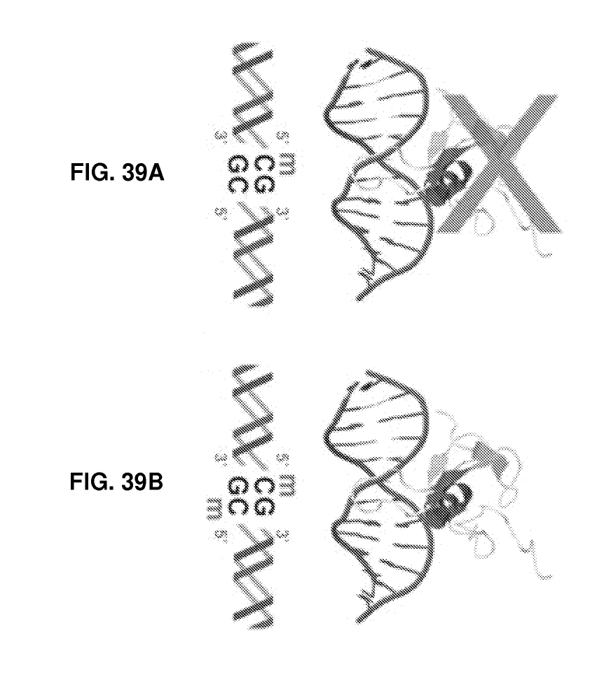

Analysis of MBP bound methDNA: Methylation in short DNA fragments will be detected with binding of MBPs. The passage of a MBP bound methDNA fragment through the nanopore will result in significantly different current signature from the passage of a bare DNA fragment. As the drop in pore current is attributed to the cross section of the translocating molecule, deeper current blockade are observed when the large, bound protein traverses the nanopore. Two types of MBP are used: MBD1x and KZF. These MBPs are engineered to contain only key element, required for binding to methylated CpG on DNA; MBD1x is key methyl-CpG-binding domain of Methyl-CpG-Binding Domain Protein (MBD);⁴³ KZF is key domain binding to DNA of Kaiso Zinc Finger Protein.^{44,45} Consequently, these MBPs in compact size spanning to reduced number of base-pairs compared to its original protein form, therefore it could give more precise resolution of methylated CpG sites. Most of all, the compact dimension of these MBPs contributes to reduced dimension of the nanopores; making nanopore-based detection feasible for bare DNA. Otherwise, it would be more challenging to detect unlabeled short unmethylated DNA fragments using relatively large nanopore dimension of which would be required if using original form of MBPs. MBD1x spans 56 bps on DNA upon binding and molecular weight of 16.3 kDa,³⁹ and KZF wraps around DNA, contacting 56 bps in total.⁴⁴ Crystal structures of two MBPs on methylated DNA are shown in FIG. 37D for MBD1x and FIG. 37F for KZF.^{44,46} MBD1x protein will be introduced to methDNA fragments and incubated in room temperature for 15 minutes to form complex structure of methDNA/MBD1x. We detect the MBD1x bound DNA fragments and discriminate methDNA from unmethylated DNA through sub 10 nm nanopore. We expect two distinct current levels to be observed, the first corresponding to transports of DNA that do contain bound protein (FIG. 38A), and the second corresponding to transports of DNA that do not contain bound protein (FIG. 38B). Also, transport duration of MBP bound DNA is expected to be prolonged compared to bare DNA without MBP. The extended transport duration is attributed to the net positive charge of MBD1x at pH 7.6 of nanopore experimental solution, helping reduce the velocity of complex transport through the SiN nanopore, net negative charge.³⁹ In addition, we utilize KZF protein to recognize two adjacent methylated CpGs on dsDNA. We synthesize 30, 45, 60, and 90 bp-long dsDNA equipped with symmetric mCpGmCpG at the middle of the sequence. KZF protein is mixed with the DNA and incubated at room temperature for 15 minutes. Mixture of KZF bound methDNA fragments and unmethylated DNA fragments are delivered to the nanopore-based sensor. The nanopore-based detection of KZF bound DNA fragments containing symmetric two adjacent methylated CpG are shown in FIG. 39B, and mixture of unmethylated DNA and KZF bound methDNA are shown in FIG. 39C.

Advanced analysis of methDNA using SFN: The detection of unlabeled methDNA (no bound MBP) using surface-functionalized nanopores (SFN) with anti-5-Methylcytosine (anti-5mC) antibodies is also attractive. The specificity and sensitivity of solid-state nanopore to methylation in DNA fragments can be greatly enhanced through surface chemical modification, using commercially available anti-5mC antibody. Translocation of unlabeled methDNA fragments through this SFN will result in highly specific anti-5mC antibody/methylation interactions that are expected to result in prolonged transport duration (FIG. 38). Binding events during translocation are not expected to be permanent due to the short interaction times allowed (less than a ms) for a translocating molecule. Note, the translocation velocity of bare DNA through 10 nm SiN nanopore is ˜1.4 nucleotide/μs. This technique can permit real time comparisons between unmethylated and fully methDNA samples for both in single strand and double strand and is likely capable of detecting densely methylated regions without the spatial limitations (5-6 bp) associated with MBP binding. The functionalization protocol requires the attachment of anti-5mC antibodies (Zymo Research) to the pore surface. Anti-5mC has been chosen as it is monoclonal and can differentiate between methylated and unmethylated cytosines in DNA. This antibody has been successfully used in Methylated DNA Immunoprecipitation assays and is ideal for our application. Longer translocation times are expected for methylated fragments relative to unmethylated fragments through an anti-5mC coated nanopore due to specific interactions between methyl-cytosines and immobilized proteins as seen in FIG. 38. The translocation duration should be a function of the overall level of methylation of the target strand. This SFN has been used for sensitive and selective detection of single nucleotide polymorphisms, associated with various cancers, breast^{47,48} and lung⁴⁹⁻⁵¹ cancers.

We target detecting MBP bound methDNA (10% methylation) and discrimination from unmethylated DNA with statistically significant difference in current blockage amplitude and duration. As described herein, we roughly quantify the methylation sites on the 827 bp DNA with careful analysis of transport duration.³⁹ Similarly, we quantify the methylation sites on short DNA fragment with varying transport duration as well. As used herein, a short DNA fragment may refer to an oligonucleotide of less than 100 bp. In addition, KZF can detect single site of symmetric two continuous mCpGmCpG on 90 bp DNA. Consequently, KZF can be very useful to detect fragmented methylated CpG Island with less concentration of protein. Furthermore, SFN detect methylation with interaction between two chemicals without using MBP bound on DNA, thus this approach can be applied to single-stranded DNA as well. Our group has extensive experience in fabrication of nanopore as small as 1 nm in diameter so we expect to investigate methylated single-strand DNA as well.

The 30 bp long genes present challenges, but proper choice of dielectric and surface functionalization can slow the molecule enough to be detected via a 10 nm-thick Si₃N₄ member. Due to the short length of DNA fragments, methylation mapping on DNA would not be achieved through 10 nm-thick homogeneous membrane nanopore. While profiling is not needed for our specific application and information of hyper versus hypomethylation is extremely useful in itself, we can explore the use of a graphene layer embedded for sensitive discrimination. A single layer of graphene sheet as sensing electrode sandwiched between two dielectric layers from can possess the ability to sense a translocating molecule locally at the middle of membrane with 0.34 nm resolution.⁵² See also, U.S. Pub. No. 2014/0174927. Furthermore, a potential voltage can be applied through the graphene electrode to trap a MBP-bound region, because MBP is positively charged in solution due to the isoelectric point.³⁹ This graphene could be used to study electrochemical exchange at an individual graphene edge and modulation of ionic current at the middle in the nanopore.⁵²

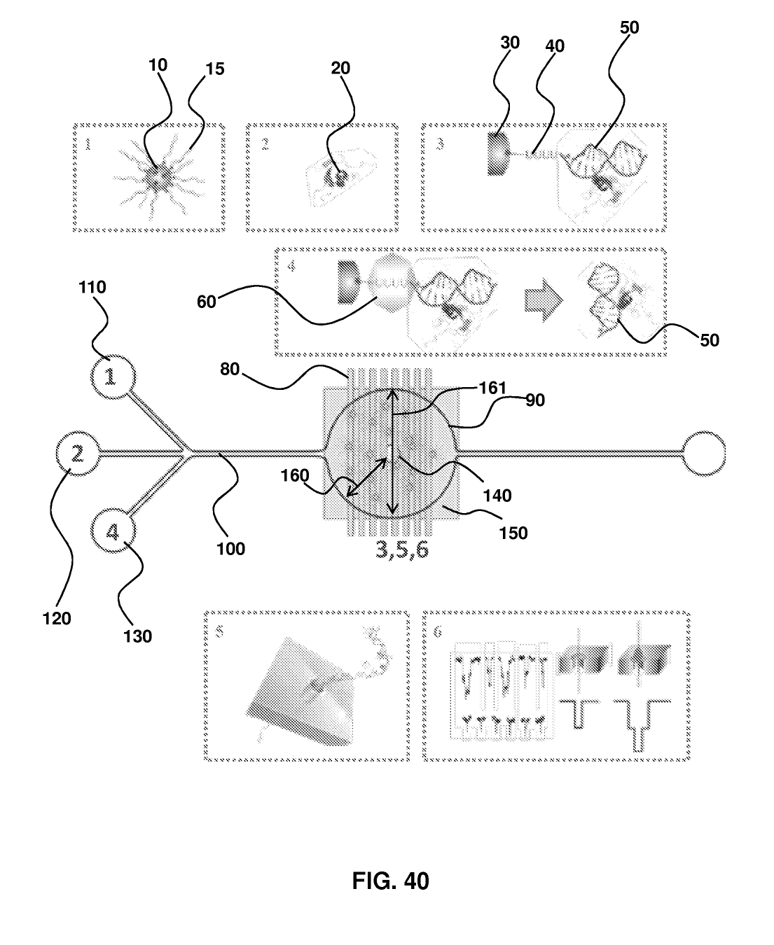

Integrated diagnostic method using methylation profile detection: We demonstrate the feasibility of a complete diagnostic methods with control DNA from control samples (but designed toward later clinical samples of gastrointestinal cancer). Due to the ultra-low concentration of patients' stool DNA sample and to avoid laborious and low throughput of bisulfite treats and methyl specific PCR reaction, it is necessary to concentrate target DNA locally near the sensing element, i.e. the nanopore for integrated diagnosis. To fulfill this demand, we utilize a microfluidic system equipped with magnetic force driven beads as a collection technique. We will develop a nanopore-based on-chip sensor integrated with microfluidic system; introducing stool DNA on beads in microfluidic system, collecting beads immediately above the nanopore, release DNA and complex of DNA/protein, and detect the biomarkers using solid-state nanopore. Consequently, we make complex DNA/MBP on the beads and release the targets at very close distance from the nanopore. The concentration of DNA/beads can be performed via magnetic fields. The target DNA is extracted through methylated single stranded probe complementary to the target. The probe can be amino conjugated to the carboxylic acid-coated beads, bound to methyl-binding protein and equipped with releasable chemical linker between the bead and probe molecule. The probe is designed to have methylation on all CpG sites and four uracils in between terminal amino group and probe which that act as the releasable linker. FIG. 40 shows the overall scheme.

Briefly a capture element 10 illustrated as a magnetic bead with polynucleotide of interest 15 attached thereto (step 1). A biomarker 20 (step 2) may be provided that specifically binds to polynucleotide of interest exhibiting a biomolecular parameter. Step 3 is a close-up view of one DNA:biomarker complex 50, connected to bead surface 30 via cleavable linker 40. In this example, biomarker 20 is a MBD1x protein that binds methylated cytonsine. In step 4, a release element 60 selectively cleaves at the cleavable linker 40 to release the DNA:biomarker complex 50. The middle panel is a schematic illustration as to how steps 1-4 may be implemented within an integrated diagnostic system. Bead 10 with polynucleotide 15 obtained from a sample connected thereto via cleavable linker 40 may be provided to a microfluidic passage 100, which, in turn, fluidically transports the polynucleotide from the sample with the bead to a first fluid compartment region 90. Magnet 80 may capture the polynucleotide of interest in a region that is adjacent to the nanopore 140, specifically part of top fluid compartment formed in part by dielectric membrane top surface 150. To facilitate capture, the microfluidic passage 100 may have a cross-sectional area that is less than the cross-sectional area of the first fluid compartment region 90, that can substantially expand around the nanopassage pore by a separation distance indicated by arrow 160, such as a distance of between about 100 μm and 1000 m. In contrast, the microfluidic channel may have a characteristic cross-section distance of between 1 μm and 1000 μm. The first fluid compartment region may have a maximum cross-sectional area to flow, as indicated by arrow 161. This may be expressed as a ratio of cross-sectional area of flow at 161 to microfluidic passage 100 cross-sectional area to flow, that is greater than or equal to 10, 50, 100, or 500. In this manner, fluid velocity slows over the nanopore region, encouraging both settling of beads, and increase capture time via the capture element component 80, exemplified as magnetic beads 10 and magnetic elements 80. Other forces, of course may be used, including electrokinetic paired with compatible polynucleotides and/or beads. The various fluidic components are added to the microfluidic channel, as indicated by 110 (sample), 120 (biomarker), and 130 (release element). Accordingly, any of the methods and systems may further comprise the step decreasing polynucleotide of interest flow velocity in a region adjacent to a nanopore entrance, thereby increasing the time for capture in a desired region of the first fluid compartment, and improvising distribution relative to the nanopore entrance to provide the functional benefit of increased sensitivity, signal to noise, and overall reliability and robustness of the method.

Turning to the solid state nanopore and related elements, FIG. 1A provides further clarification. Solid state nanopore 140 traverses dielectric membrane 150, having a top surface 152 and a bottom surface 154 with nanopore having entrance 160 and an exit 170. A power supply 180 is electrically connected to first fluid compartment 182 and second fluid compartment 184 to provide an electric potential difference (indicated by − cis and + trans) to force polynucleotide 15 through the nanopore. Detector, 181, which may be integrated with power supply 180, monitors passage parameter output 185, illustrated in FIG. 1C as currents Iₒ, I_{b} and resultant blockade current ΔI and transit duration t_{duration}.

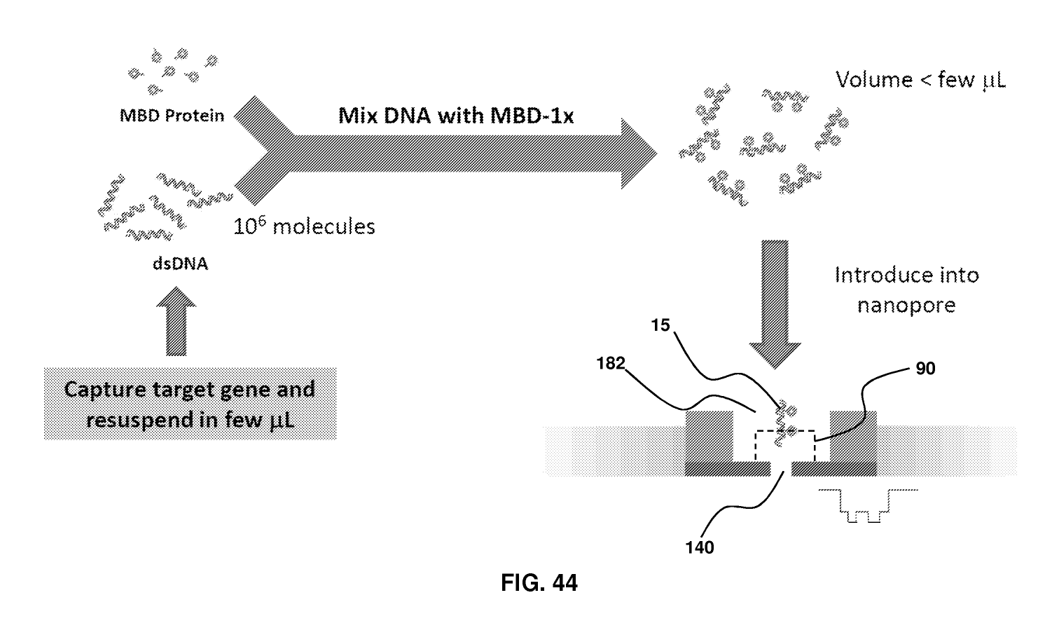

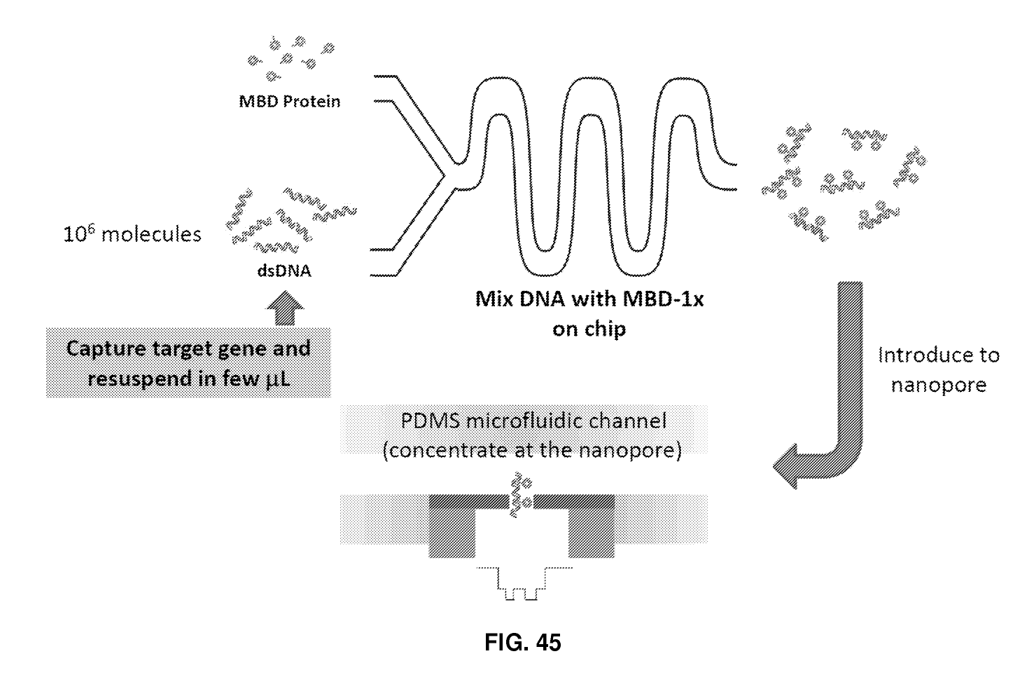

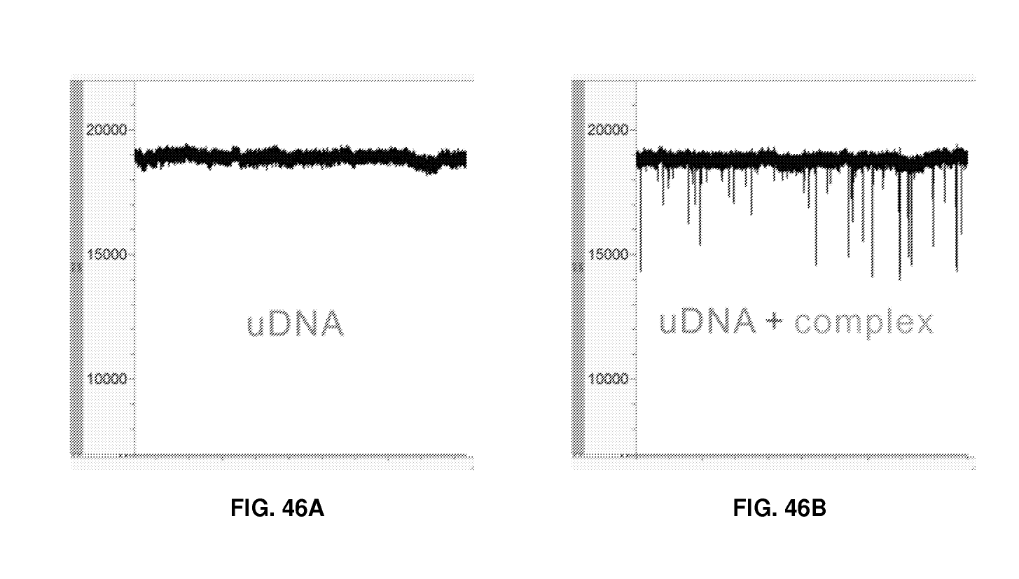

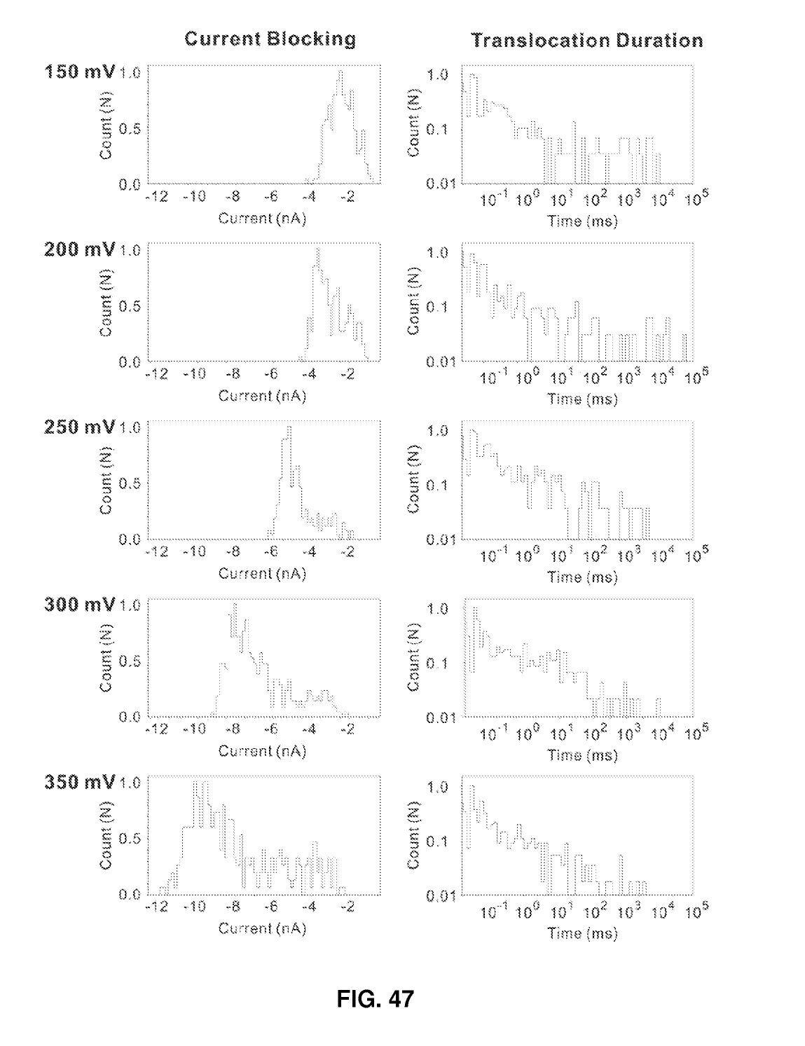

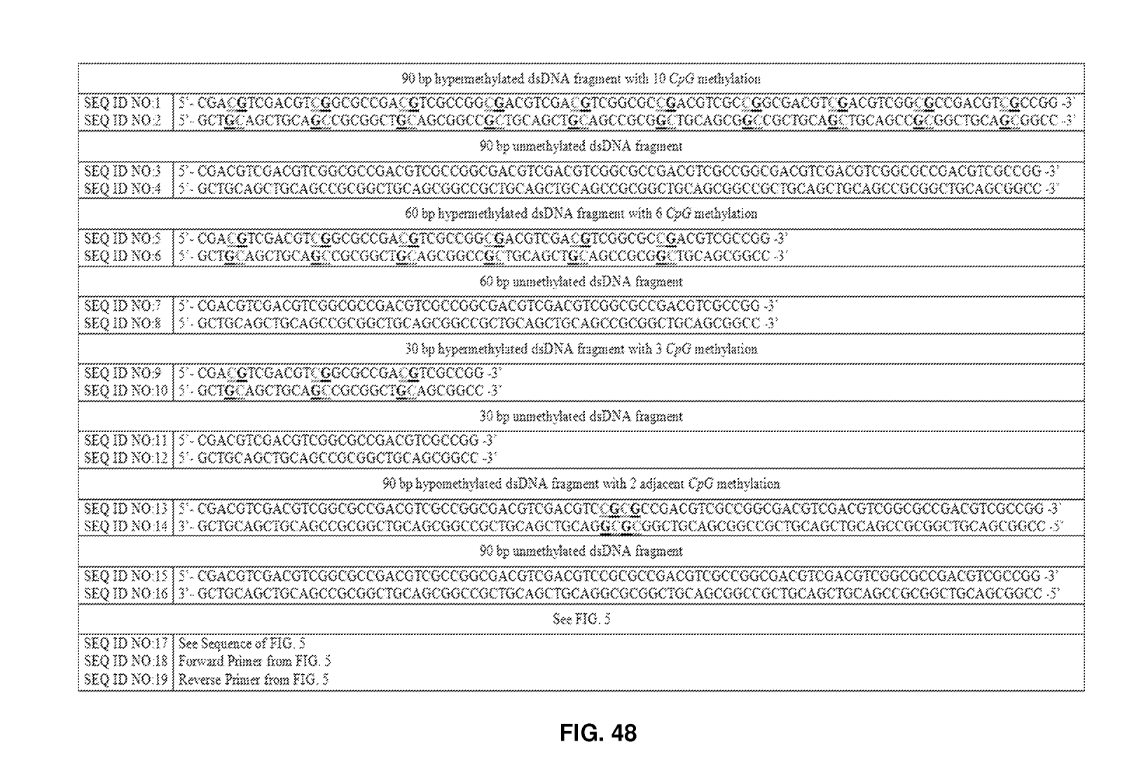

FIG. 44 further illustrates complex 50 traversing nanopore 140 from first fluid compartment 182. As discussed, provided herein are various means for concentrating DNA in a first fluid compartment region 90 that may be, as desired, even closer to the the nanopore than edges of compartment 182.

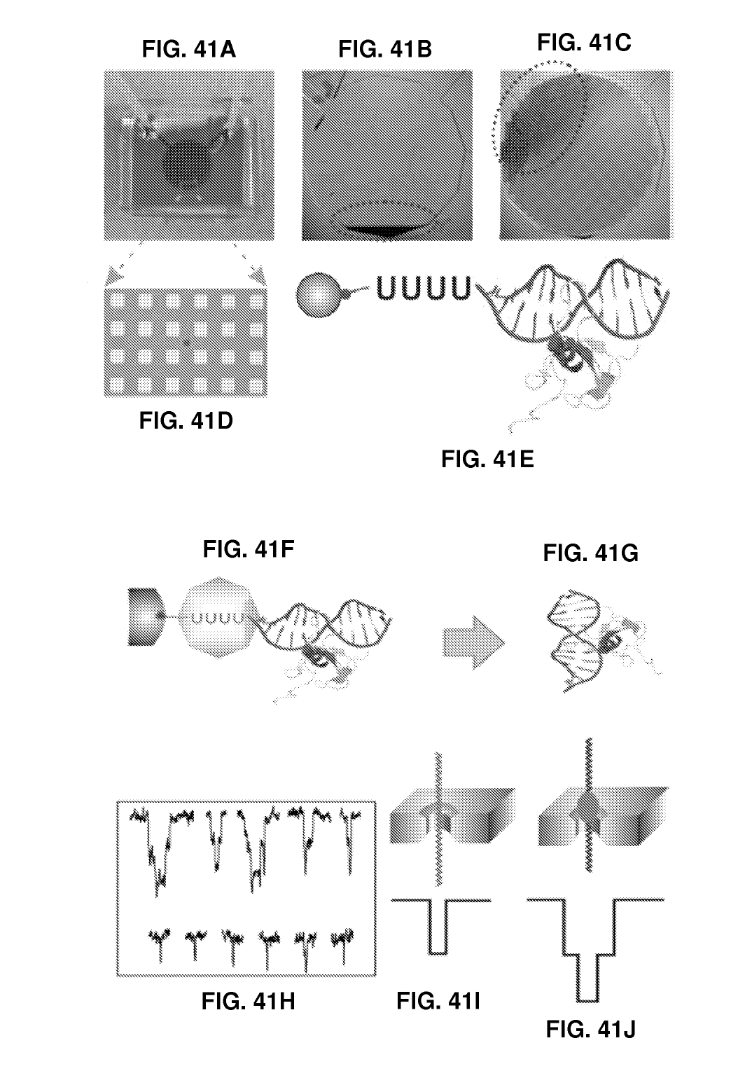

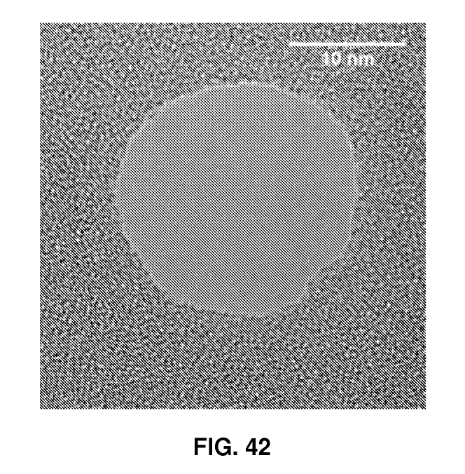

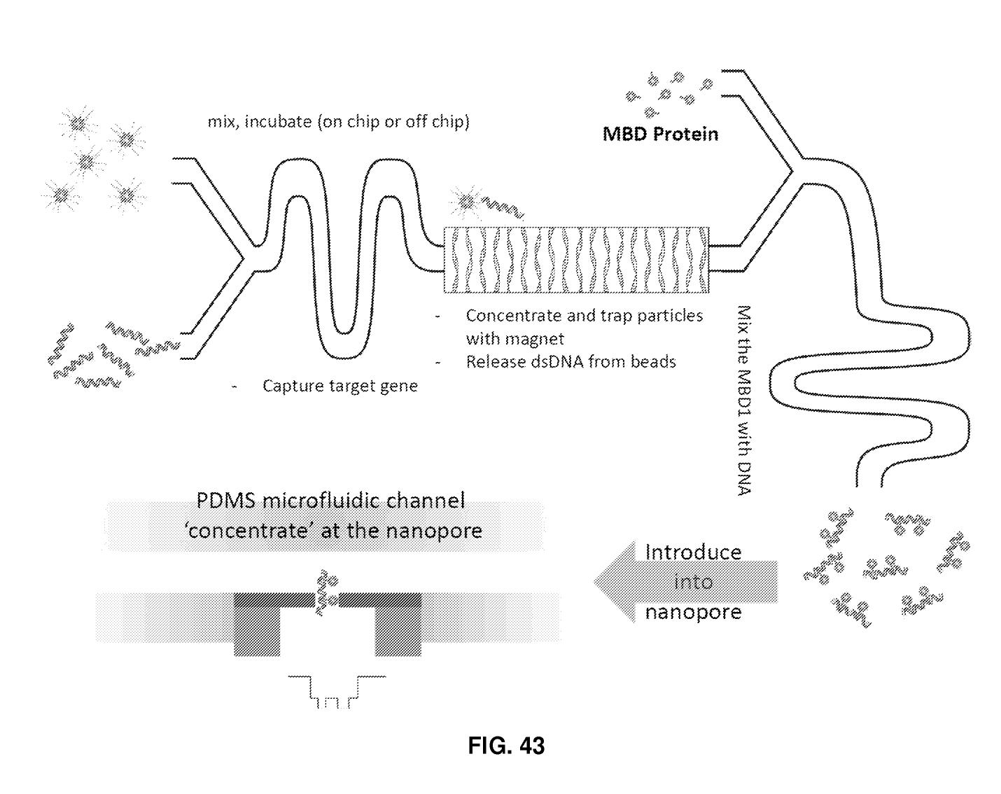

We have successfully extracted genomic methylated stool DNA through sequence-specific purification. Stool DNA is isolated from solids and clarified. To capture the target DNA, an amount of 150 μl of carboxylic acid-coated beads with amino conjugated oligonucleotides complementary to target sequence (IDTDNA) is added and mixed to allow hybridization at room temperature. Supernatant is removed when sample tubes were placed on magnetic beads, then washed in MOPs buffer (10 mM MOPS, 150 mM NaCl, pH 7.5) to remove remaining inhibitors. Finally, heated tRNA elution buffer was added and the beads removed with centrifugation to collect target DNA. After capturing, target DNA was bisulfite treated and amplified using methylation-specific PCR (MSP) reactions.¹⁶ Most commonly used gold standard assay technique so far for DNA methylation assay requires bisulfite conversion and amplification due to low-concentration of DNA analytes obtained from patients' sample. Consequently, alternative method to increase DNA concentration at the sensing point is demanded for amplification-free high throughput DNA assay. To achieve reliable and fast detection of DNA using a nanopore-based sensor, high concentration of DNA close to the nanopore sensor is required. We will use carboxylic acid-coated magnetic beads to capture and deliver DNA to the specific local volume in on-chip nanopore sensor. Magnetic beads are easily pulled to the permanent magnet and carboxylic acid-coated surface can capture DNA (FIGS. 41A-41B). In addition, magnetic-force driven beads collection can avoid thermal heating problem that can occur with a dielectrictrophoresis (DEP) method, denaturing DNA and generating unexpected flow stream vortex lifting beads highly. A commercially available strong neodymium magnet may be used. In addition, we pattern micromagnet on the membrane of nanopore-based sensor to accommodate additional magnetic force, increasing beads capture efficiency and distributing beads uniformly near the nanopore sensor.

Extracting DNA fragments in buffer solution. The methods and systems described herein provide for an improved extracting method that can handle multiple functions: capturing DNA fragments, allowing biomarker (such as MBP) to selectively bind the DNA (for MBP: only on symmetrically methylated CpG dinucleotides), and releasing DNA from magnetic beads. The methylated DNA fragments are extracted from buffer solution in sequence-specific way using complementary probe. The probe will be designed to be complementary to the target methDNA fragments and amino conjugated to the carboxylic acid-coated beads. Beads will be introduced to the buffer solution containing target and extract target DNA via hybridization with the probe at room temperature. On the probe, all CpG sites will be methylated, and, upon hybridization between complementary probe and target sequence DNA, only the dsDNA paired with methylated target DNA would form symmetric methylation on CpG dinucleotides, which MBP can bind to. We have specially engineered and purified in-vitro MBP and shown that MBD1x binds specifically and exclusively to a single symmetric methylated CpG dinucleotides.³⁹ Consequently, although the fully methylated complementary hybridized with unmethylated target DNA, the hemi-methylation pattern would not allow MBD1x to bind on it. We also have another protein as described earlier, the Kaiso zinc finger (KZF) protein that binds specifically to adjacent two methylated CpG (MpGMpG) site, a pattern that is very common in fragmented methylated CpG island. This KZF is very useful to make a complex with aberrant methylation in CpG islands, which is known to be related with cancer occurrence. As continuous methylation in a row on dsDNA is ubiquitous in CpG island, usage of KZF protein would be suitable to detect cancer gene marker consisted of unspecific methylation pattern in CpG island. We will also examine the binding affinity of KZF to hemi-methylated DNA and single symmetric methylated CpG dinucleotides. It is known that KZF recognizes the asymmetric methylation on single strand, and also recognizes single symmetric methylated CpG dinucleotides with slightly less affinity.⁴⁴ The binding affinity of KZF to hemi-methylated DNA is very useful to extract methylated DNA using unmethylated complementary probe.

Concentrating DNA on-chip microfluidic channel using magnet: We concentrate DNA using magnetic force driven beads collection method. Beads are introduced into a microfluidic flow passing the top of nanopore and collected from the microfluidic flow using a permanent magnet. Magnetic beads must overcome inertial force which follows flow stream line to turn their movement toward the permanent magnet so the average flow velocity should be as small as possible. In order to decrease the flow velocity near the nanopore, 5 mm in diameter flow reservoir was designed. With 5 ul/min flow through the microfluidic flow shown in FIG. 41A, over 89% beads are collected at the desired small volume shown in FIG. 41B. We also patterned micro-fabricated nickel patterns that act as magnets in the microfluidic channel to slow down and distribute the beads more uniformly as shown in FIG. 41C. The Ni patterned magnets amplify the magnetic force in microfluidic channel and beads will be attracted to the magnets, consequently beads will be more uniformly distributed over the magnets. We propose to use these patterned Ni layers as magnetics within the microfluidic channel to collect beads to within a tiny volume and distribute these collected beads near the nanopore (shown in FIG. 41D). In case of no micromagnets and only an external magnet, we will use the topology in FIG. 41B and place the pore where the beads are being collected. In case of the patterned magnet layout in FIG. 41C, the pore is placed in the middle of the circular microfluidic channel. We compare these two cases and compare the captured efficiency and the efficiency of the UUUU cleavage to release the DNA from the bead.

Releasing DNA and methDNA/MBP from the beads: The probe is designed to have four uracils between the amino conjugation terminal and complementary sequence. The four uracils in either form of ssDNA and dsDNA can be recognized and digested by Uracil-DNA glycosylase (UDG) restriction enzyme. Thus these four uracils form the cutting point of DNA from beads. We cut at this site with UDG and release the DNA and MBP bound methDNA from the beads. When the bead collection is saturated, we introduce UDG to the microfluidic flow. Since the beads are uniformly distributed along the rim of micromagnet for the case of FIG. 41B, UDG can readily access the 4Us. We incubate the UDG and beads in room temperature for extended time to allow the UDG to cut as many four uracils as possible. Thus, dsDNA and complex of methyDNA/MBP detach from the beads. After an appropriate incubation time, the voltage across the nanopore is applied to detect the detached DNA and MBP bound methDNA that traverses the nanopore. There is unmethylated DNA with no MBP bound on it, and MBP bound meth DNA. Nanopore-based sensor detect both molecules (FIGS. 41H-41J).

The protocols for the capture of DNA molecules and the integration with the nanopore sensors are optimized. Control experiments of amine conjugation of complementary to beads are performed. We will mix fluorescently labeled complementary with beads and optically monitor the flow of beads with fluorescence microscopy. With this fluorescently complementary, we confirm the conjugation of complementary to beads, beads capturing on micromagnet, and detachment of complementary from beads after adding UDG.

After releasing DNA and complex from the beads, UDG remains near the nanopore and could interfere with the electrical current measuring by touching or blocking the nanopore. However, the isoelectric point of UDG is 9.0 and our nanopore sensing performs in salt solution titrated pH in range of 7.6 and 8.0.⁵³ Thus the protein is positively charged due to the isoelectric and will be pushed away from the nanopore at positively applied potential voltage. In addition to beads collection to increase local DNA concentration, we also can use DNA attraction using different salt ingredients at two chambers of nanopore setup. Previous salt gradient study reported up to 30-fold enhanced detection by providing higher salt gradient to trans side when molecule was added to cis side. Our preliminary typical patient's stool DNA collection can obtain 100,000 molecules in 60 μl with the plus margin to the 200,000 molecules in 60 μl. Wanunu et al. demonstrated successful detection of 1000 events in 15 minutes using DNA concentration of 1,000,000 in 10 μl.⁵⁴ The DNA concentration may increase up to 500-fold near the nanopore area. Thus, increased concentration would be 833,000 molecules in 10 μl. If we use salt gradient in our on-chip, we will have 250,000,000 molecules in 10 μl.

Nanopore-based methylation detection can be extended to include the analysis of clinical stool DNA, specifically the detection of aberrant methylation patterns in stool DNA isolated from stool sample of gastrointestinal cancer patients. Aberrant methylation of the promoter sequences of various genes has been implicated in cancers and easily obtainable noninvasively from patients' body fluid. Nanopore-based gene based methylation detection for small volume can satisfy this important clinical need. The application of this nanopore-based screening of epigenetic cancer is broad and pervasive in providing simple gene based methylation detection for cancer diagnostics and prognostics.

### REFERENCES FOR EXAMPLE 3

1 American Cancer Society. Cancer Facts & Figures 2014. Atlanta: American Cancer Society; 2014.
2 Laird, P. W. The power and the promise of DNA methylation markers. Nature reviews. Cancer 3, 253-266, doi:10.1038/nrc1045 (2003).
3 Osborn, N. K. & Ahlquist, D. A. Stool screening for colorectal cancer: molecular approaches. Gastroenterology 128, 192-206 (2005).
4 Strathdee, G. & Brown, R. Aberrant DNA methylation in cancer: potential clinical interventions. Expert Reviews in Molecular Medicine 4, 1-17 (2002).
Chan, A. O.-O. et al. CpG Island Methylation in Aberrant Crypt Foci of the Colorectum. American Journal of Pathology 160, 1823-1830 (2002).
6 Gallagher, W. M. et al. Multiple markers for melanoma progression regulated by DNA methylation: insights from transcriptomic studies. Carcinogenesis 26, 1856-1867 (2005).
7 Graff, J. R., Gabrielson, E., Fujii, H., Baylin, S. B. & Herman, J. G. Methylation Patterns of the E-cadherin 5â [symbol] ² CpG Island Are Unstable and Reflect the Dynamic, Heterogeneous Loss of E-cadherin Expression during Metastatic Progression. Journal of Biological Chemistry 275, 2727-2732 (2000).
8 Muller, H. M., Fiegl, H., Widschwendter, A. & Widschwendter, M. Prognostic DNA methylation marker in serum of cancer patients. Ann NY Acad Sci 1022, 44-49, doi:1 0.1196/annals.1318.008 (2004).
9 Laird, P. W. The power and the promise of DNA methylation markers. Nature Reviews Cancer 3, 253-266, doi: Doi 10.1038/Nrc1045 (2003).
10 Laird, P. W. & Jaenisch, R. THE ROLE OF DNA METHYLATION IN CANCER GENETICS AND EPIGENETICS. Annual Review of Genetics 30, 441-464, doi: doi:10.1146/annurev.genet.30.1.441 (1996).
11 Robertson, K. D. DNA methylation and human disease. Nature Reviews Genetics 6, 597-610, doi: Doi 10.1038/Nrg1655 (2005).
12 Rountree, M. R., Bachman, K. E., Herman, J. G. & Baylin, S. B. DNA methylation, chromatin inheritance, and cancer. Oncogene 20, 3156-3165 (2001).
13 Esteller, M., Corn, P. G., Baylin, S. B. & Herman, J. G. A Gene Hypermethylation Profile of Human Cancer. Cancer Research 61, 3225-3229 (2001).
14 Summers, T. et al. Serum-based DNA methylation biomarkers in colorectal cancer: potential for screening and early detection. Journal of Cancer 4, 210-216, doi:10.7150/jca.5839 (2013).
15 Zou, H., Harrington, J., Rego, R. L. & Ahlquist, D. A. A novel method to capture methylated human DNA from stool: implications for colorectal cancer screening. Clinical chemistry 53, 1646-1651, doi:10.1373/clinchem.2007.086223 (2007).
16 Kisiel, J. B. et al. Stool DNA testing for the detection of pancreatic cancer: assessment of methylation marker candidates. Cancer 118, 2623-2631, doi:10.1002/cncr.26558 (2012).
17 Imperiale, T. F. et al. Multitarget stool DNA testing for colorectal-cancer screening. The New England journal of medicine 370, 1287-1297, doi:10.1056/NEJMoa1311194 (2014).
18 Zou, H. et al. Frequent methylation of eyes absent 4 gene in Barrett's esophagus and esophageal adenocarcinoma. Cancer epidemiology, biomarkers & prevention: a publication of the American Association for Cancer Research, cosponsored by the American Society of Preventive Oncology 14, 830-834, doi:10.1158/1055-9965.EPI-04-0506 (2005).
19 Chen, W. D. et al. Detection in fecal DNA of colon cancer-specific methylation of the nonexpressed vimentin gene. Journal of the National Cancer Institute 97, 1124-1132, doi:10.1093/jnci/dji204 (2005).
20 Petko, Z. et al. Aberrantly methylated CDKN2A, MGMT, and MLH1 in colon polyps and in fecal DNA from patients with colorectal polyps. Clinical cancer research: an official journal of the American Association for Cancer Research 11, 1203-1209 (2005).
21 Lenhard, K. et al. Analysis of promoter methylation in stool: a novel method for the detection of colorectal cancer. Clinical gastroenterology and hepatology: the official clinical practice journal of the American Gastroenterological Association 3, 142-149 (2005).
22 Leung, W. K. et al. Detection of epigenetic changes in fecal DNA as a molecular screening test for colorectal cancer: a feasibility study. Clinical chemistry 50, 2179-2182, doi:10.1373/clinchem.2004.039305 (2004).
23 Belshaw, N. J. et al. Use of DNA from human stools to detect aberrant CpG island methylation of genes implicated in colorectal cancer. Cancer epidemiology, biomarkers & prevention: a publication of the American Association for Cancer Research, cosponsored by the American Society of Preventive Oncology 13, 1495-1501 (2004).
24 Muller, H. M. et al. Methylation changes in faecal DNA: a marker for colorectal cancer screening? Lancet 363, 1283-1285, doi:10.1016/S0140-6736(04)16002-9 (2004).
25 Zou, H. et al. High detection rates of colorectal neoplasia by stool DNA testing with a novel digital melt curve assay. Gastroenterology 136, 459-470, doi:10.1053/j.gastro.2008.10.023 (2009).
26 Caldas, C. et al. Detection of K-ras mutations in the stool of patients with pancreatic adenocarcinoma and pancreatic ductal hyperplasia. Cancer Res 54, 3568-3573 (1994).
27 Berndt, C. et al. K-ras mutations in stools and tissue samples from patients with malignant and nonmalignant pancreatic diseases. Clinical chemistry 44, 2103-2107 (1998).
28 Eads, C. A. et al. MethyLight: a high-throughput assay to measure DNA methylation. Nucleic Acids Research 28, e32 (2000).
29 Trinh, B. N., Long, T. I. & Laird, P. W. DNA Methylation Analysis by MethyLight Technology. Methods 25, 456-462 (2001).
30 Coleman, W. B. & Rivenbark, A. G. Quantitative DNA methylation analysis—The promise of high-throughput epigenomic diagnostic testing in human neoplastic disease. Journal of Molecular Diagnostics 8, 152-156, doi: DOI 10.2353/jmoldx.2006.060026 (2006).
31 Campan, M., Weisenberger, D. J., Trinh, B. & Laird, P. W. in DNA Methylation 325-337 (2009).
32 Venkatesan, B. M. & Bashir, R. Nanopore sensors for nucleic acid analysis. Nat Nanotechnol 6, 615-624, doi: Doi 10.1038/Nnano.2011.129 (2011).
33 Wanunu, M. et al. Rapid electronic detection of probe-specific microRNAs using thin nanopore sensors. Nat Nanotechnol 5, 807-814, doi: Doi 10.1038/Nnano.2010.202 (2010).
34 Wang, Y., Zheng, D. L., Tan, Q. L., Wang, M. X. & Gu, L. Q. Nanopore-based detection of circulating microRNAs in lung cancer patients. Nat Nanotechnol 6, 668-674, doi: Doi 10.1038/Nnano.2011.147 (2011).
35 Branton, D. et al. The potential and challenges of nanopore sequencing. Nat Biotechnol 26, 1146-1153, doi: Doi 10.1038/Nbt.1495 (2008).
36 Venkatesan, B. M. et al. Highly Sensitive, Mechanically Stable Nanopore Sensors for DNA Analysis. Adv Mater 21, 2771 (2009).
37 Venkatesan, B. M., Shah, A. B., Zuo, J. M. & Bashir, R. DNA Sensing using Nano-crystalline Surface Enhanced ΔI (2)O(3) Nanopore Sensors. Advanced functional materials 20, 1266-1275, doi:10.1002/adfm.200902128 (2010).
38 Shim, J., Rivera, J. A. & Bashir, R. Electron beam induced local crystallization of HfO2 nanopores for biosensing applications. Nanoscale 5, 10887-10893, doi:10.1039/c3nr02608f (2013).
39 Shim, J. et al. Detection and quantification of methylation in DNA using solid-state nanopores. Scientific reports 3, 1389, doi:10.1038/srep01389 (2013).
40 Wanunu, M. et al. Rapid electronic detection of probe-specific microRNAs using thin nanopore sensors. Nat Nanotechnol 5, 807-814, doi:10.1038/nnano.2010.202 (2010).
41 Venta, K. et al. Differentiation of short, single-stranded DNA homopolymers in solid-state nanopores. ACS nano 7, 4629-4636, doi:10.1021/nn4014388 (2013).
42 Ahlquist, D. A. et al. Colorectal cancer screening by detection of altered human DNA in stool: feasibility of a multitarget assay panel. Gastroenterology 119, 1219-1227 (2000).
43 Jorgensen, H. F., Adie, K., Chaubert, P. & Bird, A. P. Engineering a high-affinity methyl-CpG-binding protein. Nucleic Acids Res 34, e96, doi:10.1093/nar/gk1527 (2006).
44 Buck-Koehntop, B. A. et al. Molecular basis for recognition of methylated and specific DNA sequences by the zinc finger protein Kaiso. Proceedings of the National Academy of Sciences of the United States of America 109, 15229-15234, doi:10.1073/pnas. 1213726109 (2012).
45 Buck-Koehntop, B. A., Martinez-Yamout, M. A., Dyson, H. J. & Wright, P. E. Kaiso uses all three zinc fingers and adjacent sequence motifs for high affinity binding to sequence-specific and methyl-CpG DNA targets. FEBS letters 586, 734-739, doi:10.1016/j.febslet.2012.01.045 (2012).
46 Ohki, I. et al. Solution structure of the methyl-CpG binding domain of human MBD1 in complex with methylated DNA. Cell 105, 487-497 (2001).
47 Freedman, M. L. et al. Common variation in BRCA2 and breast cancer risk: a haplotype-based analysis in the Multiethnic Cohort. Human Molecular Genetics 13, 2431-2441 (2004).
48 Haiman, C. A. et al. A comprehensive haplotype analysis of CYP19 and breast cancer risk: the Multiethnic Cohort. Human Molecular Genetics 12, 2679-2692 (2003).
49 Kawano, R., Takeshima, Y. & Inai, K. Effects of K-ras gene mutations in the development of lung lesions induced by 4-(N-methyl-N-nitrosamino)-1-(3-pyridyl)-1-butanone in A/J mice. Jpn. J. Cancer Res. 87, 44-50 (1996).
50 Rodenhuis, S. & Slebos, R. J. C. THE RAS ONCOGENES IN HUMAN LUNG-CANCER. Am. Rev. Respir. Dis. 142, S27-S30 (1990).
51 Siegfried, J. M. et al. Prognostic value of specific KRAS mutations in lung adenocarcinomas. Cancer Epidemiology Biomarkers & Prevention 6, 841-847 (1997).
52 Banerjee, S. et al. Electrochemistry at the edge of a single graphene layer in a nanopore. ACS nano 7, 834-843, doi:10.1021/nn305400n (2013).
53 Lanes, O., Guddal, P. H., Gjellesvik, D. R. & Willassen, N. P. Purification and characterization of a cold-adapted uracil-DNA glycosylase from Atlantic cod (Gadus morhua). Comparative biochemistry and physiology. Part B, Biochemistry & molecular biology 127, 399-410 (2000).
54 Wanunu, M., Morrison, W., Rabin, Y., Grosberg, A. Y. & Meller, A. Electrostatic focusing of unlabelled DNA into nanoscale pores using a salt gradient. Nat Nanotechnol 5, 160-165, doi:10.1038/nnano.2009.379 (2010).

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

When a group of substituents is disclosed herein, it is understood that all individual members of that group and all subgroups, are disclosed separately. When a Markush group or other grouping is used herein, all individual members of the group and all combinations and subcombinations possible of the group are intended to be individually included in the disclosure.

Every formulation or combination of components described or exemplified herein can be used to practice the invention, unless otherwise stated.

Whenever a range is given in the specification, for example, a temperature range, a size range, a parameter range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

TABLE 1
Comparison of Experimental Results of unMethDNA and hyMethDNA/MBD1x
at Various Lengths^{a}

| | | | DNA | Complex | | DNA | Complex | |
|---|---|---|---|---|---|---|---|---|
| | | Nanopore | Current | Current | | Transport | Transport | |
| DNA | No. of | Diameter | Blockage | Blockage | Complex/ | Duration | Duration | Complex/ |
| length | mCpG | (nm) | (nA) | (nA) | DNA | (ms) | (ms) | DNA |
| 90 bp | 10 | 7.70 | 0.54 | 3.50 | 6.52 | 0.12 | 2.86 | 23.33 |
| 60 bp | 6 | 9.50 | 0.31 | 1.73 | 5.54 | 0.06 | 0.29 | 4.45 |
| 30 bp | 3 | 7.10 | 0.71 | 1.51 | 2.14 | 0.14 | 0.74 | 5.14 |

^{a}Values are extracted from transport events recorded at 300 mV.

We claim:

1. A method for characterizing a biomolecular parameter of a polynucleotide, the method comprising the steps of:
concentrating a polynucleotide of interest from a sample comprising a heterogeneous mixture of polynucleotides;
providing the concentrated polynucleotide of interest to a first fluid compartment of a solid-state nanopore, wherein the solid-state nanopore separates the first fluid compartment from a second fluid compartment, and a nanopore fluidically connects the first fluid compartment and the second fluid compartment;
establishing an electric potential across the solid-state nanopore to force the polynucleotide of interest from the first fluid compartment to the second fluid compartment via the nanopore; and
monitoring a passage parameter output during passage of the polynucleotide of interest through the nanopore, wherein the passage parameter output depends on the biomolecular parameter status of the polynucleotide of interest;
thereby characterizing the biomolecular parameter of the polynucleotide of interest.

2. The method of claim 1, wherein the biomolecular parameter is selected from the group consisting of:
an oxidative modification;
an epigenetic modification; and
a nucleotide sequence of interest.

3. The method of claim 1, wherein the biomolecular parameter is methylation.

4. The method of claim 3, wherein the methylation is hypermethylation.

5. The method of claim 3, wherein the methylation is a pattern of methylation sites in the polynucleotide of interest.

6. The method of claim 1, further comprising the step of:
introducing a biomarker to the polynucleotide of interest prior to passage of the polynucleotide of interest through the nanopore, wherein the biomarker specifically binds to a polynucleotide of interest having the biomolecular parameter.

7. The method of claim 6, wherein the biomarker is selected from the group consisting of: a methylation binding protein; a sequence-specific binding motif; an antibody specific to a nucleotide-binding protein; a base excision repair protein; and a nucleotide-binding protein.

8. The method of claim 6, wherein the biomarker comprises at least one of: Uhrf, MBD, Kaiso family, ZBTB4 or ZBTB38, and the biomolecular parameter is methylation of DNA.

9. The method of claim 8, wherein the passage parameter output is a blockade current, a nanopore transit time, or both a blockade current and a nanopore transit time.

10. The method of claim 9, wherein the blockade current for a methylated DNA polynucleotide:MBD complex is at least 2-fold greater than a blockade current for a corresponding unmethylated DNA polynucleotide traversing the nanopore.

11. The method of claim 6, having a biomarker to polynucleotide of interest ratio that is greater than 1:1.

12. The method of claim 1, wherein the polynucleotide is a single stranded DNA, a double stranded DNA or a RNA.

13. The method of claim 1, wherein the polynucleotide has a nucleotide length that is greater than or equal to 30 nucleotides and less than or equal to 100 nucleotides.

14. The method of claim 1, wherein the passage parameter output is selected from the group consisting of: blockade current, threshold voltage, pattern of blockade current, frequency of blockade current, duration of blockade current, translocation velocity, and translocation time.

15. The method of claim 14, further comprising the step of binding a biomarker to the polynucleotide of interest, wherein a binding complex comprising the biomarker and polynucleotide of interest changes an average passage parameter output value by at least 100% compared to a polynucleotide of interest without the bound biomarker.

16. The method of claim 1, wherein the nanopore has an average diameter that is greater than or equal to 5 nm and less than or equal to 12 nm.

17. The method of claim 16, wherein the solid state nanopore comprises a dielectric membrane having a thickness less than or equal to 20 nm.

18. The method of claim 17, wherein the dielectric membrane comprises SiN, Al2O3, graphene, or HfO₂.

19. The method of claim 18, wherein the dielectric membrane comprises graphene having a thickness of less than 0.5 nm through which the nanopore traverses.

20. The method of claim 1, wherein the sample comprises:
a biologic sample obtained from an individual, the biological sample selected from the group consisting of a blood sample, a stool sample, urine sample, a saliva or sputum sample, or a tissue sample.

21. The method of claim 1, wherein the concentrating step comprises:
binding the polynucleotide of interest to a capture element;
separating unbound polynucleotides from the bound polynucleotides of interest; and
releasing the polynucleotide of interest from the capture element.

22. The method of claim 21, wherein the released polynucleotide of interest is transported to the first fluid compartment.

23. The method of claim 21, wherein said capture element is positioned in said first fluid compartment.

24. The method of claim 21, further comprising the step of introducing a biomarker specific to the polynucleotide of interest before binding of the polynucleotide of interest to the capture element or the biomarker is connected to the capture element to capture the polynucleotide of interest.

25. The method of claim 21, further comprising the step of introducing a biomarker specific to the polynucleotide of interest:
after binding of the polynucleotide of interest to the capture element; or
after releasing of the polynucleotide of interest from the capture element.

26. The method of any of claims 21-25, wherein the concentrating step increases a polynucleotide of interest concentration by at least a factor of 500 in a region adjacent to the nanopore compared to the polynucleotide of interest concentration in a region that is not adjacent to the nanopore.

27. The method of claim 26, wherein the first fluid compartment has a sample-containing volume that is fluidically adjacent to a nanopore entrance that is less than or equal to 100 μL.

28. The method of claim 1, further comprising the step of transporting the polynucleotide of interest to the first fluid compartment is by a microfluidic channel.

29. The method of claim 21:
wherein the capture element comprises a magnetic bead to which the polynucleotide of interest is attached, and the capture element is suspended in a microfluidic channel;
wherein the concentrating step further comprises:
applying a magnetic force to drive the magnetic bead with polynucleotide of interest from the microfluidic channel to a first fluid compartment region fluidically adjacent to a nanopore entrance;
introducing a cleavage element into the microfluidic channel and fluidically flowing the cleavage element to the first fluid compartment region to cleave the polynucleotide of interest from the magnetic bead at a cleavable linker site;
wherein the establishing the electric potential step forces polynucleotide of interest in the first fluid compartment region to the nanopore entrance and through the nanopore and the monitoring the passage parameter output distinguishes between biomarker and polynucleotide of interest complexes traversing the nanopore from polynucleotide of interest without biomarker traversing the nanopore.

30. The method of claim 29, wherein the cleavage element during the establishing the electric potential step is positively charged, and the established electric field forces the cleavage element in a direction that is away from the nanopore entrance.

31. The method of claim 29, wherein the cleavable linker site comprises four uracils positioned between an amino conjugation terminal and a complementary sequence

32. The method of claim 31, wherein the cleavage element is a glycosylase that selectively cleaves the cleavable linker site.

33. The method of claim 29, further comprising the step of introducing a biomarker into the microfluidic channel and fluidically flowing the biomarker to the first fluid compartment region to bind the biomarker to polynucleotide of interest having a biomolecular parameter that provides specific binding to the biomarker.

34. The method of claim 24 or 25, wherein the biomarker is a MBD protein.

35. The method of claim 1, wherein the concentrating step comprises providing the polynucleotide of interest to a first fluid compartment region having a confined volume.

36. The method of claim 35, wherein the confined volume is within 500 μm of an entrance of the nanopore or has a confined volume that is less than or equal to 50,000 μm3.

37. The method of claim 35, further comprising the step of directing a magnetic force through a microfluidic channel containing the polynucleotide of interest bound to a magnetic bead flowing through the microfluidic channel to capture magnetic beads within the confined volume.

38. The method of claim 37, wherein the magnetic force is generated by a permanent magnet or a pattern of microfabricated magnets.

39. The method of claim 38, wherein the pattern of microfabricated magnets comprises a ferromagnetic material arranged in a pattern to decrease velocity of the magnetic bead flowing in the microfluidic channel and to increase distribution uniformity of the magnetic beads in a region adjacent to the nanopore entrance.

40. The method of claim 35, further comprising the step of directing a magnetic force through a microfluidic channel containing a magnetic bead flowing through the microfluidic channel to capture magnetic beads within the confined volume, wherein the magnetic beads are coated with an oligonucleotide complementary to a target sequence of the polynucleotide of interest.

41. The method of claim 39, further comprising the step of providing a polynucleotide of interest to the magnetic bead to bind the polynucleotide of interest to the magnetic bead.

42. The method of claim 21, wherein the capture element comprises a particle positioned within a concentrating electric field that directs the particle to the first fluid compartment.

43. The method of claim 42, wherein the particle is a charged bead to which the polynucleotide of interest in attached.

44. The method of claim 42, wherein concentrating electric field is applied in a dielectrophoretic or isotachophoretic manner.

45. The method of claim 1, further comprising the step of selecting a nanopore passage geometry to provide an intermittent interaction between the polynucleotide of interest transiting the nanopore and an inner surface of the nanopore, corresponding to the biomolecular parameter, wherein the intermittent interaction is detectable as a change in passage parameter output.

46. The method of claim 45, wherein biomolecular parameter comprises a nucleotide binding protein that is specific to the biomolecular parameter.

47. The method of claim 46, wherein the biomolecular parameter is methylation and the nucleotide binding protein is a MBD protein.

48. The method of claim 45, wherein at least a portion of the nanopore is functionalized with an antibody for specific binding to the biomolecular parameter during transit of the polynucleotide of interest.

49. The method of claim 1, wherein the polynucleotide of interest comprise a plurality of polynucleotides formed from a first population of polynucleotides having the biomolecular parameter of interest and a second population of polynucleotides without the biomolecular parameter of interest, the method further comprising identifying a fraction of polynucleotides having the bimolecular parameter of interest.

50. The method of claim 1, wherein the polynucleotide of interest is present in the sample at a ratio of less than 1 polynucleotide of interest to 1000 polynucleotides.

51. The method of claim 1, capable of characterizing the biomolecular parameter at a polynucleotide of interest concentration that is as low as 1000 molecules/μL or about 1 fM.

52. The method of any of claims 1-51 for screening a blood sample or a stool sample for a biomolecular parameter indicative of a disease state.

53. The method of claim 52, wherein the disease state is cancer, neurodegeneration, single nucleotide polymorphisms associated with a genetic disease.

54. The method of claim 1, wherein the concentrating step comprises:
providing a bead having a probe connected to a surface of the bead that specifically binds to a polynucleotide of interest.

55. The method of claim 54, wherein the probe comprises a biomarker that specifically binds to a polynucleotide of interest having the biomolecular parameter to be characterized.

56. The method of claim 55, wherein the probe comprises a methyl-binding protein that specifically binds a methylated region of the polynucleotide of interest.

57. The method of claim 56, wherein the methyl binding protein binds to a hemi-methylated region of double-stranded DNA.

58. The method of claim 1, having a sensitivity capable of detecting a single biomolecular parameter in the polynucleotide of interest.

59. The method of claim 58, wherein the biomolecular parameter is cytosine methylation.

60. The method of claim 28, wherein the transporting step comprises decreasing polynucleotide of interest flow velocity in a region adjacent to a nanopore entrance.

61. An integrated diagnostic system comprising:
a solid state nanopore that traverses a dielectric membrane,
the nanopore having a diameter less than 20 nm;
the membrane having a thickness less than 30 nm and a top and a bottom surface with the thickness extending therebetween;
a nanopore entrance coincident with the dielectric membrane top surface;
a first fluid compartment positioned adjacent to the dielectric membrane top surface, and a first fluid compartment region positioned within the first fluid compartment and fluidically adjacent to the nanopore entrance;
a nanopore exit coincident with the dielectric membrane bottom surface, wherein the nanopore fluidically connects the first fluid compartment and the second fluid compartment;
a power supply electrically connected to the first fluid compartment and the second fluid compartment to provide an electric potential difference between the first fluid compartment and the second fluid compartment;
a detector operably connected to the nanopore, the detector configured to monitor a passage parameter output for a polynucleotide traversing the nanopore under the electric potential difference between the first fluid compartment and the second fluid compartment;
a microfluidic passage configured to fluidically transport a sample to the first fluid compartment region;
a capture element positioned in the microfluidic passage and/or the first fluid compartment region for capturing and concentrating a polynucleotide of interest in the first fluid compartment region;
a release element in fluidic contact with the microfluidic passage for controllably releasing the polynucleotide of interest from the capture element to the first fluid compartment region;
wherein upon energization of the power supply, the released polynucleotide of interest in the first fluid compartment region traverses the nanopore to the second fluid compartment.

62. The system of claim 61, further comprising a biomarker in fluidic contact with the microfluidic passage for binding to a polynucleotide of interest having a biomolecular parameter that provides specific binding with the biomarker.

63. The system of claim 61, further comprising a magnet positioned to provide a magnetic force to capture a capture element that is a magnetic particle at the first fluidic compartment region, wherein the first fluidic compartment region is within 500 μm of the nanopore entrance.

64. The system of claim 63, wherein the magnet comprises a plurality of ferromagnetic elements arranged in magnetic contact with the microfluidic channel and in a pattern configured to decrease velocity of a magnetic particle flowing in the microfluidic channel, capture and uniformly distribute magnetic particles relative to the nanopore entrance.

65. The system of claim 64, wherein at least 70% of all magnetic particles flowing in the microfluidic channel are captured by the magnetic force and positioned around the nanopore entrance.

66. The system of claim 61, wherein the microfluidic passage has a cross-sectional area to flow and the first fluid compartment region has a maximum cross-sectional area to flow, wherein the ratio of the first fluid compartment region to microfluidic passage cross-sectional area to flow is greater than or equal to 100.

67. The system of claim 63, wherein the release element comprises an enzyme that selectively cleaves the polynucleotide of interest from the magnetic particle at a cleavable linker site to release polynucleotide of interest to the first fluidic compartment region.
